(19) United States
(12) Patent Application Publication
(10) Pub. No.: US 2025/0195443 A1
(43) Pub. Date: Jun. 19, 2025

(54) Polymer Nanoaggregate Pharmaceutical Composition and Use Thereof

(71) Applicant: ANP Technologies, Inc., Newark, DE (US)

(72) Inventors: Ray Yin, Wilmington, DE (US); Jing Pan, Newark, DE (US); Yubei Zhang, Hockessin, DE (US); Kai Qi, Wilmington, DE (US); Qun Sun, Newark, DE (US); Lin Wang, Hockessin, DE (US); Zhiying Zou, Newark, DE (US)

(73) Assignee: ANP Technologies, Inc., Newark, DE (US)

(21) Appl. No.: 18/847,231

(22) PCT Filed: Mar. 13, 2023
(86) PCT No.: PCT/US23/15121

§ 371 Date: Sep. 14, 2024

Related U.S. Application Data
(60) Provisional application No. 63/319,585, filed on Mar. 14, 2022.
(60) Provisional application No. 63/335,408, filed on Apr. 27, 2022.
(60) Provisional application No. 63/382,149, filed on Nov. 3, 2022.

(51) Int. Cl. A61K 9/51; A61K 9/00; A61K 31/496; A61K 31/5377; B82Y 5/00

(52) U.S. Cl. CPC A61K 9/5146; A61K 9/0019; A61K 31/496; A61K 31/5377; B82Y 5/00

(57) ABSTRACT

This disclosure is directed to a pharmaceutical composition for treating or preventing a disease. The pharmaceutical composition can comprise a polymer-drug nanoaggregate having a polymer and at least one bioactive agent that is water insoluble or poorly water soluble. The polymer is water soluble and comprises at least one first terminal group modified with H or a hydrophobic moiety and a second terminal group modified with a hydrophilic moiety and can be a modified symmetrically or asymmetrically branched polymers. This disclosure is also directed to a method for treating or preventing a disease including one or more immune disorders, infectious diseases and cancers using the pharmaceutical composition disclosed herein. The pharmaceutical composition can be a vaccine or an adjuvant for a vaccine.

### FIELD OF THE INVENTION

The present disclosure relates to a pharmaceutical composition that can be used for treatment of a disease in patients in need thereof. The composition can comprise a nanoaggregate formed from a water-soluble polymer and a water insoluble or poorly water soluble bioactive agent.

### BACKGROUND

Synthetic polymers have been shown to have important applications in pharmaceutical formulations as an effective delivery vehicle or other types of excipients.

Symmetrically branched polymers (SBP), such as, dendritic polymers including Starburst dendrimers (or Dense Star polymers) and Combburst dendrigrafts (or hyper comb-branched polymers), are some of the examples. Those polymers often possess: (a) a well-defined core molecule, (b) at least two concentric dendritic layers (generations) with symmetrical (equal length) branches and branch junctures and (c) exterior surface groups, such as, polyamidoamine (PAMAM)-based branched polymers and dendrimers described in U.S. Pat. Nos. 4,435,548; 4,507,466; 4,568,737; 4,587,329; 5,338,532; 5,527,524; and 5,714,166. Other examples include polyethyleneimine (PEI) dendrimers, such as, those disclosed in U.S. Pat. No. 4,631,337; polypropyleneimine (PPI) dendrimers, such as, those disclosed in U.S. Pat. Nos. 5,530,092; 5,610,268; and 5,698,662; Frechet-type polyether and polyester dendrimers, core shell tectodendrimers and others, as described, for example, in, “Dendritic Molecules,” edited by Newkome et al., VCH Weinheim, 1996, “Dendrimers and Other Dendritic Polymers,” edited by Frechet & Toroalia, John Wiley & Sons, Ltd., 2001; and U.S. Pat. No. 7,754,500.

Combburst dendrigrafts are constructed with a core molecule and concentric layers with symmetrical branches through a stepwise synthetic method. In contrast to dendrimers, Combburst dendrigrafts or polymers are generated with monodisperse linear polymeric building blocks (U.S. Pat. Nos. 5,773,527; 5,631,329 and 5,919,442). Moreover, the branch pattern is different from that of dendrimers. For example, Combburst dendrigrafts form branch junctures along the polymeric backbones (chain branches), while Starburst dendrimers often branch at the termini (terminal branches). Due to the living polymerization techniques used, the molecular weight distributions (M_{w}/Mₙ) of those polymers (core and branches) often are narrow. Thus, Combburst dendrigrafts produced through a graft-on-graft process are well defined with M_{w}/Mₙ ratios often approaching 1.

SBPs, such as, dendrimers, are produced predominantly by repetitive protecting and deprotecting procedures through either a divergent or a convergent synthetic approach. Since dendrimers utilize small molecules as building blocks for the cores and the branches, the molecular weight distribution of the dendrimers often is defined. In the case of lower generations, a single molecular weight dendrimer often is obtained. While dendrimers often utilize small molecule monomers as building blocks, dendrigrafts use linear polymers as building blocks.

In addition to dendrimers and dendrigrafts, other SBPs can include symmetrical star-shaped or comb-shaped polymers, such as, symmetrical star-shaped or comb-shaped polyethyleneoxide (PEO), polyethyleneglycol (PEG), polyethyleneimine (PEI), polypropyleneimine (PPI), polyoxazoline (POX), polymethyloxazoline (PMOX), polyethyloxazoline (PEOX), polypropyloxazoline (PPOX), polystyrene, polymethylmethacrylate (PMMA), or polydimethylsiloxane.

Asymmetrically branched polymers (ABP) can have two different types: regular ABP and random ABP. Asymmetrically branched dendrimers or regular ABPs (reg-ABPs), often possess a core, controlled and well-defined asymmetrical (unequal length) branches and asymmetrical branch junctures as described in U.S. Pat. Nos. 4,289,872; 4,360,646; and 4,410,688. On the other hand, a random ABP (ran-ABP) possesses: a) no core, b) functional groups both at the exterior and in the interior, c) random/variable branch lengths and patterns (i.e., termini and chain branches), and d) unevenly distributed interior void spaces.

The synthesis and mechanisms of ran-ABPs, such as, those made from PEI, were reported by Jones et al., J. Org. Chem. 9, 125 (1944), Jones et al., J. Org. Chem. 30, 1994 (1965) and Dick et al., J. Macromol. Sci. Chem., A4 (6), 1301-1314, (1970)). Ran-ABP, such as, those made of POX, poly(2-oxazoline), poly(2-methyloxazoline) (PMOX) and poly(2-ethyloxazoline) (PEOX), was reported by Litt (J. Macromol. Sci. Chem. A9(5), 703-727 (1975)) and Warakomski (J. Polym. Sci. Polym. Chem. 28, 3551 (1990)). The synthesis of ran-ABP's often can involve a one-pot divergent or a one-pot convergent method.

A polymer can also be a homopolymer or a copolymer. A copolymer is a polymer, or a polymer backbone, polymerized from different monomers or different monomer repeating units. A homopolymer can relate to a polymer or a polymer backbone composed of the same repeat unit, that is, the homopolymer is generated from the same monomer. The monomer can be a simple compound or a complex or an assemblage of compounds where the assemblage or complex is the repeat unit in the homopolymer.

Although branched polymers, including SBPs and ABPs, have been used for drug delivery, those attempts are focused primarily on the chemical attachment of the drug to the polymer, or physical encapsulation of such drugs in the interior through unimolecular encapsulation (such as, those described in U.S. Pat. Nos. 5,773,527; 5,631,329; 5,919,442; and 6,716,450). For example, dendrimers and dendrigrafts are believed to physically entrap bioactive molecules using unimolecular encapsulation approaches, as described in U.S. Pat. Nos. 5,338,532; 5,527,524; and 5,714,166 for dense star polymers, and U.S. Pat. No. 5,919,442 for hyper comb-branched polymers. Similarly, the unimolecular encapsulation of various drugs using SBPs to form a “dendrimer box” was reported in Tomalia et al., Angew. Chem. Int. Ed. Engl., 1990, 29, 138, and in “Dendrimers and Other Dendritic Polymers”, edited by Frechet & Tomalia, John Wiley & Sons, Ltd., 2001, pp. 387-424.

Branched core shell polymers with a hydrophobic core and a hydrophilic shell may be used to entrap a poorly water soluble drug through molecular encapsulation. Randomly branched and hyperbranched core shell structures with a hydrophilic core and a hydrophobic shell have also been used to carry a drug through unimolecular encapsulation and pre-formed nanomicelles (U.S. Pat. No. 6,716,450 and Liu et al., Biomaterials 2010, 10, 1334-1341). However, those unimolecular and pre-formed micelle structures are generated in the absence of a drug.

Block copolymers, such as, miktoarm polymers (i.e., Y shaped/AB₂-type star polymers) and linear (A)-dendritic (B) block copolymers, were observed to form stereocomplexes with paclitaxel (Nederberg et al., Biomacromolecules 2009, 10, 1460-1468 and Luo et al., Bioconjugate Chem. 2010, 21, 1216). Those block copolymers closely resemble traditional lipid or AB-type linear block copolymers, which are well known surfactants used for the generation of micelles. However, such branched block copolymers are difficult to make and thus, are not suitable for mass production.

Water insoluble or poorly water soluble bioactive agents are difficult to formulate. Typically, multiple surfactants, detergents and other materials or a complex high energy emulsification process can be needed. Large biological molecules, such as, albumin, have been used in certain formulations for water insoluble paclitaxel, such as, Abraxane® available from Celgene and Bristol-Myers Squibb under respective trademark. However, availability and large-scale production of such biological molecules have presented significant challenges.

Vaccines can help the body recognize and destroy certain targets, such as, cancer cells or microorganisms that cause infections. Adjuvants are typically used to modify, augment, or increase the efficacy or potency of a vaccine to provide better immunity to a particular disease. Aluminum-containing adjuvants have been used in vaccines since 1930s. Small amounts of aluminum are added to help the body build stronger immunity against microorganisms. Monophosphoryl lipid A (MPL) (also known as “AS04”) was used in U.S. vaccine (Cervarix®) and can have immune-boosting effects. An oil-in-water emulsion-based adjuvant MF59 contains squalene, a naturally occurring oil found in many plant and animal cells, as well as in humans. The MF59 adjuvant has been used in Fluad (an influenza vaccine licensed for adults aged 65 or older) in Europe since 1997 and in the United States since 2016. Another adjuvant, AS01B, is an adjuvant suspension used with the antigen component of Shingrix vaccine. AS01B is made of monophosphoryl lipid A (MPL) and QS-21, a natural compound extracted from the Chilean soapbark tree (Quillaja saponaria Molina). AS01B is also a component of vaccines currently being tested in clinical trials, including malaria and HIV vaccines. CpG 1018, a 22-mer CpG ODN containing sequence with a modified phosphorothioate backbone, is a recently developed adjuvant used in Heplisav-B® vaccine (registered trademark of Dynavax Technologies Corporation). It contains synthetic oligodeoxynucleotides with cytosine phosphoguanine (CpG) motifs (CpG ODN) that are agonists for TLR9 and mimic the activity of naturally occurring CpG motifs found in DNA foreign to the body, such as, bacterial and viral DNAs.

There is a continued need for new pharmaceutical formulations that can deliver drugs more effectively or to improve vaccine efficacy by stimulating better immunity.

### SUMMARY

In some cases, present invention is directed to a pharmaceutical composition comprising: a nanoaggregate comprising a polymer and at least one bioactive agent that is water insoluble or poorly water soluble; and optionally a pharmaceutical suitable carrier; wherein the pharmaceutical composition is soluble in an aqueous solution to produce at least 1 mg/mL of the bioactive agent in the aqueous solution; wherein the polymer is water soluble; and wherein the polymer comprises: a first polymer comprising at least one first terminal group modified with H or a hydrophobic moiety and a second terminal group modified with a hydrophilic moiety, wherein the first terminal group comprises in a range of from 1% to 99% of H and 1% to 99% of the hydrophobic moiety that comprises saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

In some cases, the pharmaceutical composition can be a drug for treating or preventing a disease selected from one or more immune disorders, infectious diseases, cancers, and a combination thereof.

In some cases, the pharmaceutical composition can be an adjuvant for a vaccine.

In some cases, the pharmaceutical composition can be a prophylactic vaccine, a therapeutic vaccine, or a combination thereof, wherein the pharmaceutical composition further comprises at least one immune agent for stimulating immune response in a subject in need thereof.

In some cases, present invention is directed to a method for treating or preventing a disease of a subject in need thereof, the method comprising administering the subject with an effective dose of a pharmaceutical composition disclosed herein.

In some cases, present invention is directed to a nanoaggregate comprising a polymer and at least one bioactive agent that is water insoluble or poorly water soluble; wherein the nanoaggregate is soluble in an aqueous solution to produce at least 1 mg/mL of the bioactive agent in the aqueous solution; wherein the polymer is water soluble; wherein the bioactive agent comprises a natural or synthetic small molecule-based drug, inorganic-based drug, biological drug, natural or synthetic large molecule-based drug, a derivative thereof, or a combination thereof; and wherein the polymer comprises: a first polymer comprising at least one first terminal group modified with H or a hydrophobic moiety and a second terminal group modified with a hydrophilic moiety, wherein the first terminal group comprises in a range of from 1% to 99% of H and 1% to 99% of the hydrophobic moiety that comprises saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

### BRIEF DESCRIPTION OF THE FIGURES

FIG. 1A-FIG. 1D. Examples of SBPs including (FIG. 1A) a dendrimer, (FIG. 1B) a dendrigraft, (FIG. 1C) a regular comb-branched polymer and (FIG. 1D) a star-branched polymer. All have a core, either globular or linear.

FIG. 2A and FIG. 2B. Examples of chemical structures of symmetrically branched polypropyleneimine (PPI) dendrimers. FIG. 2A: A dendrimer with 4-PPI. FIG. 2B: A dendrimer with additional 8-PPI.

FIG. 3. Examples of chemical modification reactions of symmetrically branched PPI dendrimers. The numbers, 8, 16, 32, 64, 128 and so on, indicate the number of reactive groups at the surface of the dendrimer.

FIG. 4A and FIG. 4B. Schematic examples of random (FIG. 4A) and regular (FIG. 4B) asymmetrically branched polymers (ABPs) with asymmetric branch junctures and patterns.

FIG. 5. An example of a chemical structure of a random asymmetrically branched PEI homopolymer.

FIG. 6A-FIG. 6C. Examples of synthetic schemes. FIG. 6A: Chemical modification reactions of random asymmetrically branched PEI homopolymers.

FIG. 6B: Example of a one-pot synthesis of hydrophobically modified, randomly branched poly(2-ethyloxazoline) with a primary amino group at the focal point of the polymer. The initiator/surface group (1) is a brominated hydrocarbon. The reaction opens the oxazoline ring. FIG. 6C: Unlimited examples of polymers having different first terminal and second terminal groups.

FIG. 7A and FIG. 7B. Schematic examples of illustrations of a drug loaded in or at the surface domain or region of the branched polymer (FIG. 7A) SBP's and (FIG. 7B) ABP's. In this and other figures, R indicates a surface group and a solid circle depicts a bioactive agent, such as, a drug of interest.

FIG. 8. A schematic illustration of an example of nanoparticles containing both drug molecules (solid circle) and branched polymers with surface groups (R).

FIG. 9A and FIG. 9B. Schematic examples of illustrations of a water insoluble or poorly water soluble drug that is loaded at hydrophobic surface groups of branched polymers (FIG. 9A) SBP's and/or (FIG. 9B) ABP's. In this and other figures, a thin wavy line depicts a hydrophobic surface group.

FIG. 10A and FIG. 10B. Schematic examples of various drug-containing nanoparticles (FIG. 10A) SBP and (FIG. 10B) ABP also carrying at least one targeting group or moiety, such as, an antibody, depicted herein and in other figures as a “Y”.

FIG. 11A-FIG. 11C. Examples of Light scattering (LS) measurement data of nanoaggregates. FIG. 11A: Polymer A1 and Rapamycin at 5:1 ratio.

FIG. 11B: Polymer A1 and Rapamycin at 7.5:1 ratio. FIG. 11C: Polymer B1 and Rapamycin at 5:1 ratio.

FIG. 12A-FIG. 12D. Formula of examples of STING agonists. FIG. 12A: Formula (1)-(6). FIG. 12B: Formula (7)-(12). FIG. 12C: Formula (13)-(18). FIG. 12D: Formula (19)-(24). FIG. 12E: Formula (25)-(29).

FIG. 13A-FIG. 13E. Representative molecules of examples of bioactive agents. FIG. 13A: SN-38, 7-ethyl-10-hydroxycamptothecin. FIG. 13B: irinotecan, also known as CPT-11, Camptosar®, Campto, Onivyde® under respective trademarks. FIG. 13C: Camptothecin (CPT). FIG. 13D: Topotecan, also known as Hycamtin. FIG. 13E: an example of SN-38 ADC, TRODELVY® (hRS7-SN38 ADC), under respective trademark.

FIG. 14A-FIG. 14E. Representative measurement data on particles of nanoaggregates. FIG. 14A: one example of nanoaggregates (Formulation 1). FIG. 14B: another example of nanoaggregates (Formulation 2). FIG. 14C: another example of nanoaggregates (Formulation 3). FIG. 14C: another example of nanoaggregates (Formulation 3). FIG. 14D: another example of nanoaggregates (Formulation 4). FIG. 14E: another example of nanoaggregates (Formulation 5).

FIG. 15. Representative SN-38 cytotoxicity data using the HCT-116 Cell line.

### DETAILED DESCRIPTION

Features and advantages of the present invention will be more readily understood, by those of ordinary skill in the art, from reading the following detailed description. It is to be appreciated that certain features of the invention, which are described above and below in the context of separate embodiments, may also be provided in combination in a single embodiment. Conversely, various features of the invention that are, for brevity, described in the context of a single embodiment, may also be provided separately or in any combination or sub-combination. In addition, references in the singular may also include the plural (for example, “a” and “an” may refer to one, or one or more) unless the context specifically states otherwise.

Use of numerical values in the various ranges specified in this application, unless expressly indicated otherwise, are stated as approximations as though minimum and maximum values within the stated ranges were both proceeded by the word, “about”. In this manner, slight variations above and below the stated ranges can be used to achieve substantially the same results as values within the ranges. Also, disclosure of ranges is intended as a continuous range including every value between the minimum and maximum values and including the minimum and maximum cited values.

The drug solubility in the instant disclosure is defined as, relative to parts of solvent required to solubilize one part of bioactive agent or drug, <30 (soluble), 30-100 (poorly soluble) and >100 (insoluble). Water solubility is defined herein as, relative to parts of water required to solubilize one part of bioactive agent or drug, <30 (water soluble), 30-100 (poorly water soluble) and >100 (water insoluble).

For the purposes of the instant disclosure, a randomly branched PEI, although there are branches of different length and branches occur randomly, is considered as a homopolymer because that branched polymer is composed of a single monomer, the ethyleneimine or aziridine repeat unit. A polymer having a structure of “(AB)-(AB)-(AB)- . . . ” can also be considered as a homopolymer because of the (AB) repeating unit. The homopolymer may be linear or branched. Also, one or more of the monomer or complex monomer components can be modified, substituted, derivatized and so on, for example, modified to carry a functional group. Such molecules are homopolymers for the purposes of the instant disclosure as the polymer backbone is composed of a single type of simple or complex monomer.

The term “polymer” refers to any polymer suitable for this invention as defined above and hereafter. In examples, a polymer can comprise polyoxazoline or modified polyoxazoline as disclosed herein. In further examples, the polymer can comprise a modified polyoxazoline can comprise one or more second terminal groups, such as, an —NH₂, —NH, —NH₃⁺, other basic groups or a combination thereof, with a proviso that in a range of from 0.01% to 100% of the second terminal group is free from primary amine. In some cases, in a range of from 0.01% to 100%, 0.1% to 100%, or 1% to 100%, of the second terminal group is free from primary amine. In some cases, in a range of from 1% to 100% of the second terminal group can comprise hydroxyl group. All percentages are based on the total number of the second terminal groups.

The term “bioactive agent” or “bioactive agents” refers to a molecule, a compound, a complex of one or more compounds or molecules, or a combination thereof that can provide a biological activity in vivo, in vitro, or a combination thereof. A pharmaceutical composition can comprise one or more bioactive agent, such as, pharmaceutically active agents (PAAs) or active pharmaceutical ingredients (APIs), and other bioactive or inert compounds that can include emollients, bleaching agents, antiperspirants, pharmaceuticals, moisturizers, scents, colorants, pigments, dyes, antioxidants, oils, fatty acids, lipids, inorganic salts, organic molecules, opacifiers, vitamins, pharmaceuticals, keratolytic agents, UV blocking agents, tanning accelerators, depigmenting agents, deodorants, perfumes, insect repellants, or a combination thereof. Some examples of bioactive agents are described in detail in this disclosure.

The term “taxane” refers to paclitaxel, docetaxel, cabazitaxel, larotaxel, milataxel, ortataxel, tesetaxel, or a combination thereof. In some cases, paclitaxel can be preferred.

The term “rapamycin” is also referred to as “Sirolimus” throughout this discloser and may be used interchangeably.

The term “mTOR” refers to mammalian target of rapamycin that includes protein kinase(s) regulating cell growth, survival, metabolism, and immunity. mTOR can be assembled into several complexes, such as, mTOR complex 1 (mTORC1), mTOR complex 2 (mTORC2), and mTOR complex 3 (mTORC3). Activation of mTOR can promote tumor growth and metastasis. Inhibition of mTOR with one or more mTOR inhibitors can be used to treat cancer.

The term “mTOR inhibitor”, “mTOR inhibitors”, “inhibitor of mTOR”, or “inhibitors of mTOR” refers to molecules that inhibits the activity of mTOR or mTOR complexes. Some examples of mTOR inhibitor drugs or molecules can include everolimus, available as AFINITOR® and Tablets/AFINITOR DISPERZ® (trademark of Novartis), Zortress® (Pfizer); temsirolimus, available as Torisel® (Pfizer); sirolimus (rapamycin) available as Rapamune® (Pfizer) and FYARRO™ (trademark of Aadi Bioscience, Inc.); zotarolimus; torin-1; torin-2; vistusertib; ridaforolimus (also known as AP23573 and MK-8669, or deforolimus); one or more dual PI3K-mTOR inhibitors (such as, PKI-402, SPR965, PI-103, GNE477, WJD008, GSK2126458 and others); one or more ATP-competitive mTORC1/2 inhibitors (such as, AZD-8055, OSI-027, INK128, WYE-132, Torin1, and others); Apitolisib (GDC-0980 or RG7422), AZD8055, BGT226, CC-223, CH5132799, Chrysophanic Acid (Chrysophanol), Dactolisib, ETP-46464, GDC-0349, Gedatolisib, GNE-493, GSK1059615, INK 128, KU-0063794, LY3023414, MHY1485, mTOR-IN-1, Omipalisib, OSI-027, Palomid 529, PF-04691502, PF-04979064, PI-103, PP121, QL-IX-55, SF1126, Tacrolimus, Torkinib, Vistusertib, Voxtalisib, Voxtalisib, VS-5584, WAY-600, WYE-125132, WYE-354, WYE-687, XL388, a derivative thereof, or a combination thereof. Commercially available mTOR inhibitors, such as, those available from Adooq Biosciences can be suitable.

SN-38 (7-ethyl-10-hydroxycamptothecin) is a Topoisomerase 1 (herein “Top 1” or “Top I”) inhibitor and a camptothecin derivative. Irinotecan (CPT-11) is a water soluble camptothecin analog and a prodrug of SN-38. Since the discovery of camptothecin in the tree bark of Camptotheca acuminata, the development of camptothecin derivatives as anticancer agents has resulted in the approval of a few drugs, such as, irinotecan for the treatment of colon or colorectal cancer, topotecan for the treatment of small cell lung cancer, ovarian, and cervical cancer, as well as antibody drug conjugates (ADC) using SN-38 as payload. Due to its low solubility and high toxicity, currently there is no approved SN-38 drug product for chemotherapy. Studies on polymer conjugated SN-38 made by linking SN-38 with a multiarm polyethylene glycol via a glycine linker (Sapra, P., et al., Clin. Cancer Res., 14(6):1888, 2008) and polymer micelle SN-38 (Carie, A; et al., J. Drug Delivery, Vol. 2011, p. 9, 2011, doi:10.1155/2011/869027) have been reported. However, synthesis and production of those are complex.

In some cases, other Topoisomerase inhibitors, such as, Topoisomerase II (herein “Top 2” or “Top II”) inhibitors, for example, doxorubicin, etoposide, quinolones, fluoroquinolone, or a combination thereof, can also be suitable for the pharmaceutical composition, process and method disclosed herein. In some cases, quinolones that target two essential bacterial Top 2 enzymes, DNA gyrase and DNA topoisomerase IV, can be suitable. In some cases, the pharmaceutical composition can be suitable for the treatment of infectious diseases.

The term “pharmaceutical suitable carrier”, “pharmaceutical suitable carriers”, “pharmaceutically suitable carrier” or “pharmaceutically suitable carriers” refers to one or more inactive ingredients that are in approved drug products. Inactive ingredients listed in the database “Inactive Ingredients in Approved Drug Products” maintained and updated by US Food and Drug Administration (FDA) can be suitable. In some cases, a pharmaceutical suitable carrier can also be referred to as an excipient.

The term “subject” or “subjects” used throughout this disclosure refers to an animal, a human or a human patient. The term “animal” refers to wild animals, captured or zoo-raised animals and domesticated animals including live stocks, farm animals, pets, laboratory animals, such as, horse, cattle, pig, donkey, mule, camel, goat, sheep, monkey, rabbit, dog, cat, mouse, rat, and the like. Warm-blooded animals are suitable. The term “human” refers to a human patient having one or more diseases in need of a treatment, a person having one or more medical conditions unrelated to a treatment, or a healthy person. In some cases, a subject can be a human patient or a healthy person.

The term “antibody”, “antibodies” or “fragment of an antibody” can include natural or synthetic antibodies that selectively bind to an antigen. The term includes polyclonal and monoclonal antibodies produced from animals, cells including eukaryotic or prokaryotic cells, cell free systems, or chemical synthesis. In addition to intact immunoglobulin molecules, also included in the term “antibodies” are fragments or polymers of those immunoglobulin molecules, and human or humanized versions of immunoglobulin molecules that selectively bind a target antigen.

The term “aqueous solution” or “aqueous solutions” used throughout this disclosure refers to a solution comprises in a range of from 80% to 100% water, percentage based on the total non-solid weight of the aqueous solution. An aqueous solution can further comprise additional components, such as, salt, acid, base, buffer, solvent, organic solvent, particles, emulsion, solids or non-solids, detergents, small molecules, large molecules, other ingredients, or a combination thereof. The term “non-solid weight” refers to the weight from solid contents after the aqueous solution is dried out, such as, by removing all the water or other liquids.

The term “infectious disease” or “infectious diseases” refers to illnesses caused by harmful organisms (pathogens), such as, bacteria, viruses, fungi, protozoa, worms, parasites, prions, a part thereof, or a combination thereof. The infectious diseases can be transmitted among people, from contacting with animals, insects, or from contaminated food, water or soil. Some examples of infectious diseases can include Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, and other known diseases, or diseases that yet to emerge or be identified.

Ther term “vaccine” or “vaccines” refers to a substance or group of substances that are designed to cause the immune system of a subject, such as, humans or animals, to respond to microorganisms, such as, bacteria, viruses, fungi, protozoa, worms, parasites, prions, other harmful organisms (pathogens), or a tumor. A vaccine can help the body recognize and destroy microorganisms or cancer cells. In some cases, a vaccine can comprise a protein, nucleic acids encoding the protein, a toxin, nucleic acids, oligo nucleic acids; DNAs; RNAs; mRNAs; siRNAs; sgRNAs; or a combination thereof, from the microorganisms or from cancer cells. In some cases, a vaccine can comprise a modified protein, nucleic acids encoding the modified protein, a toxin, nucleic acids, modified nucleic acids, oligo nucleic acids, or modified oligo nucleic acids; DNAs; RNAs; mRNAs; siRNAs; sgRNAs; or a combination thereof, that are designed to cause the immune system to respond to the microorganisms or the cancer cells. Modified or synthetic DNAs, RNAs, mRNAs, siRNAs, sgRNAs, or a combination thereof, can also be suitable.

The term “adjuvant” or “adjuvants” refers to a drug, substance, a reagent, or a combination thereof, that is used to modify, augment, or increase the efficacy or potency of a vaccine to provide better immunity to a particular disease. Adjuvants can comprise one or more organic molecules; antigenic molecules that can mimic specific pathogen-associated molecular patterns, which include liposomes, lipopolysaccharide, molecular cages for antigens, components of bacterial cell walls, and endocytosed nucleic acids, such as, RNA, double-stranded RNA (dsRNA), DNA, single-stranded DNA (ssDNA), methylated or unmethylated CpG dinucleotide-containing DNA; inorganic compounds, such as, potassium alum, aluminum hydroxide, aluminum phosphate, calcium phosphate hydroxide; oils, such as, paraffin oil propolis, peanut oil; bacterial products, such as, killed bacteria; plant products, such as, those from soybean or other plants; Cytokines, such as, IL-1, IL-2 or IL-12; or a combination thereof.

The term “isomer” or “isomers” refers to molecules that share the same chemical formula but have their atoms connected differently or arranged differently in space, including structural isomers having respective atoms bonded together in different orders, geometric isomers having atoms bonded in the same order, but differ in the configuration around the bonds, such as, cis- or trans-isomers and enantiomers having the same chemical structure but differ in three-dimensional arrangements of atoms around asymmetric carbon, such that they are mirror images of one another.

In some cases, this disclosure is directed to a pharmaceutical composition comprising:
a nanoaggregate comprising a polymer and at least one bioactive agent that is water insoluble or poorly water soluble; and
optionally a pharmaceutical suitable carrier;
wherein the pharmaceutical composition is soluble in an aqueous solution to produce at least 1 mg/mL of the bioactive agent in the aqueous solution;
wherein the polymer is water soluble; and
wherein the polymer can comprise:
a first polymer comprising at least one first terminal group modified with H or a hydrophobic moiety and a second terminal group modified with a hydrophilic moiety, and wherein the first terminal group comprises in a range of from 1% to 99% of H and 1% to 99% of the hydrophobic moiety that can comprise saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or
a second polymer comprising one or more hydroxyl dendrimers (HD) (also known as hydroxyl terminated dendrimer); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP);
or a combination thereof.

In some cases, the polymer can comprise the first polymer, as disclosed herein comprising at least one first terminal group modified with H or a hydrophobic moiety and a second terminal group modified with a hydrophilic moiety, and wherein the first terminal group comprises in a range of from 1% to 99% of H and 1% to 99% of the hydrophobic moiety that can comprise saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof.

In some cases, the polymer can consist of the first polymer, as disclosed herein comprising at least one first terminal group modified with H or a hydrophobic moiety and a second terminal group modified with a hydrophilic moiety, and wherein the first terminal group comprises in a range of from 1% to 99% of H and 1% to 99% of the hydrophobic moiety that can comprise saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof. In some cases, the pharmaceutical composition can be free from the polymers selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

In some cases, the polymer can comprise a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

In some cases, the polymer can consist of one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof. In some cases, the polymer can comprise only the polymers listed above and be free from the aforementioned polymers comprising the at least one first terminal group modified with H or a hydrophobic moiety and the second terminal group modified with a hydrophilic moiety.

In some cases, the polymer can comprise the first polymer and one or more subsequent polymers (also referred to as “second polymer”) selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

In some cases, the polymer can comprise a polyoxazoline (POX) that comprises a linear portion, a branched portion, or a combination thereof, and wherein the polyoxazoline (POX) can comprises poly(2-methyloxazoline), poly(2-ethyloxazoline), poly(2-propyloxazoline), poly(isopropyloxazoline), or a combination thereof. In some cases, the polyoxazoline can be poly(2-ethyloxazoline).

In some cases, the polyoxazoline can comprise a molar ratio of monomer to initiator in a range of from 50:1 to 80:1.

In some cases, in a range of from 1% to 100% of the second terminal group is free from primary amine. In some cases, the pharmaceutical composition disclosed herein, in a range of from 1% to 100% of the second terminal group can comprise hydroxyl group. All percentages are based on the total number of the second terminal groups.

In some cases, the first terminal group comprises in a range of from 1% to 99% of H and 1% to 99% of the hydrophobic moiety, 1% to 90% of H and 10% to 99% of the hydrophobic moiety, 1% to 85% of H and 15% to 99% of the hydrophobic moiety, 1% to 80% of H and 20% to 99% of the hydrophobic moiety, 1% to 75% of H and 25% to 99% of the hydrophobic moiety, 1% to 70% of H and 30% to 99% of the hydrophobic moiety, 1% to 65% of H and 35% to 99% of the hydrophobic moiety, 1% to 60% of H and 40% to 99% of the hydrophobic moiety, 1% to 55% of H and 45% to 99% of the hydrophobic moiety, 1% to 50% of H and 65% to 99% of the hydrophobic moiety, 1% to 45% of H and 55% to 99% of the hydrophobic moiety, 1% to 40% of H and 60% to 99% of the hydrophobic moiety, 1% to 35% of H and 65% to 99% of the hydrophobic moiety, 1% to 30% of H and 70% to 99% of the hydrophobic moiety, 1% to 25% of H and 75% to 99% of the hydrophobic moiety, 1% to 20% of H and 80% to 99% of the hydrophobic moiety, 1% to 15% of H and 85% to 99% of the hydrophobic moiety, 1% to 10% of H and 90% to 99% of the hydrophobic moiety, 1% to 5% of H and 95% to 99% of the hydrophobic moiety, 1% of H and 99% of the hydrophobic moiety, including all percentages within the range, percentage based on the total number of the first terminal group in the polymer. In some cases, the first terminal group comprises in a range of from 1% to 50% of H and 50% to 99% of the hydrophobic moiety, 1% to 40% of H and 60% to 99% of the hydrophobic moiety, 1% to 30% of H and 70% to 99% of the hydrophobic moiety, 1% to 20% of H and 80% to 99% of the hydrophobic moiety, 1% to 10% of H and 90% to 99% of the hydrophobic moiety, 1% to 5% of H and 95% to 99% of the hydrophobic moiety, or 1% to 2% of H and 98% to 99% of the hydrophobic moiety, including all percentages within the range, percentage based on the total number of the first terminal groups in the polymer. In some cases, the percentage is based on molar numbers of the first terminal groups in the polymer.

Alternatively, a H to hydrocarbon group (“hydrocarbon”) ratio can be used to describe the polymer, such as, H:hydrocarbon=0.01:1 to 100:1. In some cases, the first terminal group comprises a ratio of H:hydrophobic moiety in a range of from 0.01:1 to 100:1, including all ratios within the range. In some cases, the first terminal group comprises a ratio of H:hydrophobic moiety in a range of from 0.01:1 to 100:1, 0.1:1 to 100:1, 0.2:1 to 100:1, 0.5:1 to 100:1, 0.7:1 to 100:1, 1:1 to 100:1, 2.0:1 to 100:1, 5;1 to 100:1, 10:1 to 100:1, 20:1 to 100:1, 30:1 to 100:1, 40:1 to 100:1, 50:1 to 100:1, 60:1 to 100:1, 70:1 to 100:1, 80:1 to 100:1, 90:1 to 100:1, and 95:1 to 100:1, including all ratios within the range. In some cases, the first terminal group comprises a ratio of H:hydrophobic moiety in a range of from 0.01:1 to 10:1, 0.1:1 to 10:1, 0.1:1 to 10:1, 0.2:1 to 10:1, 0.5:1 to 10:1, 0.7:1 to 10:1, 1:1 to 10:1, 2.0:1 to 10:1, 5;1 to 10:1, 10:1, 20:1 to 10:1, 30:1 to 10:1, 40:1 to 10:1, 50:1 to 10:1, 60:1 to 10:1, 70:1 to 10:1, 80:1 to 10:1, 90:1 to 10:1, and 95:1 to 10:1, including all ratios within the range. In some cases, the first terminal group comprises a ratio of H:hydrophobic moiety in a range of from 0.01:1 to 5:1, 0.1:1 to 5:1, 0.1:1 to 5:1, 0.2:1 to 5:1, 0.5:1 to 5:1, 0.7:1 to 5:1, 1:1 to 5:1, 2.0:1 to 5:1, 5;1, 10:1, 20:1 to 5:1, 30:1 to 5:1, 40:1 to 5:1, 50:1 to 5:1, 60:1 to 5:1, 70:1 to 5:1, 80:1 to 5:1, 90:1 to 5:1, and 95:1 to 5:1, including all ratios within the range. In some cases, the first terminal group comprises a ratio of H:hydrophobic moiety can be selected from 0.01:1, 0.1:1, 0.2:1, 0.5:1, 0.7:1, 1:1, 2.0:1, 3.0:1, 4.0:1, 5;1, 6;1, 7:1, 8:1, 9:1, 10:1, 20:1, 30:1, 40:1, 50:1, 60:1, 70:1, 80:1, 90:1, 95:1, and 100:1, including all ratios within the range. The ratio can be based on molar ratio of the H and the hydrocarbon group.

The percentage and ratio can be easily converted by conventional method, for example, a ratio of 0.01:1 can be converted to about 1%, 0.2:1 can be converted to about 17%, 0.5:1 can be converted to about 33%, 1:1 can be converted to about 50%, 1.5:1 can be converted to about 60%, 2:1 can be converted to about 67%, 5:1 can be converted to about 83%, 10:1 can be converted to about 90%, 20:1 can be converted to about 95%, and 100:1 can be converted to about 99%.

The percentage or the ratio of the hydrogen modified first terminal group and the hydrocarbon modified first terminal group can be measured with HPLC as known to those skilled in the art.

In the pharmaceutical composition disclosed herein, the first terminal group can comprise H or a hydrophobic moiety that can comprise saturated or unsaturated aliphatic hydrocarbon having 1 to about 22 carbons, an aromatic hydrocarbon, or a combination thereof, and the second terminal group can comprise a group modified by an amine, amide, imine, imide, carboxyl, hydroxyl, ester, ether, acetate, phosphate, ketone, aldehyde, sulfonate, or a combination thereof.

The first terminal group can comprise hydrogen (H) in one example, a hydrocarbon having 2 to 22 carbons in one example, 4 to 22 carbons in another example, 6 to 22 carbons in yet another example, 7 to 22 carbons in yet another example, 8 to 22 carbons in yet another example, 10 to 22 carbons in yet another example, 12 to 22 carbons in yet another example, 14 to 22 carbons in yet another example, 16 to 22 carbons in yet another example, and 18 to 22 carbons in a further example In one particular example, the first terminal group can comprise 18 carbons, such as, a (CH₃(CH₂)₁₇)— group. In some cases, the first terminal group can comprise a hydrocarbon having 7 to 22 carbons. In some cases, the first terminal group can comprise H. In some cases, the first terminal group can comprise in a range of from 1% to 99% of H and 1% to 99% of the hydrophobic moiety that can comprise saturated or unsaturated aliphatic hydrocarbon having 1 to about 22 carbons, an aromatic hydrocarbon, or a combination thereof. The first terminal group can be modified by selecting various initiators. In some cases, p-Toluenesulfonic acid, trifluoroacetic acid, methyl tosylate, HCl, HBr, HI, H—Br, hydrocarbon-Br, such as, C₁ to C₂₂—Br, or a combination thereof, can be utilized as an initiator. Polymers prepared herein can be mixed together at pre-determined ratios.

The initiator can comprise a hydrophobic electrophilic molecule, including hydrocarbons, aliphatic hydrocarbons, aromatic hydrocarbons or a combination thereof, along with a halide functional group, such as, alkyl halides, aralkyl halides, acyl halides or combinations thereof. Examples of such compounds can include monofunctional initiators, such as, hydrocarbons containing from 1 to about 22 hydrocarbons with either saturated or unsaturated chemical bonds, such as, methyl iodide/bromide/chloride, ethyl iodide/bromide/chloride, 1-iodo/bromo/chloro butane, 1-iodo/bromo/chloro hexane, 1-iodo/bromo/chloro dodecane, 1-iodo/bromo/chloro octadodecane, benzyl iodide/bromide/chloride and so on. Other initiators can include allyl bromides/chlorides. Acyl halides, such as, acyl bromide/chloride, benzoyl bromide/chloride and tosyl group-containing compounds, such as, p-toluenesulfonic acid, methyl tosylate and other tosylate esters can also be used. Any one or more initiators can be used in combination. In some cases, the initiator can also comprise a hydrophilic moiety comprises proton/H containing molecules, such as, p-Toluenesulfonic acid, trifluoroacetic acid, methyl tosylate, HCl, HBr. HI, or a combination thereof.

During polymerization, an initiator can be used to start polymerization. When used, various molar ratios of monomer to initiator can be used to obtain particular polymers. The particular polymers can have differing properties, such as, molecular weight, size of branching and other properties including those unexpectedly discovered by Applicants as disclosed herein. In some cases, suitable monomer to initiator molar ratios can be 20:1 to 100:1 including any and all ratios within the range, such as, 20:1, 25:1, 30:1, 35:1, 40:1, 45:1, 50:1, 55:1, 60:1, 65:1, 70:1, 75:1, 80:1, 85:1, 90:1, 95:1 and 100:1 including 20;1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54;1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, and so on, meaning that a molar ratio of monomer to initiator in a range specified above is used to produce a polymer of choice. In some cases, the polyoxazoline disclosed herein can comprise a molar ratio of monomer to initiator in a range of from 50:1 to 80:1, meaning that a molar ratio of monomer to initiator in a range of from 50:1 to 80:1, including any and all ratios within the range, can be used to produce a polymer of choice.

The polymer can be prepared with monomers and an initiator as described herein and in earlier PCT Publication No.: WO2014/123791, herein incorporated by reference in entirety.

Hydrogen modified randomly branched PEOX polymer having a certain monomer to initiator molar ratio in a range of from 20:1 to 100:1 can be prepared as described above with an initiator selected from hydrophilic moiety comprises proton/H containing molecules, such as, p-Toluenesulfonic acid, trifluoroacetic acid, methyl tosylate, HCl, HBr, HI, or a combination thereof.

Hydrocarbon C₁ to (CH₃(CH₂)₂₁)-modified randomly branched PEOX polymer having monomer to initiator molar ratio in a range of from 20:1 to 100:1 can be prepared as described above with an initiator selected from CH₃—Br, (CH₃(CH₂))—Br, (CH₃(CH₂)₂)—Br, (CH₃(CH₂)₃)—Br, (CH₃(CH₂)₄)—Br, (CH₃(CH₂)₅)—Br, (CH₃(CH₂)₆)—Br, (CH₃(CH₂)₇)—Br, (CH₃(CH₂)₈)—Br, (CH₃(CH₂)₉)—Br, (CH₃(CH₂)₁₀)—Br, (CH₃(CH₂)₁₂)—Br, (CH₃(CH₂)₁₂)—Br(CH₃(CH₂)₁₃)—Br, (CH₃(CH₂)₁₄)—Br, (CH₃(CH₂)₁₅)—Br, (CH₃(CH₂)₁₆)—Br, (CH₃(CH₂)₁₇)—Br, (CH₃(CH₂)₁₈)—Br, (CH₃(CH₂)₁₉)—Br, (CH₃(CH₂)₂₀)—Br, and (CH₃(CH₂)₂₁)—Br. A mixture of the initiators can also be suitable.

In some cases, a polymer comprising a mixture of hydrocarbon, such as, C₁ to (CH₃(CH₂)₂₁)-modified first terminal group and H modified first terminal group can be produced by mixing the Hydrogen modified randomly branched PEOX polymer and the Hydrocarbon C₁ to (CH₃(CH₂)₂₁)-modified randomly branched PEOX polymer prepared above at a predetermined ratio.

In some cases, the polymer can comprise in a range of from 1% to 99% of the hydrocarbon, such as, C₁ to (CH₃(CH₂)₂₁)-modified first terminal group and in a range of from 1% to 99% of H modified first terminal group.

In some cases, the first terminal group can comprise in a ratio of H to hydrophobic moiety having C₁ to C₂₂ hydrocarbon, such as, (CH₃(CH₂)₁₇)— in a range of from 0.01:1 to 100:1. In some cases, the first terminal group can comprise in a ratio of H to hydrophobic moiety having C₁ to C₂₂ hydrocarbon, such as, (CH₃(CH₂)₁₇)— in arrange of from 0.1:1 to 5:1.

Polymers comprising a mixture of hydrocarbon (CH₃(CH₂)₁₇)-modified first terminals and H modified first terminals can be referred to as “H/C₁₈PEOXABP”. Polymers having specific initiator molar ratio, such as, 60:1, 70:1, 80:1, and so on, can be referred to as “H/C₁₈PEOXABP60”, “H/C₁₈PEOXABP70”, “H/C₁₈PEOXABP80”, and so on, respectively. The polymer disclosed above and hereafter can be suitable and can comprise a linear polymer, a branched polymer, a symmetrically branched polymer, an asymmetrically branched polymer, a dendrimer, a dendrigraft polymer, a comb-branched polymer, a star-branched polymer, or a combination thereof. The polymer is water soluble. In examples, the polymer can be dissolved in water to produce, for example, a 12% weight percent or higher water solution.

The second terminal group can comprise a group modified by an ammonia, a derivative of ammonia, an ethylenediamine (EDA), a derivative of ethylenediamine, a piperazine, a derivative of piperazine, tris(2 aminoethyl)amine, 4-(aminomethyl)piperidine, 1,3-diaminopropane, 2,2′-(ethylenedioxy)bis(ethylamine), diethylenetriamine, 1,4,7,10-tetraazacyclododecane, hexamethylenediamine, triethylenetetramine, 1,8-diaminooctane, or a combination thereof. In yet another example, the second terminal group can comprise a group modified by an ethylenediamine (EDA), a derivative of ethylenediamine, or a combination thereof. Any derivative of ethylenediamine disclosed herein can be suitable. The polymer can have a reaction challenge molar ratio of polyoxazoline reactive chain end to EDA in a range of from 1:1 to 1:100. The polymer can have a reaction challenge molar ratio of polyoxazoline reactive chain end to EDA in a range of from 1:1 to 1:100 in one example, 1:2 to 1:100 in another example, 1:2 to 1:50 in yet another example, 1:2 to 1:40 in yet another example, 1:2 to 1:30 in a further example, 1:2 to 1:20 in yet another example, 1:2 to 1:15 in yet another example, and 1:5 to 1:15 in a further example. In further examples, a polymer can have a reaction challenge molar ratio of polyoxazoline reactive chain end to EDA at a ratio of about 1:10. The EDA modified polyoxazoline disclosed herein can provide functional groups that can have pH-dependent changes in polymer charge as disclosed herein. In some cases, a pharmaceutical composition disclosed herein can comprise a polymer that can have a molar ratio of polyoxazoline reactive chain end to EDA of about 1:10. In some cases, in a range of from 1% to 99%, 1% to 90%, 1% to 80%, 1% to 70%, 1% to 60%, 1% to 50%, 1% to 40%, 1% to 30%, 1% to 20%, 1% to 10%, 1% to 5%, 1% to 4%, 1% to 3%, 1% to 2%, of the second terminal group can comprise a group modified by EDA. In some cases, in a range of from 1% to 99%, 1% to 90%, 1% to 80%, 1% to 70%, 1% to 60%, 1% to 50%, 1% to 40%, 1% to 30%, 1% to 20%, 1% to 10%, 1% to 5%, 1% to 4%, 1% to 3%, 1% to 2%, of the second terminal group can comprise a primary amine.

In some cases, the second terminal group can comprise a group modified by an amine, amide, imine, imide, carboxyl, hydroxyl, ester, ether, acetate, phosphate, ketone, aldehyde, sulfonate, or a combination thereof, with a proviso that in a range of from 0.01% to 100%, 0.1% to 100%, 1% to 100%, 5% to 100%, 10% to 100%, 15% to 100%, 20% to 100%, 30% to 100%, 40% to 100%, 50% to 100%, 60% to 100%, 70% to 100%, 80% to 100%, 90% to 100%, 95% to 100%, 99% to 100%, of the second terminal group can be free from primary amine. In some cases, about 1%, 5%, 10%, 15%, 20%, 25%, 30%, 35%, 40%, 45%, 50%, 55%, 60%, 65%, 70%, 75%, 80%, 85%, 90%, 95% or 100%, of the second terminal group can be free from primary amine. In some cases, about 50% to 100% of the second terminal group can be free from primary amine. In some cases, about 75% to 100% of the second terminal group can be free from primary amine. Yet in some cases, about 90% to 100% of the second terminal group can be free from primary amine.

In some cases, 100% of the second terminal group of the polymer can comprise a group modified with hydroxyl group. In some cases, as disclosed herein, CH₃(CH₂)₁₇—Br can be utilized as an initiator for 2-ethyloxazoline polymerization through a cationic ring opening process to generate a randomly branched polymer, followed by, for example, dissolving the randomly branched polymer in water to produce a second terminal modified by a hydroxyl group. In some cases, an initiator selected from hydrophilic moiety comprises proton/H containing molecules, such as, p-Toluenesulfonic acid, trifluoroacetic acid, methyl tosylate, HCl, HBr, HI, or a combination thereof, can be utilized as an initiator for 2-ethyloxazoline polymerization through a cationic ring opening process to generate a randomly branched polymer, followed by, for example, dissolving the randomly branched polymer in water to produce a second terminal modified by a hydroxyl group. In some cases, about 100% of the second terminal group can comprise hydroxyl group. In some cases, about 100% of the second terminal group can be free from primary amine.

In some cases, the pharmaceutical composition can have a pH value in a range of from about 3.0 to about 10.0, and wherein in a range of from 1% to 100% of the second terminal group is free from primary amine. In some cases, the pharmaceutical composition can have a pH value in a range of from about 3.0 to about 6.9, 4.0 to about 6.9, or 5.6 to about 6.9, and wherein about 100% of the second terminal group is free from primary amine, i.e., 0% of the second terminal group contains primary amine. In some cases, in a range of from 1% to 100% of the second terminal group can comprise hydroxyl group, percentage based on the total number of the second terminal groups.

Polymers H/C₁₈PEOXABP having a hydroxyl group as the second terminal group can be referred to as H/C₁₈PEOXABP-OH. Polymers having a specific initiator molar ratio, such as, 60:1, 70:1, 80:1, and so on, can be referred to as “H/C₁₈PEOXABP60-OH”, “H/C₁₈PEOXABP70-OH”, “H/C₁₈PEOXABP80-OH”, and so on, respectively. Polymers H/C₁₈PEOXABP having an amine group as the second terminal group can be referred to as H/C₁₈PEOXABP-NH₂. Polymers having a specific initiator molar ratio, such as, 60:1, 70:1, 80:1, and so on, can be referred to as “H/C₁₈PEOXABP60-NH₂”, “H/C₁₈PEOXABP70-NH₂”, “H/C₁₈PEOXABP80-NH₂”, and so on, respectively. In some cases, mixtures of the polymers disclosed herein can be suitable.

Not wishing to be bound by a particular theory or mechanism, Applicants believe that primary amine at a certain level may interact with some bioactive agent, for example, rapamycin, leading to the degradation of the bioactive agent. Applicants discovered that polymers without primary amine or having various percentages of primary amine on the second terminal group can be used to modulate properties, such as, stability or degradation, of a certain bioactive agent, such as, rapamycin, providing an additional approach in optimizing formulations of the pharmaceutical composition.

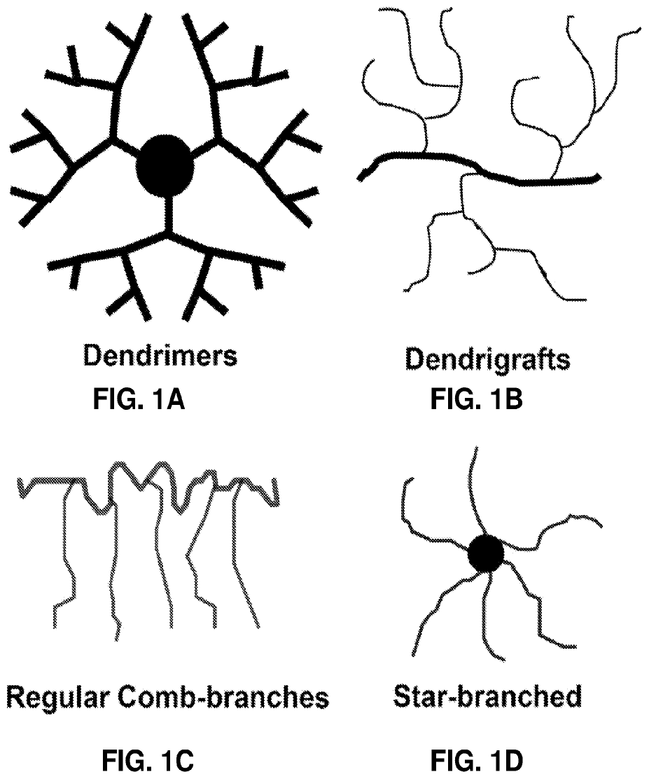

In any of pharmaceutical compositions disclosed above and hereafter, the polymer can comprise a polyoxazoline (POX) that comprises a linear portion, a branched portion, or a combination thereof. The polymer can comprise a plurality of linear portions joined together in one example, one or more linear portions joined with one or more branched portions in another example, one or more branched portions joined together in yet another example, such as, those schematically depicted in FIG. 1A through FIG. 10B. Each of the linear portions can be, independently, of various lengths, various modifications, or a combination thereof. Each of the branched portions can be, independently, of various lengths, number of branches, various modifications, or a combination thereof.

The polyoxazoline (POX) can comprise poly(2-oxazoline), poly(2-substituted oxazoline) that comprises poly(2-methyloxazoline), poly(2-ethyloxazoline), poly(2-propyloxazoline), poly(isopropyloxazoline) (PiPOX), or a combination thereof. The POX can comprise poly(2-methyloxazoline) (PMOX) in one example, poly(2-ethyloxazoline) (PEOX) in another example, poly(2-propyloxazoline) (PPOX) in yet another example, poly(isopropyloxazoline) (PiPOX) in yet another example, or a combination of two or more of the poly(2-substituted oxazoline)s in yet a further example, wherein the two or more of the poly(2-substituted oxazoline)s can be a repeating unit, also referred to as complex monomer, in the polyoxazoline polymer. The polyoxazoline (POX) is hydrophilic. The polyoxazoline (POX) can be free from monomers, either simple or complex monomers, having hydrophobic side chains, such as, those having 4 or more carbons (C₄ and above).

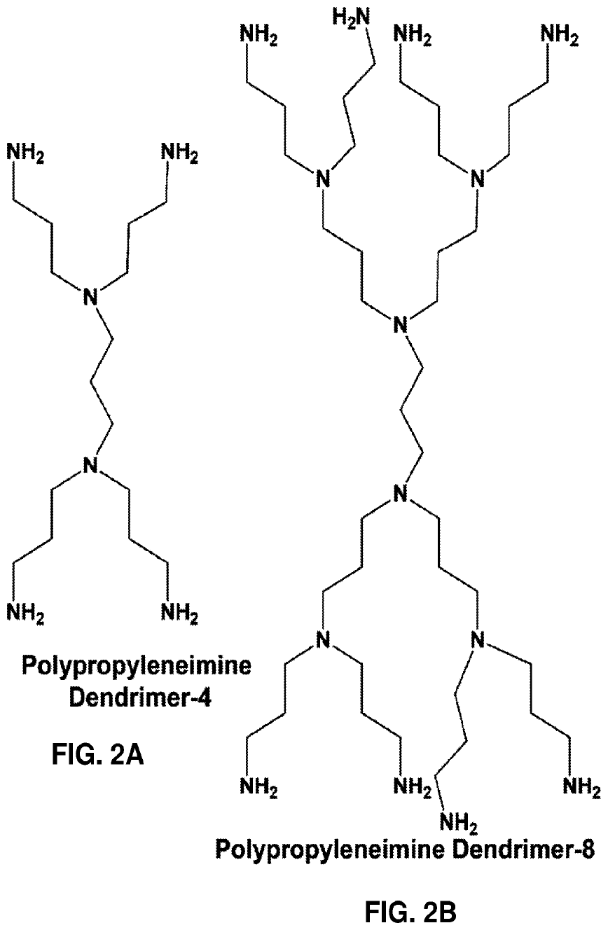

Some of examples of symmetrically branched polymers (SBP) are schematically depicted in FIG. 1A-FIG. 1D and FIG. 2A-FIG. 2B with symmetric branches, wherein all the homopolymers of interest possess a core and exhibit symmetric branch junctures consisting either of terminal or chain branches throughout the homopolymer. The functional groups are present predominantly at the exterior of the polymer.

The modified SBP's can be obtained, for example, through chemically linking functional groups on, for example, symmetrically branched PAMAM or PPI dendrimers, commercially available from Aldrich, polyether dendrimers, polyester dendrimers, comb-branched/star-branched polymers, such as, those containing PEO, PEG, PMOX or PEOX, polystyrene, and comb-branched dendrigrafts, such as, those containing PEOX, PMOX or PEI. The synthetic procedures for making such SBP's/dendrimers are known and described above and hereafter.

In some cases, the higher branching densities of SBP's can render the polymers molecularly compact with a well-defined interior void space, which makes such molecules suitable as a carrier for water insoluble or poorly water soluble drugs, such as, rapamycin, entrapped or encased, therein.

The surface modifications can enhance the properties and uses of the resulting modified SBP's. For example, with suitable modification, a water insoluble SBP can become water soluble, while an SBP with a high charge density can be modified to carry very low or no charge on the polymer or at the polymer surface. On the other hand, a water soluble SBP can be modified with hydrophobic surface groups to enhance the ability to solubilize water insoluble or poorly water soluble drugs at the surface or in the interior thereof. Modification can occur at any site of a polymer, for example, at a terminus, a branch, a backbone residue and so on.

In one embodiment of the instant disclosure, the SBP (for example, either a symmetrically branched PEI dendrimer, a PPI dendrimer, a PAMAM dendrimer or a symmetrically branched PEI dendrigraft) can be modified with different kinds of, for example, primary amine groups through, for example, Michael addition or an addition of acrylic esters onto amine groups of the homopolymer. Thus, for example, through a Michael addition reaction, methyl acrylate can be introduced onto the primary and/or secondary amino groups of PEI, PPI and polylysine (PLL) homopolymers. The ester groups then can be derivatized further, for example, by an amidation reaction. Thus, for example, such an amidation reaction with, for example, ethylenediamine (EDA), can yield the addition of an amino group at the terminus of the newly formed branch. Other modifications to the homopolymer can be made using known chemistries, for example, as provided in “Poly(amines) and Poly(ammonium salts),” in “Handbook of Polymer Synthesis,” (Part A), Kricheldorf ed., New York, Marcel Dekker, 1994; and “Dendrimers and Other Dendritic Polymers” Frechet & Tomalia, eds., John Wiley & Sons, Ltd., 2001. Derivatives of EDA also can be used and include any molecular entity that comprises a reactive EDA, a substituted EDA or, for example, other members of the polyethylene amine family, such as, diethylenetriamine, triethylenetetramine, tetraethylenepentamine, pentaethylenehexamine, and so on including polyethylene amine, tetramethylethylenediamine and so on. The amidation reaction with, for example, ethylenediamine (EDA), can also modify polymer charge density at the terminus of the newly formed branch. As disclosed herein, polymer having such amidation groups can have pH-dependent change in charge leading to change in pH-dependent polymer charge density.

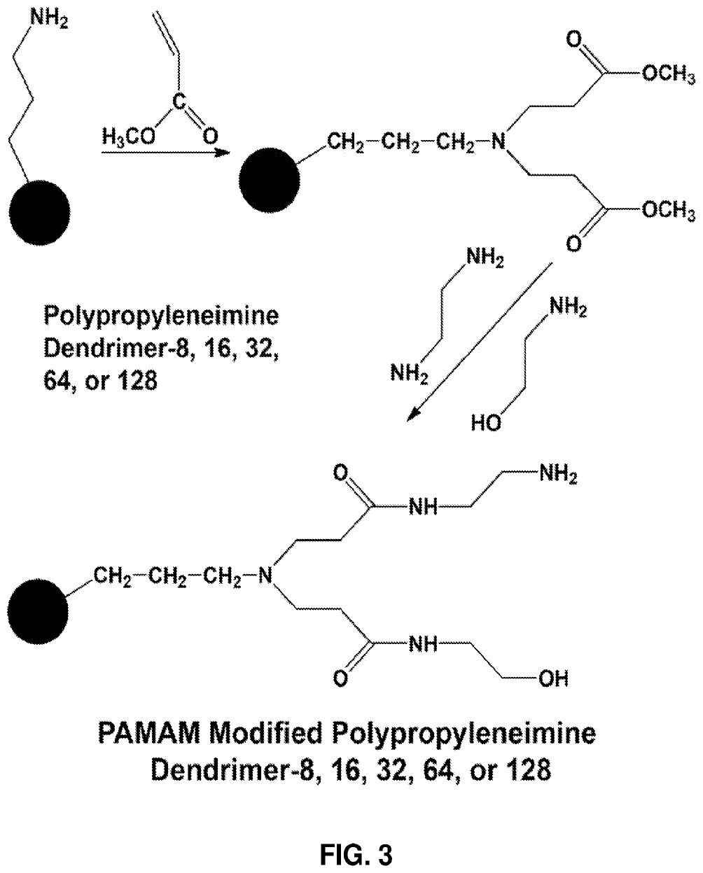

n some embodiments, a modification can comprise a moiety that contributes to or enhances hydrophobicity of a polymer or a portion thereof. For example, hydrophobic functional groups, such as, aliphatic chains including hydrocarbon chains comprising 1 to about 22 carbons that can be saturated or unsaturated, linear, cyclic or branched, aromatic structures (e.g. containing one or more aromatic rings, which may be fused) or combinations thereof, can be used as a modifying agent and added to a polymer as taught herein practicing chemistries as provided herein. On such addition, a modified SBP, such as, a modified PEI, PPI, PAMAM dendrimer or PEI dendrigraft, can be formed. An example of PAMAM modified PPI dendrimer is shown in FIG. 3. As an extension of the SBP, such as, PPI and PEI, the resulting modified SBP also is symmetrically branched. Depending on the solvent environment (i.e. pH or polarity), the surface functional groups can carry different charge and/or charge density, and/or hydrophobic groups. The molecular shape and surface functional group locations (i.e., surface functional group back folding) then can be tuned further, based on those characteristic properties.

In another embodiment of the disclosure, the modified SBP's can be produced using any of a variety of synthetic schemes that, for example, are known to be amenable to reaction with a suitable site on the homopolymer. Moreover, any of a variety of reagents can be used in a synthetic scheme of choice to yield any of a variety of modifications or additions to the homopolymer backbone. Thus, for example, in the case of the Michael addition reaction to an amine described above, the addition of any of a variety of substituents can be used, for example, at the alkylation stage, using for example, any of a variety of acrylate reagents, such as, an acrylate comprising a hydrocarbon substituent, such as, saturated or unsaturated hydrocarbons comprising 1 to about 22 carbons, which may be substituted, aliphatic, aromatic, ringed, saturated at one or more bonds or a combination thereof. Thus, suitable reactants include, methyl acrylate, ethyl acrylate, propyl acrylate, butyl acrylate, pentyl acrylate, hexyl acrylate, heptyl acrylate, octyl acrylate, nonyl acrylate, decyl acrylate, undecyl acrylate, dodecyl acrylate and so on, and mixtures thereof. Similarly, at the amidation stage in the example exemplified above, any of a variety of amines can be used. For example, EDA, monoethanolamine, tris(hydroxymethyl)aminomethane, alkyl amine, allyl amine or any amino-modified polymer, including those comprising PEG, PEO, perfluoropolymers, polystyrene, polyethylene, polydimethylsiloxane, polyacrylate, polymethylmethacrylate and the like, and mixtures thereof, can be used.

Such a synthetic strategy would allow not only symmetric growth of the molecule, where more branches with different chemical compositions can be introduced, but also the addition of multiple functional groups at the exterior of the polymer structure. The precursor homopolymer can be modified, and continuously, using the same or a different synthetic process until the desired SBPs with appropriate molecular weight and functional groups are attained. In addition, the hydrophobic and hydrophilic properties, as well as charge densities of such polymers, can be tailored to fit specific application needs using appropriate monomers for constructing the homopolymer and suitable modification reactions.

In another embodiment of the disclosure, if a divergent synthetic procedure is used, the chain end of symmetrically star-branched or comb-branched homopolymer, such as, poly(2-oxazoline) or poly(2-substituted oxazoline), including, for example, poly(2-methyloxazoline), poly(2-ethyloxazoline), poly(2-propyloxazoline) and poly(2-butyloxazoline, etc.), PEI, PEO/glycol, polyvinylpyrrolidone (PVP), polyphosphate, polyvinyl alcohol (PVA) or polystyrene, can be modified with another small molecule or polymer to generate various functional groups at the homopolymeric chain ends including a primary, secondary ortertiary amine, carboxylate, hydroxyl, aliphatic (e.g., hydrocarbon chain), aromatic, fluoroalkyl, aryl, PEG, PEO, acetate, amide and/or ester groups. Alternatively, various initiators also can be utilized so that the same type of functional groups can be introduced at the chain end if a convergent synthetic approach is utilized (“Dendritic Molecules,” Newkome et al., eds., VCH, Weinheim, 1996; “Dendrimers and Other Dendritic Polymers,” Frechet & Tomalia, eds., John Wiley & Sons, Ltd., 2001; and J. Macromol. Sci. Chem. A9(5), pp. 703-727 (1975)).

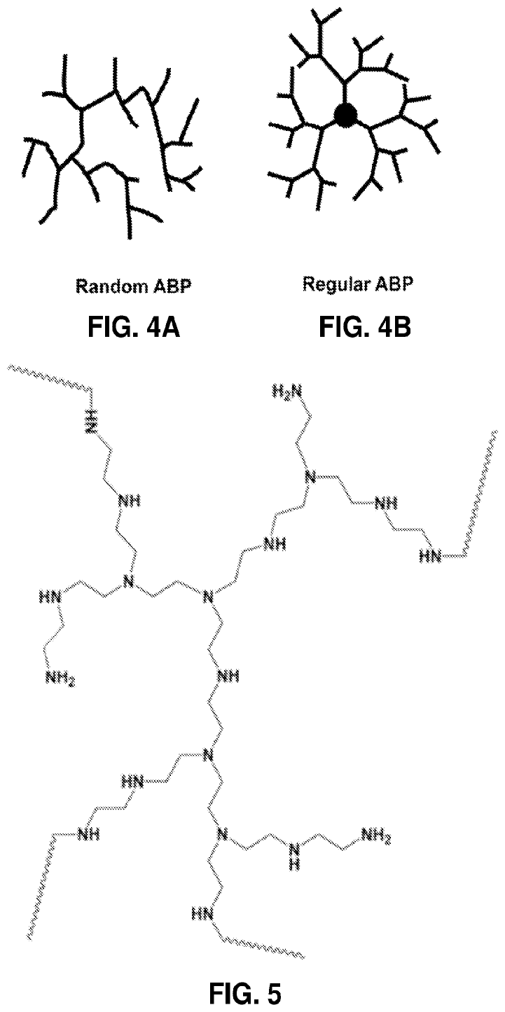

Some examples of asymmetrically branched polymers (ABP) are schematically depicted in FIG. 4A-FIG. 4B with asymmetric branches, wherein some of the polymers of interest possess no core and exhibit asymmetrical branch junctures consisting of both chain and terminal branches throughout the entire homopolymer. The junctional groups often are present both at the exterior and in the interior. However, when a larger functional group (e.g., a large hydrophobic or hydrophilic group) is used, the functional groups often can be attached preferentially and perhaps necessarily at the exterior of the ABP, for example, possibly due to steric effects. Therefore, such surface modified branched polymers (MBP) can be utilized for solubilization of or nanoaggregate formation with a water insoluble or poorly water soluble drug.

The modified ABP's can be obtained, for example, through chemically linking functional groups on regular ABP's, such as, polylysine (e.g., branched PLL), on random ABP's, such as, PEI's (commercially available from Aldrich, Polysciences, or BASF under the trade name, Lupasol®) or polyoxazolines, which can be prepared according to the procedure of Litt (J. Macromol. Sci. Chem. A9(5), pp. 703-727 (1975)). Other ABP's can include, but are not limited to, polyacrylamides, polyphosphates, PVP's, PVA's etc. The random asymmetrically branched PEI's can be produced primarily through cationic ring opening polymerization of ring-strained cyclic imine monomers, such as, aziridines (ethyleneimine) and azetidines (propyleneimine), with Lewis or Bronsted acids as initiators (Dernier et al., “Ethylenediamine and Other Aziridines,” Academic Press, New York, (1969); and Pell, J. Chem. Soc. 71 (1959)). Since many of the methods are essentially one-pot processes, large quantities of random ABP's can be produced readily.

The synthetic processes for making ABP's often generate various branch junctures within the macromolecule. In other words, a mixture of terminal and chain branch junctures is distributed throughout the molecular structure. The branching densities of the random ABP's can be lower, and the molecular structure can be more open when compared with dendrimers and dendrigrafts. Although the branch pattern is random, the average ratio of primary, secondary and tertiary amine groups can be relatively consistent with a ratio of about 1:2:1, as described by Dick et al., J. Macromol. Sci. Chem., A4 (6), 1301-1314 (1970) and Lukovkin, Eur. Polym. J. 9, 559(1973). In one example, the polymer disclosed herein can comprise a ratio of primary, secondary and tertiary amine groups of about 1:2:1.

The presence of the branch junctures can make the random ABP's, such as, asymmetrically branched PEI's, form macromolecules with a possible spherical, ovoid or similar configuration. Within the globular structure, there are various sizes of pockets formed from the imperfect branch junctures at the interior of the macromolecule. Unlike dendrimers and dendrigrafts where interior pockets are always located around the center core of the molecule, the pockets of random ABP's are spread unevenly throughout the entire molecule. As a result, random ABP's possess both exterior and unevenly distributed interior functional groups that can be reacted further with a variety of molecules, thus forming new macromolecular architectures, a modified random ABP of interest.

Although having a core, the functional groups of the regular ABP can also be distributed both at the exterior and in the interior, which is very similar to the random ABP. One such homopolymer is PLL, which can be made as described in U.S. Pat. Nos. 4,289,872; 4,360,646; and 4,410,688, each incorporated by reference in entirety. Such homopolymers also can be modified in a manner similar as that for random ABP's, as taught herein, and as known in the art.

In an embodiment of the disclosure, the ABP (for example, either a random asymmetrically branched PEI or a regular asymmetrically branched PLL) is modified with different kinds of primary amine and/or secondary amine groups through, for example, Michael addition or an addition of acrylic esters onto amines of the polymer, for example, PEI and PLL homopolymers. The ester groups then can be further derivatized, for example, by an amidation reaction. Thus, for example, such an amidation reaction with, for example, EDA, can yield the addition of an amino group at the terminus of the newly formed branch. Other modifications to the polymer can be made using known chemistries, for example, as provided in aforementioned “Poly(amines) and Poly(ammonium salts)”. On such addition, a modified ABP, such as, a modified PEI or PLL homopolymer, is formed. As an extension of the ABP, such as, PEI and PLL, the resulting modified ABP also is branched, asymmetrically. Depending on the solvent environment (i.e. pH or polarity), the surface functional groups can carry different charge and charge density. The molecular shape and functional group locations (i.e., functional group back folding) then can be further tuned, based on those characteristic properties.

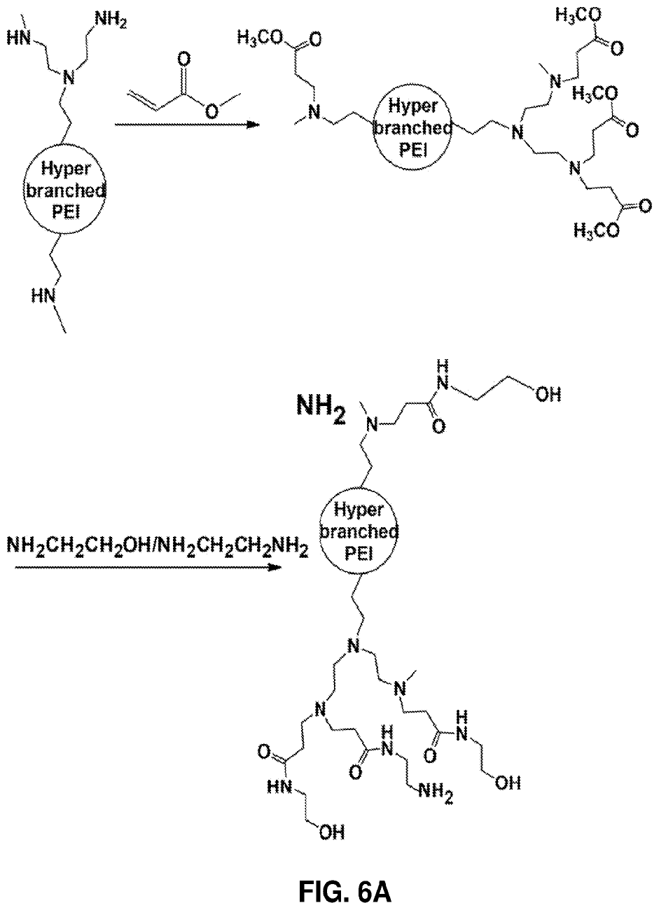

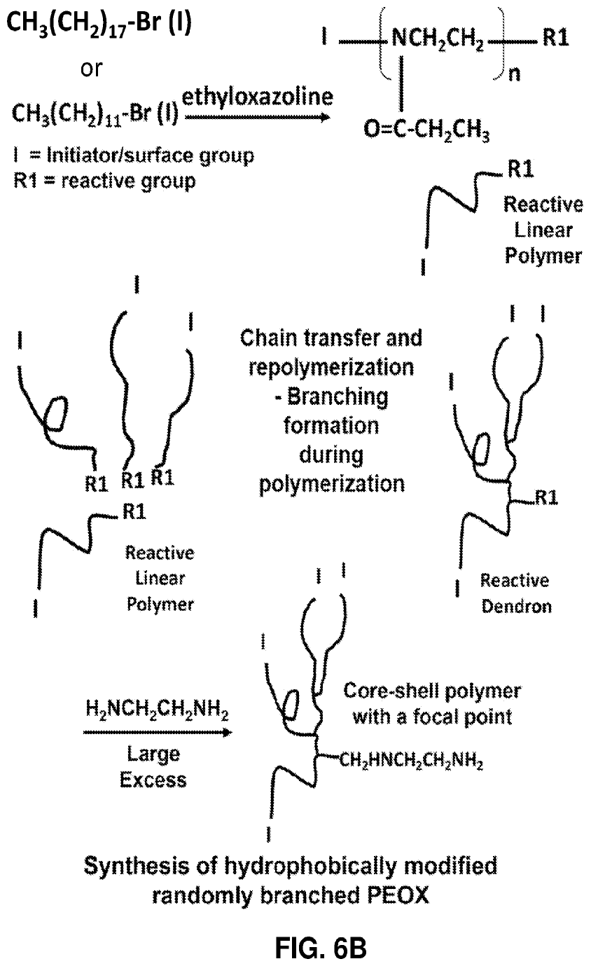

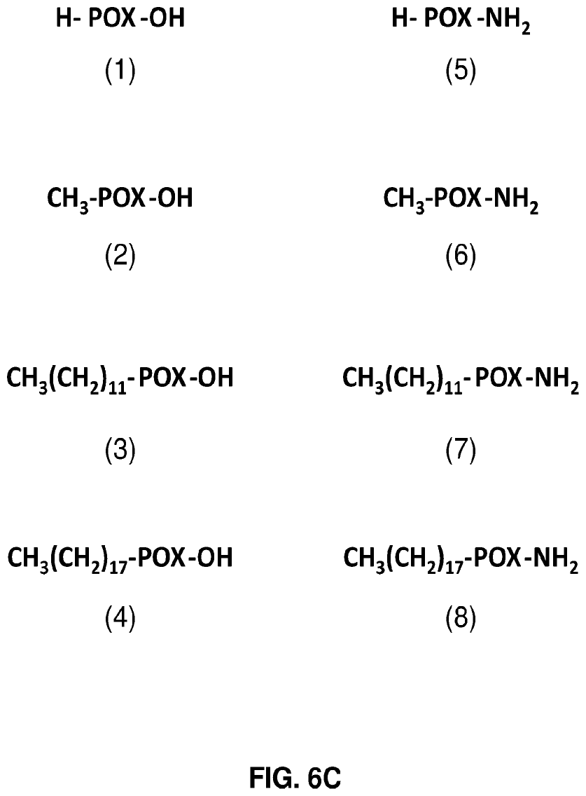

In another embodiment, the modified ABP's can be produced using any of a variety of synthetic schemes that, for example, are known to be amenable to reaction with a suitable site on the homopolymer. Moreover, any of a variety of reagents can be used in a synthetic scheme of choice to yield any of a variety of modifications or additions to the polymer backbone. Thus, for example, in the case of the Michael addition reaction to an amine described above, the addition of any of a variety of substituents can be used at the alkylation stage, as provided hereinabove, for example, with an acrylate, which can comprise a saturated or unsaturated hydrocarbon, such as, one comprising one carbon to about 22 carbons, which may be aliphatic, branched, saturated, aromatic, ringed or combination thereof. The hydrocarbon can have 2 to 22 carbons in one example, 4 to 22 carbons in another example, 6 to 22 carbons in yet another example, 7 to 22 carbons in yet another example, 8 to 22 carbons in yet another example, 10 to 22 carbons in yet another example, 12 to 22 carbons in yet another example, 14 to 22 carbons in yet another example, 16 to 22 carbons in yet another example, 18 to 22 carbons in a further example, and 20 to 22 carbons in yet a further example. In one particular example, the first terminal group can comprise 18 carbons, such as, a (CH₃(CH₂)₁₇)-group. Suitable reactants include methyl acrylate, ethyl acrylate, propyl, acrylate, butyl acrylate, pentyl acrylate, hexyl acrylate, heptyl acrylate, octyl acrylate, nonyl acrylate, decyl acrylate, undecyl acrylate, dodecyl acrylate and the like, and mixtures thereof. Similarly, at the amidation stage in the example exemplified above, any of a variety of amines can be used in the methods provided herein and known in the art. For example, EDA, monoethanolamine, tris(hydroxymethyl)aminomethane, alkyl amine, allyl amine or any amino-modified polymers, including PEG, perfluoropolymers, polystyrene, polyethylene, polydimethylsiloxane, polyacrylate, polymethylmethacrylate and the like, and mixtures thereof, can be used. In addition, the linking of the hydrophobic groups, including aliphatic (e.g., hydrocarbons from C₁ to about C₂₂) groups, aromatic groups, polyethylene polymers, polystyrene polymers, perfluoropolymers, polydimethylsiloxanes, polyacrylates, polymethylmethacrylates, as well as, hydrophilic groups, including a OH group, hydrophilic polymers, such as, PEOX, PEG, PEO etc. to a modified ABP can be achieved by using, for example, epoxy reactions, amidation reactions, Michael addition reactions, including using a —SH or an —NH₂ group reacted with maleimide, aldehyde/ketone-amine/hydrazide coupling reactions, iodo/iodoacetyl-SH coupling reactions, hydroxylamine-aldehyde/ketone coupling reactions etc. Such synthetic strategies allow not only asymmetric growth of the molecule, where more pockets are introduced, but also the addition of multiple functional groups at both the interior and the exterior of the structure. The homopolymer can be modified further using the same or a different synthetic process until the desired ABP's with appropriate molecular weight and functional groups are attained. In addition, the hydrophobic and hydrophilic properties, as well as charge density of such homopolymers, can be tailored to fit specific application needs using appropriate monomers for constructing the homopolymer and suitable modification reactions. An example of a modified ABP is shown in FIG. 5. A modified hyperbranched PEI is shown in FIG. 6A.

In another embodiment of the disclosure, a focal point (merged from various reactive chain ends during a convergent synthesis) of a random ABP, such as, POX, can be terminated or reacted with another small molecule to generate various functional groups at the homopolymeric chain ends, including primary, secondary or tertiary amines, carboxylate, hydroxyl, alkyl, fluoroalkyl, aryl, PEG, acetate, amide and/or ester groups. Alternatively, various initiators also can be utilized so that the same type of functional group can be introduced at the surface groups where a polymerization begins during a convergent synthesis (J. Macromol. Sci. Chem. A9 (5), pp. 703-727(1975)),

An alkyl surface-modified, randomly branched poly(2-ethyloxazoline) with a primary amine group at the focal point of the branched polymer can be prepared using the Litt and Warakomski procedures, supra. For example, CH₃(CH₂)₁₇—Br can be utilized as an initiator for 2-ethyloxazoline polymerization through a cationic ring opening process to generate a randomly branched polymer, followed by quenching with N-ten-butyloxycarbonylpiperazine (N-Boc-piperazine) or EDA. The termination with a large excess of EDA allows the hydrophobically modified branched poly(2-ethyloxazoline) polymer to be functionalized with a primary amine group at the focal point (FIG. 6B). Alternatively, N-Boc-piperazine-terminated hydrophobically-modified branched poly(2-ethyloxazoline) polymer also can be deprotected to generate a free amino group at the focal point. In some cases, the polymer can comprise a modified branched poly(2-ethyloxazoline) functionalized with primary, secondary or tertiary amines, carboxylate, hydroxyl, alkyl, fluoroalkyl, aryl, PEG, acetate, amide or ester groups at a focal point of the polymer where two or more reactive chain ends merged during a convergent synthesis.

In some cases, an alkyl surface-modified, randomly branched poly(2-ethyloxazoline) can have a hydroxyl group at the focal point of the branched polymer. For example, the focal point of the polymer can be hydrolyzed to, for example, a hydroxyl group on dissolving in water (e.g., containing, for example, 1 N Na₂CO₃).

In some cases, the focal point of the polymer mentioned herein can comprise a second terminal group modified with a hydrophilic moiety.

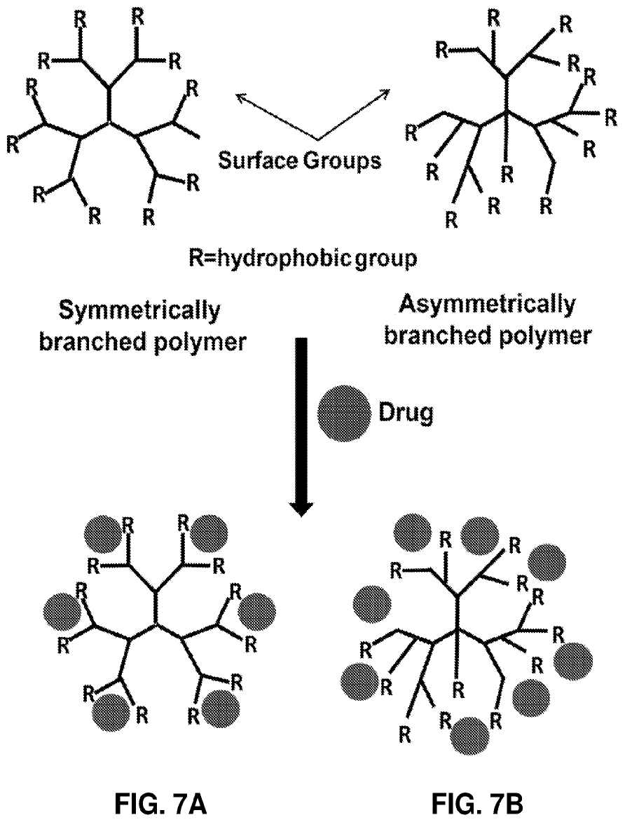

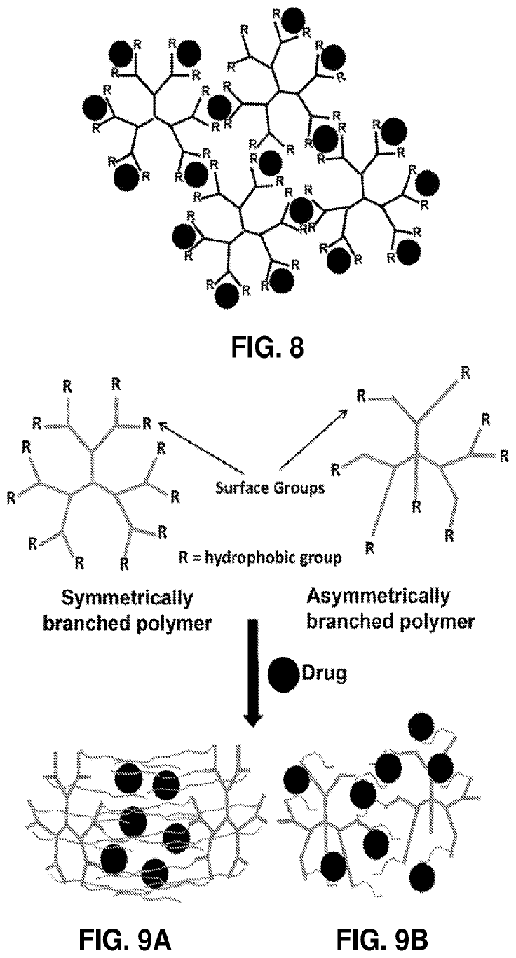

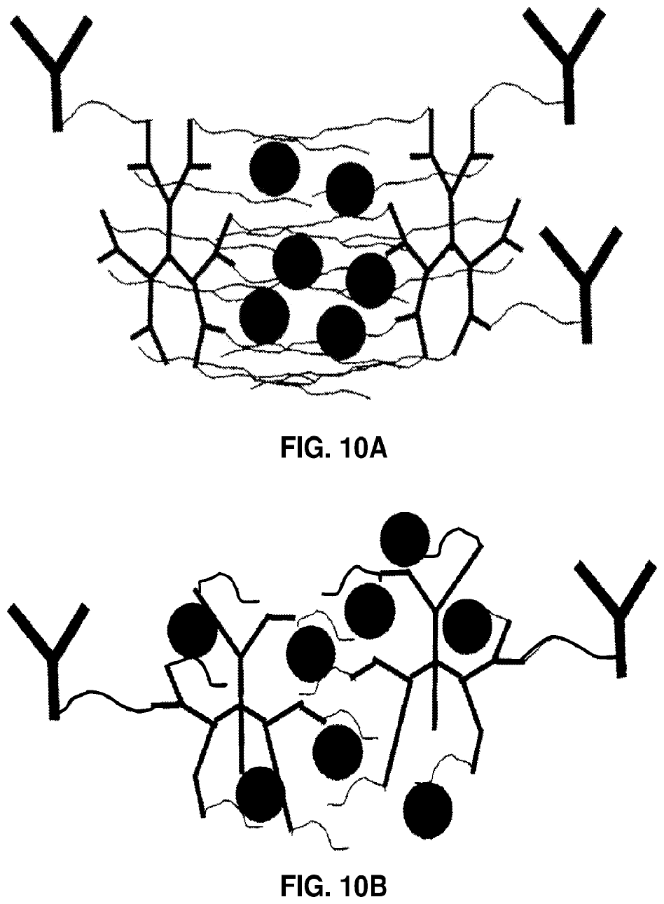

While the introduction of a primary amine group to a hydrophobically-modified branched poly(2-oxazoline) homopolymer enhances drug solubility and produces bioactive agent-induced nanoaggregates (such as, shown in FIG. 7A-FIG. 7B, FIG. 8, FIG. 9A-FIG. 9B), the primary amine group also allows the attachment of various targeting groups, such as, an antibody, antigen-binding portion thereof, an antigen or a member of a binding pair, such as, to the hydrophobically modified branched poly(2-oxazoline) polymer (FIG. 10A-FIG. 10B). This can be particularly useful prior to mixing the polymer and a bioactive agent, such as, rapamycin. Such nanoaggregates or nanoparticles containing such targeting groups and modifications thereto can provide a targeting ability on the nanoaggregate with a bioactive agent, such as, rapamycin, and enable the bioactive agent to be released preferentially or solely at desired treatment locations. As mentioned above, it is preferred to have a polymer that in a range of from 1% to 100% of the second terminal group is free from primary amine when the polymer is used to mix with rapamycin to produce the pharmaceutical composition of this disclosure.

As disclosed herein, modified branched polymers (MBP), such as, hydrophobically-modified homopolymers, including SBP's, ABP's, or a combination thereof, can be used to generate an encapsulating polymer or nanocapsule for solubilizing water insoluble rapamycin. In an organic solvent environment, the hydrophilic or amphiphilic interior can be poly(2-oxazoline), poly(2-substituted oxazolines), wherein the poly(2-substituted oxazoline) can comprise poly(2-methyloxazoline), poly(2-ethyloxazoline), poly(2-propyloxazoline), poly(isopropyloxazoline) (PiPOX), or a combination thereof, PEG, PEO, polyphosphonate and the like. The hydrophobic exterior can comprise aliphatic hydrocarbons (such as, from C₁ to about C₂₂), aromatic hydrocarbons, polyethylene polymers, polystyrene polymers, perfluoropolymers, polydimethylsiloxanes, polyacrylates, polymethylmethacrylates and the like. In an aqueous environment, the reverse is true. In the drug-induced nanoaggregates in an aqueous environment, the drug molecules such as, rapamycin or other water insoluble bioactive agent can be associated with the hydrophobic groups/domains of the MBP's (FIG. 9A-FIG. 9B). The branching density (e.g., from low generation, such as, star and comb homopolymers, to high generation of dendrimers and dendrigrafts), as well as the amount of hydrophobic surface group coverage (e.g., from 0% to 100% coverage) of the branched homopolymers can affect significantly homopolymer solubility, which in turn, also affects the ability to dissolve or to adsorb/absorb rapamycin. For example, the increase in branching density and the amount of hydrophobic group coverage will make the homopolymer more compatible with, for example, rapamycin.

In further examples, the ABP's and SBP's with from about 0.1 to about 30% or more surface hydrophobic component by weight are effective at solubilizing or dispersing poorly water soluble or water insoluble compounds, such as, rapamycin. In addition, the branched homopolymers utilized, for example, a POX, a PMOX, a PEOX, a PPOX, PEO/PEG, polyacrylamides, polyphosphates, PVP's and PVA's are soluble in both water and in various organic solvents, thereby facilitating forming, for example, rapamycin-containing nanoparticles or nanoaggregates. The good water solubility along with good hydrophobic drug miscibility in an aqueous solution, with or without other organic solvents, makes such homopolymers useful for enhancing the solubility of poorly water soluble bioactive agents. For example, the homopolymers of interest simplify manufacturing processes and decrease production cost by reducing formulation steps, processing time, as well as the need to use complex and expensive equipment currently used in the pharmaceutical industry. If additional branching densities are needed, the SBP's or ABP's first can be modified with additional groups as described herein, and then, for example, attached with additional hydrophobic functional groups for enhancing, for example, rapamycin solubility.

In one example, the polymer is configured to have effective branching density, amount of hydrophobic groups at the surface of the polymer, or a combination thereof, for encapsulating a bioactive agent, such as, rapamycin, that is, in water, insoluble, to form a water soluble nanoaggregate. The effective branching density, the amount of hydrophobic groups at the surface of the polymer, or a combination thereof, can be modified as described above and hereafter.

In one example, the polymer can have hydrophobic groups, including aliphatic (e.g., hydrocarbons from C₁ to about C₂₂) groups, aromatic groups, polyethylene polymers, polystyrene polymers, perfluoropolymers, polydimethylsiloxanes, polyacrylates, polymethylmethacrylates, linked to a POX polymer including a PEOX polymer and further modified by EDA. The POX polymer can be a homopolymer polymerized from a repeating unit comprising single monomer or a repeating unit comprising two or more monomers in each repeating unit.

In some cases, the polymer can comprise asymmetrically branched polymers (ABP) or dendritic asymmetrically branched polymer, such as, asymmetrically branched PEOX formed from the initiators and monomers at ratios disclosed herein. In some cases, the polymer can comprise randomly branched poly(2-ethyloxazoline) having one or more first terminal groups, such as, the hydrophobic moiety disclosed herein, and one second terminal group positioned at the focal point of the branched polymer, such as, the modified randomly branched PEOX formed by polymerizing reactive linear PEOX polymers with chain transfer polymerization convergent synthesis as illustrated in FIG. 6B.

In some cases, the polymers can have different first terminal groups and different second terminal groups. Some examples are shown in FIG. 6C: Polymer (1)-Polymer (4) having —OH as the second terminal group and Polymer (5)-Polymer (8) having —NH₂ as the second terminal group, Polymer (1) and Polymer (5) having H as the first terminal group, Polymer (2) and Polymer (6) having —CH₃ as the first terminal group, Polymer (3) and Polymer (7) having C₁₂ as the first terminal group, and Polymer (4) and Polymer (8) having C₁₈ as the first terminal group. The polyoxazoline (POX) polymer can be a linear polymer, a branched polymer, or a polymer having a combination of one or more linear portions and one or more branched portions. The polyoxazoline (POX) can comprise poly(2-methyloxazoline), poly(2-ethyloxazoline), poly(2-propyloxazoline), poly(isopropyloxazoline) (PiPOX), or a combination thereof. Although specific first terminal groups and second terminal groups are described above, other first and second groups disclosed herein can be suitable. In some cases, the second terminal group can comprise a group modified by an amine, amide, imine, imide, carboxyl, hydroxyl, ester, ether, acetate, phosphate, ketone, aldehyde, sulfonate, or a combination thereof. The first terminal group and the second terminal group can be modified according to methods and processes known to those skilled in the art. If needed, one or more reagents, linkers or intermediates known to those skilled in the art can be used.

In any of pharmaceutical compositions disclosed above and hereafter, the polyoxazoline can comprise a molar ratio of monomer to initiator in a range of from 50:1 to 80:1.

The pharmaceutical composition can comprise additional polymers selected from ABP's, ABP's, MBP's, such as, symmetrically branched PAMAM or PPI dendrimers, polyether dendrimers, polyester dendrimers, comb-branched/star-branched polymers, such as, those containing PEO, PEG, PMOX or PEOX, polystyrene, and comb-branched dendrigrafts, such as, those containing PEOX, PMOX, PEI, polylysine (e.g., branched PLL), polyacrylamides, polyphosphates, PVP's, PVA's or a combination thereof. The random asymmetrically branched PEI's can be produced primarily through cationic ring opening polymerization of ring-strained cyclic imine monomers, such as, aziridines (ethyleneimine) and azetidines (propyleneimine), or a combination thereof. The additional polymers can be simply mixed with the nanoaggregate disclosed herein. In one example, one or more additional polymers can be mixed with the nanoaggregate after the nanoaggregate has formed.

Suitable to the pharmaceutical composition, process, method and use disclosed herein throughout this disclosure, the term “bioactive agent” refers to a substance that can be a natural or synthetic small molecule-based drug, inorganic-based drug, biological drug, natural or synthetic large molecule-based drug, modifications and/or derivatives thereof, or a combination thereof, as disclosed herein. The bioactive agent can include a natural or synthetic small molecule-based drug, inorganic-based drug, biological drug, natural or synthetic large molecule-based drug, modifications and/or derivatives thereof, or a combination thereof, wherein at least one drug is poorly water soluble or water insoluble. A drug of interest can be a small molecule, a salt thereof in which the molecule is modified to be water insoluble or poorly water soluble or can be a biological molecule which is modified to be water insoluble or poorly water soluble, particularly when a drug has improved properties, such as, improved bioavailability, less toxicity, better pharmacokinetics, or a combination thereof, in a water insoluble or poorly water soluble form. Suitable examples can include drugs which are poorly water soluble or water insoluble or can be modified to be water insoluble or poorly water soluble for an improved property.

The bioactive agent can include growth agents; AIDS adjunct agents; alcohol abuse preparations, such as, agents for treating dependence or withdrawal; Alzheimer's Disease treatment agents; Amyotrophic Lateral Sclerosis treatment agents; analgesics; anesthetics; anticonvulsants; antidiabetic agents; antidotes; antifibrosis therapy agents; antihistamines; anti-infective agents, such as, antibiotics, antivirals, antifungals, amebicides, antihelmintics, antimalarials, leprostatics and so on; antineoplastic agents; antiparkinsonian agents; antirheumatic agents; appetite stimulants; biological response modifiers; biologicals; blood modifiers, such as, anticoagulants, colony stimulating factors, hemostatics, plasma extenders, thrombin inhibitors and so on; bone metabolism regulators; cardioprotective agents; cardiovascular agents, such as, adrenergic blockers, adrenergic stimulators, angiotensin-converting enzyme (ACE) inhibitors, antiarrhythmics, antilipemic agents, calcium channel blockers, diuretics, vasopressors and so on; central nervous system (CNS) stimulants; cholinesterase inhibitors; contraceptives; fertility treatment agents; ovulation stimulators; cystic fibrosis managements agents; detoxifying agents; diagnostics; dietary supplements; dopamine receptor agonists; endometriosis management agents; enzymes; erectile dysfunction treatment agents; foot care products; gastrointestinal (GI) treatment agents, such as, antacids, antidiarrheals, antiemetics, antiflatulants, bowel evacuants, digestive enzymes, histamine receptor agonists, laxatives, proton pump inhibitors, prostaglandins and so on; Gaucher's Disease treatment agents; gout treatment agents; homeopathic remedies; skin treatments; vitamins; nutrients; hormones; hypercalcemia management treatment agents; hypocalcemia management treatment agents; immunomodulators; immunosuppressants; levocarnitine deficiency treatment agents; mast cell stabilizers; migraine treatment agents, motion sickness treatment products, such as, Benadryl and Phenergan; decongestants; antihistamines; cough suppressants; multiple sclerosis treatment agents; muscle relaxants; nasal treatment agents, such as, anti-inflammatories; smoking cessation aids; appetite suppressants; nucleoside analogs; obesity management agents; ophthalmic preparations, such as, antibiotics, antiglaucoma agents, artificial tears, lubricants and so on; sexual aids; osteoporosis treatment agents; otic preparations, such as, antiinfectives and cerumenolytics; minerals; oxytocics; parasympatholytics; parasympathomimetics; patent ductus arteriosus agents; phosphate binders; porphyria agents; prostaglandins; psychotherapeutic agents; radiopaque agents; respiratory agents, such as, antiinflammatories, antitussives, bronchodilators, decongestants, expectorants, leukotrienes antagonists, surfactants and so on; salt substitutes; sedatives; hypnotics; skin and mucous membrane treatment agents, such as, acne treatments; anorectal treatment agents, such as, hemorrhoid treatments and enemas; antiperspirants; antipruritics; antipsoriatic agents; antiseborrheic agents; burn treatment agents; cleansing agents; depigmenting agents; emollients; hair growth retardants; hair growth stimulators; keratolytics; hair problem treatment agents; mouth and throat problem treatment agents; photosensitizing agents; wart treatment agent; wound care treatment agents, or a combination thereof. The bioactive agent can also include over the counter pharmaceutics and products, such as, deodorants; Tourette's Syndrome agents; tremor treatments; urinary tract agents, such as, acidifiers, alkalinizers; antispasmodics; benign prostatic hyperplasia treatment agents; calcium oxalate stone preventors; enuresis management agents; vaginal preparations, such as, antiinfectives, hormones and so on; vasodilators; vertigo treatment agents, Wilson's Disease treatments and so on.

Further examples of bioactive agent can include forms of drugs which may be modified, for example, as salts, ionized or hydrophilic forms that can be modified to remove such functional groups, modifications and the like to yield non-modified or other forms of bioactive agents which are poorly water soluble or water insoluble. If two or more bioactive agents are comprised in the pharmaceutical composition, at least one of the bioactive agents can be or has been modified to be water insoluble or poorly water soluble. Examples of such bioactive agents can include, analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate and the like); anesthetics (e.g., cyclopropane, enflurane, halothane, isoflurane, methoxyflurane, nitrous oxide, propofol and the like); antiasthmatics (e.g., azelastine, ketotifen, traxanox, amlexanox, cromolyn, ibudilast, montelukast, nedocromil, oxatomide, pranlukast, seratrodast, suplatast tosylate, tiaramide, zafirlukast, zileuton, beclomethasone, budesonide, dexamethasone, flunisolide, triamcinolone acetonide and the like); antibiotics (e.g., neomycin, streptomycin, chloramphenicol, cephalosporin, ampicillin, penicillin, tetracycline and the like); quinolone, fluoroquinolone, antidepressants (e.g., nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, trimipramine maleate, protriptyline hydrochloride and the like); antidiabetics (e.g., biguanides, hormones, sulfonylurea derivatives, and the like); antifungal agents (e.g., griseofulvin, ketoconazole, amphotericin B, nystatin, candicidin and the like); antihypertensive agents (e.g., propanolol, propafenone, oxyprenolol, nifedipine, reserpine, trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine and the like); anti-inflammatories (e.g., non-steroidal compounds, such as, indomethacin, naproxen, ibuprofen, ramifenazone, piroxicam and so on, and steroidal compounds, such as, cortisone, dexamethasone, fluazacort, hydrocortisone, prednisolone, prednisone and the like); antineoplastics (e.g., adriamycin, cyclophosphamide, actinomycin, bleomycin, daunorubicin, doxorubicin, epirubicin, gemcitabine, mitomycin, methotrexate, fluorouracil, carboplatin, carmustine (a R-chloro-nitrosourea (BCNU) compound), methyl-1-(-chloroethyl-cyclohexyl-1-nitrosourea (CCNU), cisplatin, etoposide, interferons, camptothecin and derivatives thereof, phenesterine, Taxol and derivatives thereof, taxotere and derivatives thereof, vinblastine, vincristine, tamoxifen, etoposide, piposulfan and the like); antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, dantrolene and the like); immunosuppressive agents (e.g., cyclosporine, azathioprine, mizoribine, FK506 (tacrolimus), rapamycin (sirolimus) and the like); antimigraine agents (e.g., ergotamine tartrate, propanolol hydrochloride, isometheptene mucate, dichloralphenazone and the like); sedatives/hypnotics (e.g., barbiturates (e.g., pentobarbital, pentobarbital sodium, secobarbital sodium and the like) or benzodiazapines (e.g., flurazepam hydrochloride, triazolam, tomazeparm, midazolam hydrochloride and the like); antianginal agents (e.g., beta-adrenergic blockers, calcium channel blockers (e.g., nifedipine, diltiazem hydrochloride and the like) and nitrates (e.g., nitroglycerin, isosorbide dinitrate, pentaerythritol tetranitrate, erythrityl tetranitrate and the like)); antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine and the like); antimanic agents (e.g., lithium carbonate); antiarrhythmics (e.g., bretylium tosylate, esmolol hydrochloride, verapamil hydrochloride, amiodarone, encamide hydrochloride, digoxin, digitoxin, mexiletine hydrochloride, disopyramide phosphate, procainamide hydrochloride, quinidine sulfate, quinidine gluconate, quinidine polygalacturonate, flecamide acetate, tocamide hydrochloride, lidocaine hydrochloride and the like); antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium and the like); antigout agents (e.g., colchicine, allopurinol and the like); anticoagulants (e.g., heparin, heparin sodium, warfarin sodium and the like); thrombolytic agents (e.g., urokinase, streptokinase, altoplase and the like); antifibrinolytic agents (e.g., aminocaproic acid); hemorheologic agents (e.g., pentoxifylline); antiplatelet agents (e.g., aspirin, empirin, ascriptin and the like); anticonvulsants (e.g., valproic acid, divalproate sodium, phenyloin, phenyloin sodium, clonazepam, primidone, phenobarbitol, phenobarbitol sodium, carbamazepine, amobarbital sodium, methsuximide, metharbital, mephobarbital, mephenyloin, phensuximide, paramethadione, ethotoin, phenacemide, secobarbitol sodium, clorazepate dipotassium, trimethadione and the like); antiparkinson agents (e.g., ethosuximide and the like); antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like); agents useful for calcium regulation (e.g., calcitonin, parathyroid hormone and the like); antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate and the like); antiviral agents (e.g., interferon γ, zidovudine, amantadine hydrochloride, ribavirin, acyclovir and the like); antimicrobials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium and the like), penicillins (e.g., ampicillin, amoxicillin, penicillin G benzathine, cyclacillin, ampicillin sodium, penicillin G K, penicillin V K, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, nafcillin sodium and the like), erythromycins (e.g., erythromycin ethylsuccinate, erythromycin, erythromycin estolate, erythromycin lactobionate, erythromycin stearate, erythromycin ethylsuccinate and the like), tetracyclines (e.g., tetracycline hydrochloride, doxycycline hyclate, minocycline hydrochloride and the like), and the like); anti-infectives (e.g., granulocyte-macrophage colony stimulating factor, GM-CSF); bronchodilators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate); anticholinergic agents (e.g., ipratropium bromide); xanthines (e.g., aminophylline, dyphylline, metaproterenol sulfate, aminophylline); mast cell stabilizers (e.g., cromolyn sodium); inhalant corticosteroids (e.g., flunisolide, beclomethasone dipropionate monohydrate and the like), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide and the like); hormones (e.g., androgens (e.g., danazol, testosterone cypionate, fluoxymesterone, ethyltostosterone, testosterone enanthate, methyltestosterone, fluoxymesterone, testosterone cypionate and the like); estrogens (e.g., estradiol, estropipate, conjugated estrogens and the like), progestins (e.g., methoxyprogesterone acetate, norethindrone acetate and the like), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate and the like), thyroid hormones (e.g., levothyroxine sodium); and the like); and the like; hypoglycemic agents (e.g., human insulin, purified beef insulin, purified pork insulin, glyburide, chlorpropamide, glipizide, tolbutamide, tolazamide and the like); hypolipidemic agents (e.g., clofibrate, dextrothyroxine sodium, probucol, lovastatin, niacin and the like); proteins (e.g., DNase, alginase, superoxide dismutase, lipase and the like); nucleic acids (e.g., sense or anti-sense nucleic acids encoding any therapeutically useful protein, including any of the proteins described herein and the like); agents useful for erythropoiesis (e.g., erythropoietin); antiulcer or antireflux agents (e.g., famotidine, cimetidine, ranitidine hydrochloride and the like); antinauseants or antiemetics (e.g., meclizine hydrochloride, nabilone, prochlorperazine, dimenhydrinate, promethazine hydrochloride, thiethylperazine, scopolamine and the like); oil-soluble vitamins (e.g., vitamins A, D, E, K and the like); mitotane, visadine, halonitrosoureas, anthrocyclines, ellipticine and the like; stimulator of interferon genes (STING) inhibitors, such as, C-176, C-170 and C-171; STING activators, such as, 3′,3′-cGAMP (3′,3′-cyclic GMP-AMP, Cyclic GMP-AMP, cGAMP); STING agonists, such as, SR-717 lithium, Alpha-mangostin or diABZI STING agonist (diABZI STING agonist-1, Compound 3), STING agonist-1 (G10), CF501 (Formula (1)), CF502 (Formula (5)), CF504 (Formula (7)), CF505 (Formula (8)), CF508 (Formula (4)), CF509 (Formula (6)), CF510 (Formula (2)), CF511 (Formula (9)) (Liu, et al., Cell Research, 1-19, 2022. https://doi.org/10.1038/s41422-022-00612-2), or MSA-2; STING antagonist such as, SN-011 (GUN35901) or H-151; indoleamine 2,3-dioxygenase (IDO or IDO-1) inhibitors or IDO1 inhibitors, such as, Epacadostat (INCB24360), BMS-986205, PF-0684003, Navoximod, Indoximod, NLG802 (Indoximod prodrug) or LY3381916; and a combination thereof.

In some cases, the bioactive agent can comprise any one of the bioactive agents listed above and hereafter. In some cases, the bioactive agent can comprise two or more of the bioactive agents listed above and hereafter.

In some cases, in any of the pharmaceutical composition disclosed herein, the bioactive agent can comprise a natural or synthetic small molecule-based drug, inorganic-based drug, biological drug, natural or synthetic large molecule-based drug, a derivative thereof, or a combination thereof.

In some cases, the bioactive agent can comprise immunoglobin, such as, IgG, IgM, one or more molecules disclosed and prepared according to processes and method described in U.S. Pat. No. 10,688,048, hereby incorporated by reference in entirety.

In some cases, the bioactive agent can comprise taxane, paclitaxel, docetaxel, cabazitaxel, larotaxel, milataxel, ortataxel, tesetaxel, Topoisomerase 1 (Top 1) inhibitor, camptothecin derivative, irinotecan (CPT-11), SN-38, topotecan, Topoisomerase 2 (Top 2) inhibitor, doxorubicin, etoposide, ciprofloxaxin, mammalian target of rapamycin (mTOR) inhibitor, at least one STING polypeptide or a part thereof, a nucleic acid encoding the STING polypeptide or a part thereof, a STING inhibitor, a STING activator, a STING agonist, a STING antagonist, a STING modulating molecule, an IDO inhibitor, an IDO1 inhibitor, or a combination thereof, wherein the mTOR inhibitor comprises everolimus, rapamycin, temsirolimus, zotarolimus, torin-1, torin-2, vistusertib, ridaforolimus, one or more dual phosphopinositide 3-kinase (PI3K)-mTOR inhibitors, one or more ATP-competitive mTORC1/2 inhibitors, a derivative thereof, or a combination thereof.

In some cases, the bioactive agent can comprise 7-ethyl-10-hydroxycamptothecin (SN-38). In some cases, bioactive agent can comprise 7-ethyl-10-hydroxycamptothecin (SN-38), irinotecan (also known as Camptosar, Campto, Onivyde, CPT-11), camptothecin (CPT), topotecan, or a combination thereof.

In some cases, the bioactive agent can comprise at least one STING polypeptide or a part thereof, a nucleic acid encoding the STING polypeptide or a part thereof, a STING inhibitor, a STING activator, a STING agonist, a STING antagonist, a STING modulating molecule, an IDO inhibitor, an IDO1 inhibitor, or a combination thereof. The bioactive agent can comprise RNA, mRNA, siRNA, single guide RNA (sgRNA), DNA, oligo, or a combination thereof, that each encodes one of the aforementioned STING polypeptide or a part thereof, a STING inhibitor, a STING activator, a STING agonist, a STING antagonist, a STING modulating molecule, or the IDO inhibitor or IDO1 inhibitor.

In some cases, the bioactive agent can comprise STING (stimulator of interferon genes) protein, STING agonists, STING activators, STING inhibitors, STING antagonists or a combination thereof. In some cases, the bioactive agent can comprise one or more IDO or IDO1 inhibitors. Any of the STING protein, STING agonists, STING activators, STING inhibitors, STING antagonists, IDO inhibitors or IDO1 inhibitors, disclosed herein or discovered thereafter can be suitable. In some cases, the bioactive agent can comprise STING modulating molecules, such as, benzimidazole compounds disclosed by Liu, et al. (Cell Research, 1-19, 2022), in patent publications WO2017175156A1 and WO2020156363, pyridinyl imidazole compounds disclosed in patent publications WO2019134705, WO2020010451 and US20200031825, or a combination.

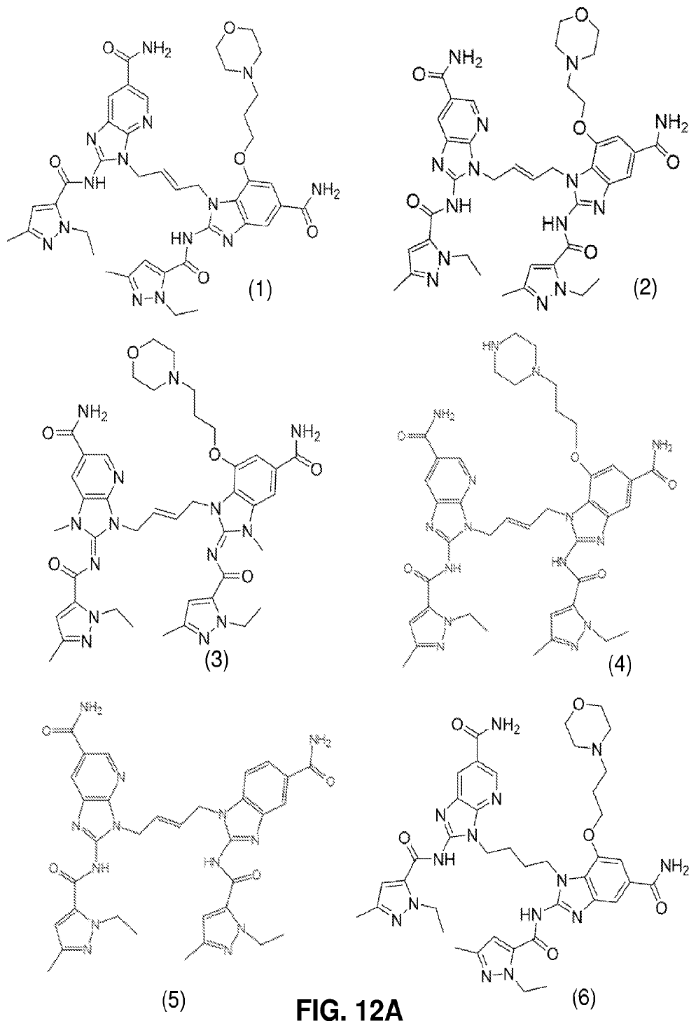

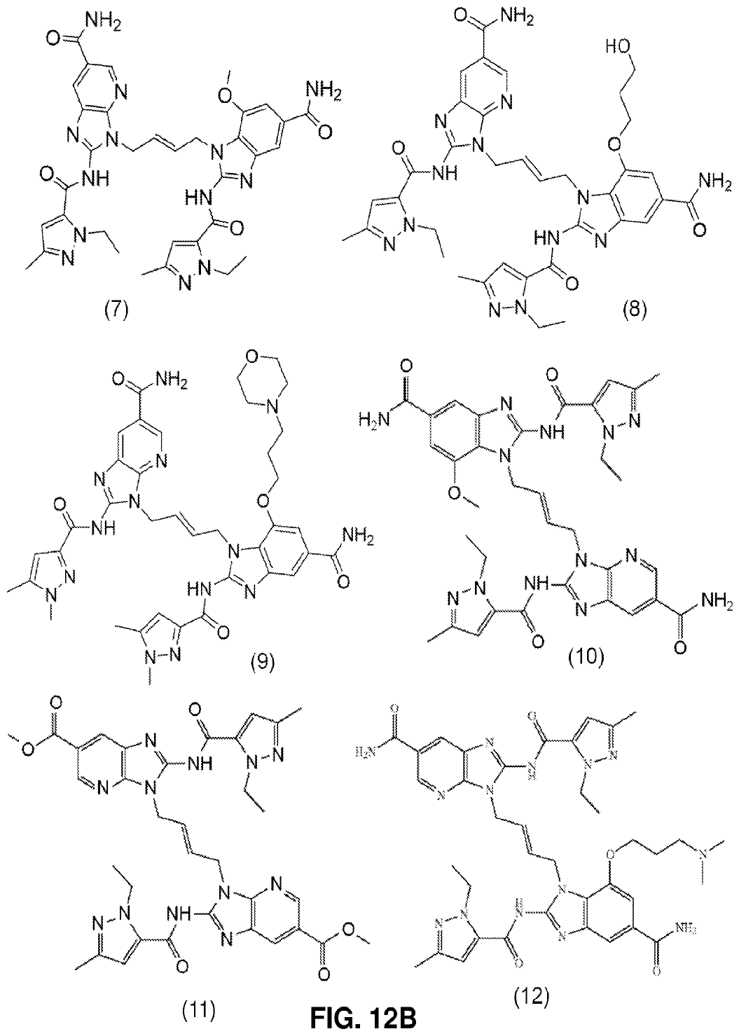

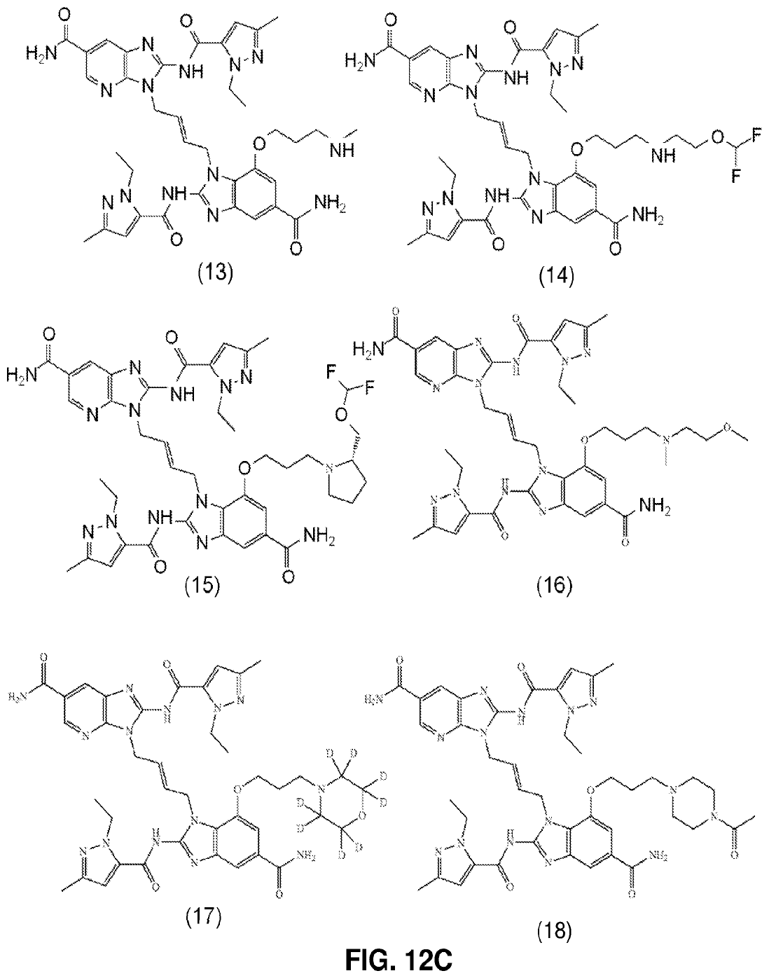

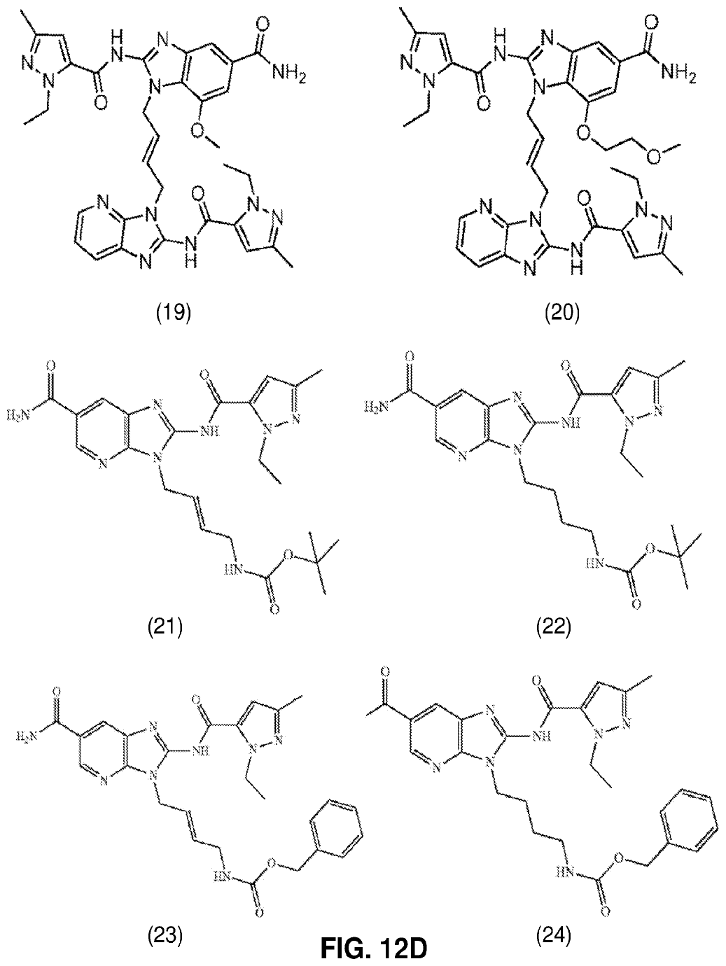

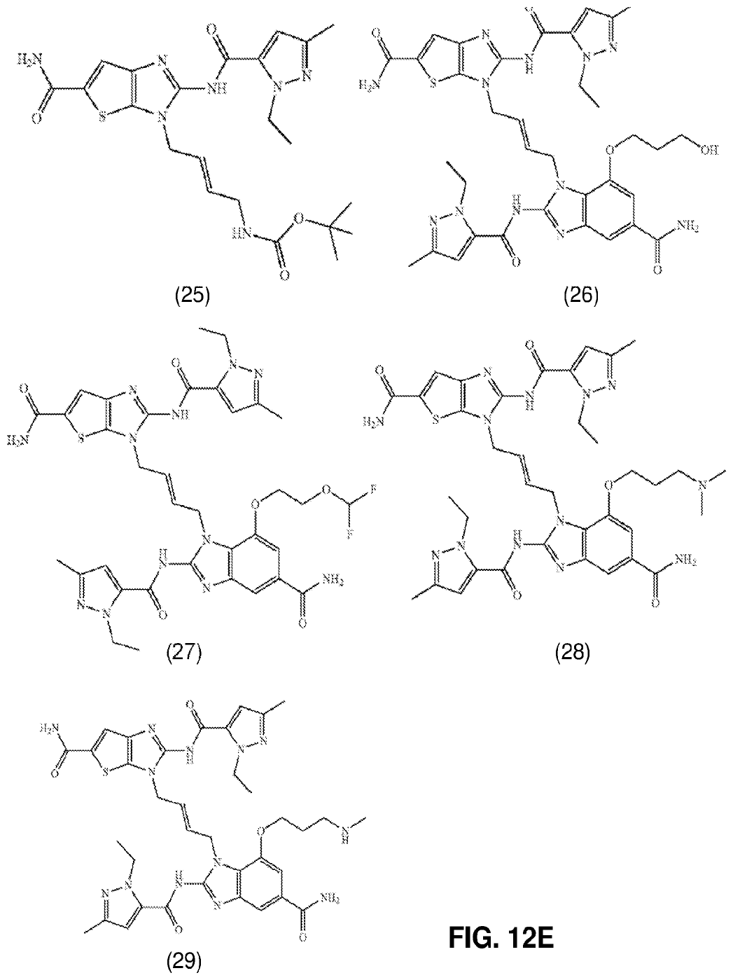

In some cases, the bioactive agent can comprise one or more STING agonists. In some cases, the STING agonist can comprise one or more compounds having the formula (1)-(29) (FIG. 12A-FIG. 12E),

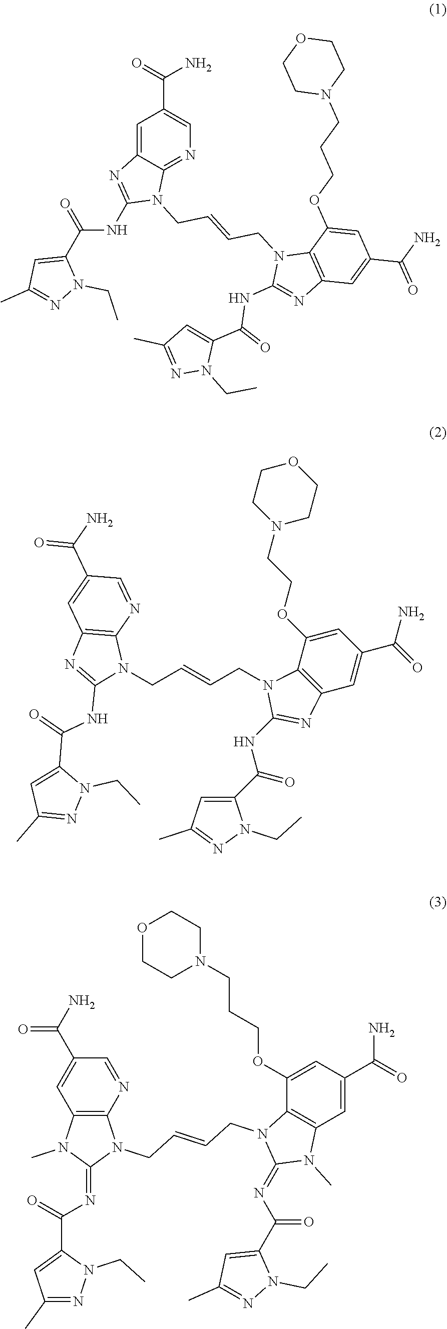 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCOCC3)c21 ; CCn1nc(C)cc1C(=O)/N=c1\n(C)c2cc(C(N)=O)cnc2n1C/C=C/Cn1/c(=N/C(=O)c2cc(C)nn2CC)n(C)c2cc(C(N)=O)cc(OCCCN3CCOCC3)c21]

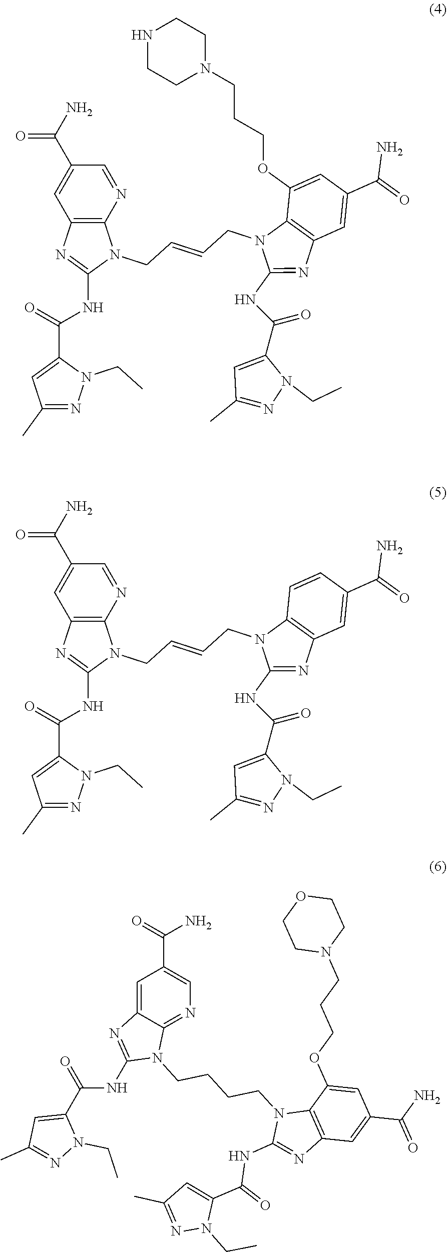 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cnc21 ; CCn1nc(C)cc1C(=O)Nc1nc2cc(C(N)=O)cnc2n1CCCCn1c(NC(=O)c2cc(C)nn2CC)nc2cc(C(N)=O)cc(OCCCN3CCOCC3)c21]

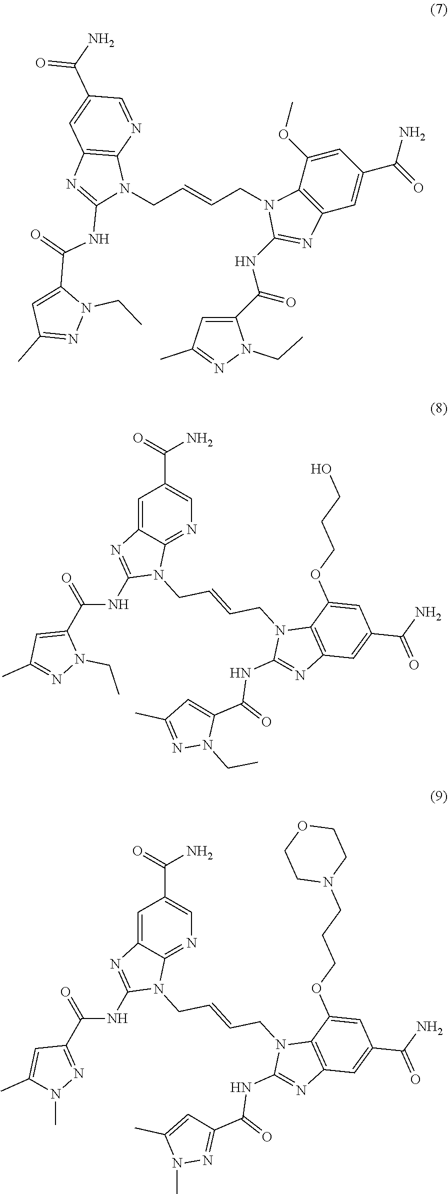 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OC)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO)c21 ; Cc1cc(C(=O)Nc2nc3cc(C(N)=O)cnc3n2C/C=C/Cn2c(NC(=O)c3cc(C)n(C)n3)nc3cc(C(N)=O)cc(OCCCN4CCOCC4)c32)nn1C]

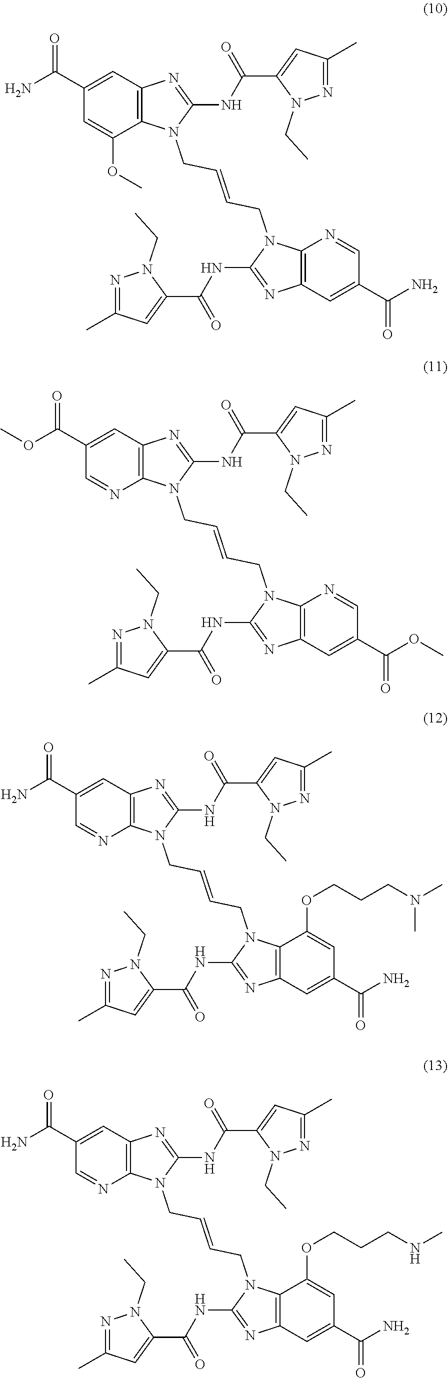

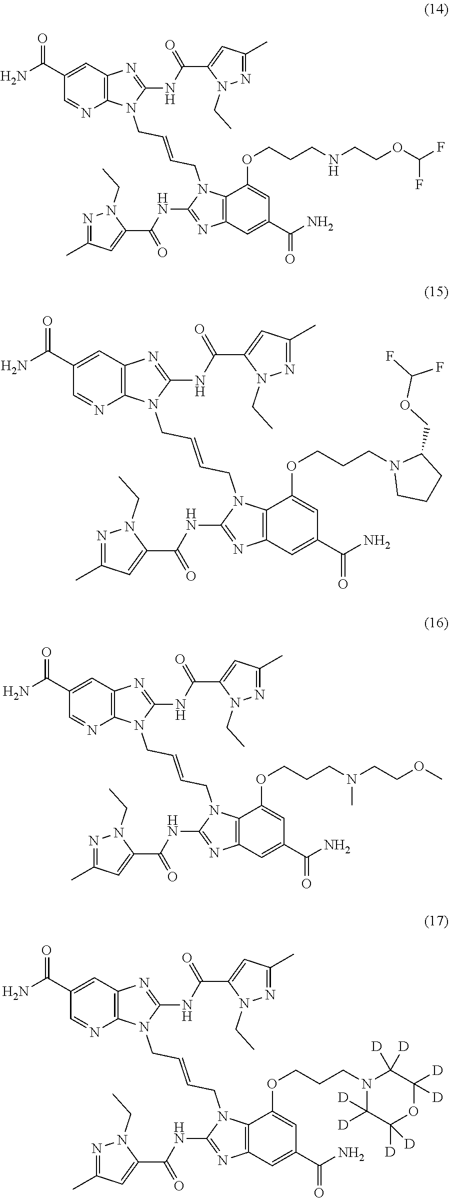 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNCCOC(F)F)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC[C@H]3COC(F)F)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN(C)CCOC)c21 ; [2H]C1([2H])OC([2H])([2H])C([2H])([2H])N(CCCOc2cc(C(N)=O)cc3nc(NC(=O)c4cc(C)nn4CC)n(C/C=C/Cn4c(NC(=O)c5cc(C)nn5CC)nc5cc(C(N)=O)cnc54)c23)C1([2H])[2H]]

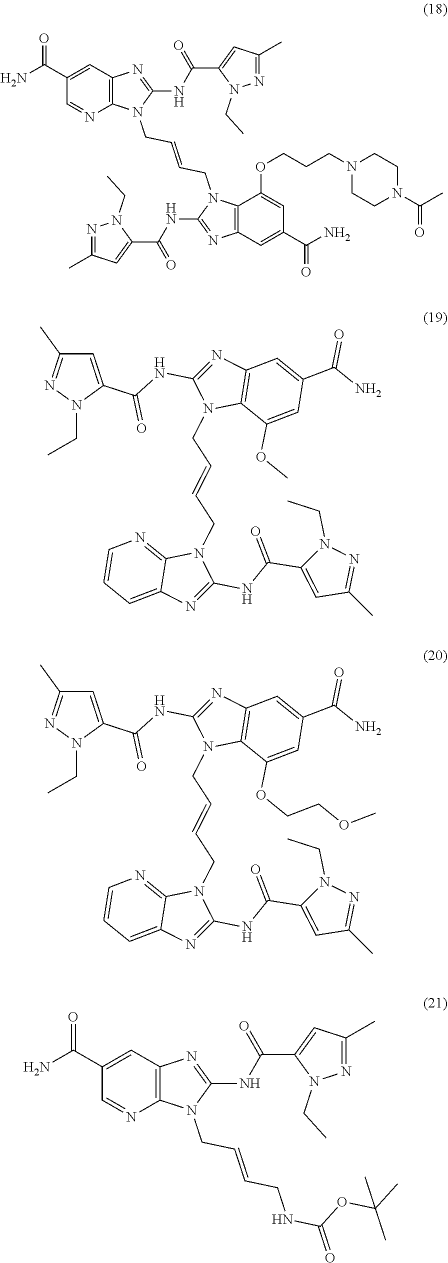

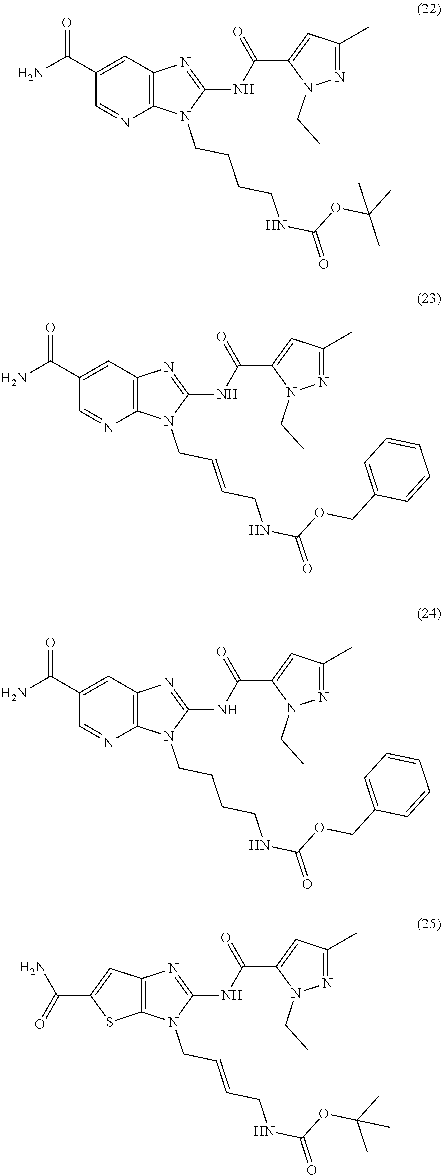 [SMILES: CCn1nc(C)cc1C(=O)Nc1nc2cc(C(N)=O)cnc2n1CCCCNC(=O)OC(C)(C)C ; CCn1nc(C)cc1C(=O)Nc1nc2cc(C(N)=O)cnc2n1C/C=C/CNC(=O)OCc1ccccc1 ; CCn1nc(C)cc1C(=O)Nc1nc2cc(C(N)=O)cnc2n1CCCCNC(=O)OCc1ccccc1 ; CCn1nc(C)cc1C(=O)Nc1nc2cc(C(N)=O)sc2n1C/C=C/CNC(=O)OC(C)(C)C]

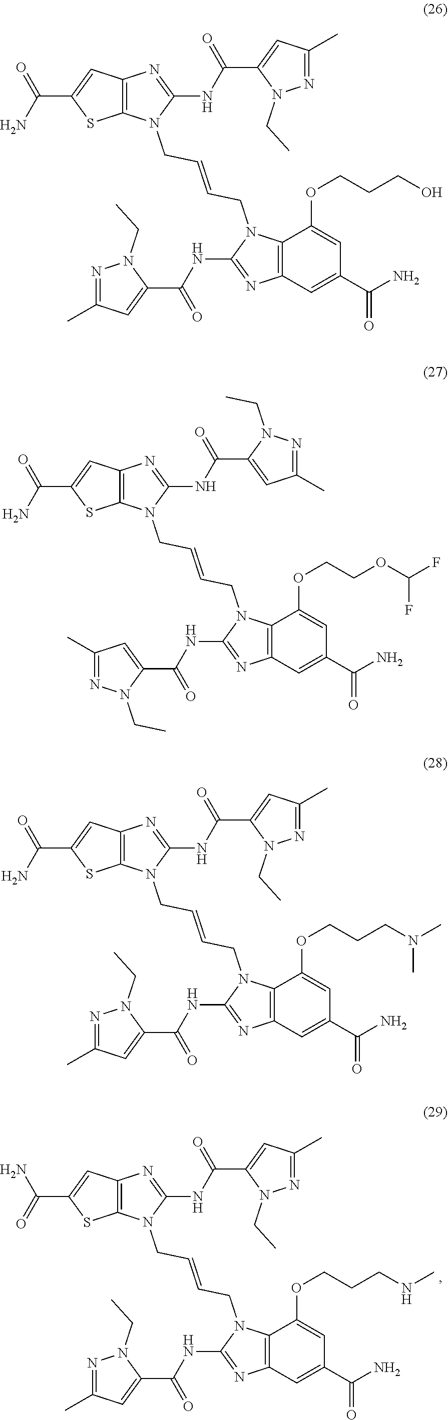 [SMILES: CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCO)c21 ; CCn1nc(C)cc1C(=O)Nc1nc2cc(C(N)=O)sc2n1C/C=C/Cn1c(NC(=O)c2cc(C)nn2CC)nc2cc(C(N)=O)cc(OCCOC(F)F)c21 ; CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCN(C)C)c21 ; CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCNC)c21]

corresponding salt, solvate, prodrug, isomer thereof, or a combination thereof.

In some cases, the bioactive agent can comprise a compound having

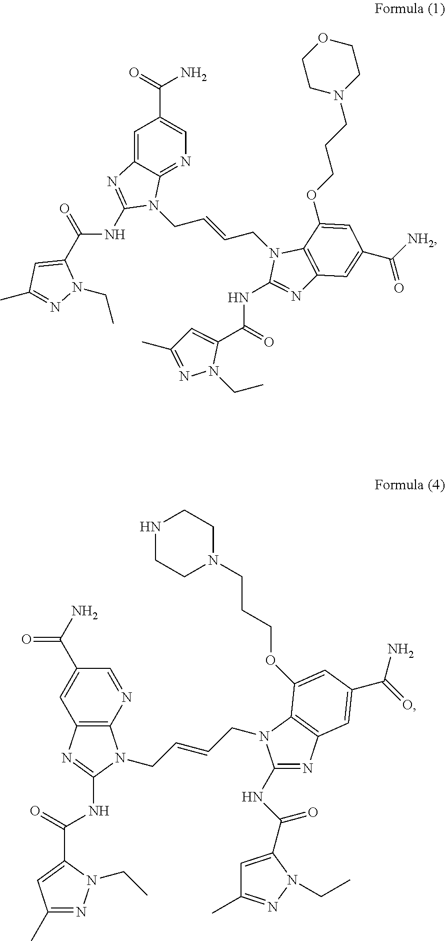 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

a pharmaceutically acceptable salt thereof, solvate thereof, prodrug thereof, isomer thereof, or a combination thereof.

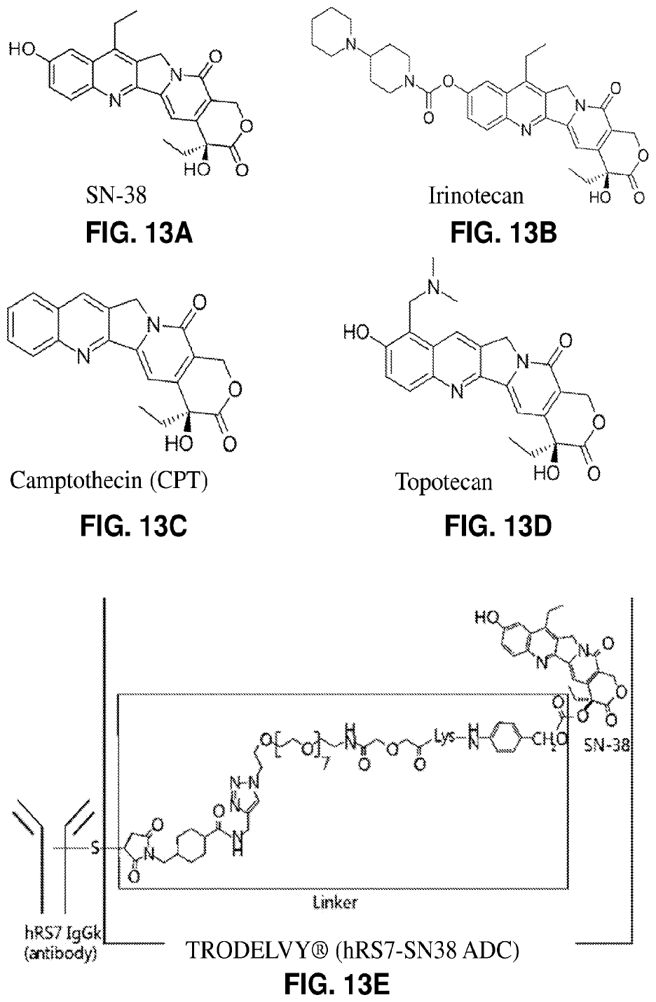

In some cases, the bioactive agent can comprise Topoisomerase 1 (Top 1) inhibitor, camptothecin derivative, irinotecan (CPT-11), SN-38, topotecan, Topoisomerase 2 (Top 2) inhibitor, doxorubicin, etoposide, ciprofloxaxin, or a combination thereof. SN-38, with chemical structure 7-ethyl-10-hydroxycamptothecin (FIG. 13A), is a Topoisomerase 1 (Top 1) inhibitor. Throughout this disclosure, the terms “7-ethyl-10-hydroxycamptothecin” and “SN-38” may be used interchangeably. Irinotecan (FIG. 13B), also known as Camptosar®, Campto, Onivyde®, under respective trademarks, CPT-11 ((S)-(+)-7-Ethyl-10-hydroxycamptothecine 10-[1,4′-bipiperidine]-1′-carboxylate, monohydrochloride), is a water soluble camptothecin analog and a prodrug of SN-38. Irinotecan can be converted to SN-38 in vivo via metabolism (Chabot GG. Clinical pharmacokinetics of irinotecan, Clin. Pharmocokinet. 1997, 33 (4), 245-259). Camptothecin (CPT) (FIG. 13C) is a pentacyclic monoterpene alkaloid naturally occurring in the tree bark and stem of Camptotheca acuminiata. Topotecan, also known as Hycamtin (FIG. 13D) is also a water soluble camptothecin derivative that is approved in both IV injectable and oral forms. Examples of SN-38 antibody drug complex (ADC) (SN-38 ADC) can include TRODELVY® (hRS7-SN38 ADC) (FIG. 13E) under respective trademark.

In some cases, the nanoaggregate can be water soluble and can comprise a polymer and at least one bioactive agent that is water insoluble or poorly water soluble. The pharmaceutical composition comprising the nanoaggregate can be soluble in an aqueous solution to produce at least 1 mg/mL of the bioactive agent in the aqueous solution. In some cases, the pharmaceutical composition comprising the nanoaggregate can be soluble in an aqueous solution to produce at least 2 mg/mL of the bioactive agent disclosed herein, or a combination thereof, in the aqueous solution. In some cases, the pharmaceutical composition comprising the nanoaggregate can be soluble in an aqueous solution to produce at least 1 mg/mL, 1.5 mg/mL, 2 mg/mL, 2.5 mg/mL, 3 mg/mL, 3.5 mg/mL, 4 mg/mL, 4.5 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, or more, of the bioactive agent disclosed herein, or a combination thereof, in the aqueous solution.

Any polymer disclosed here can be suitable. In some examples, the polymer can comprise a polyoxazoline (POX) that comprises a linear portion, a branched portion, or a combination thereof, and wherein the polyoxazoline (POX) comprises poly(2-oxazoline), poly(2-methyloxazoline), poly(2-ethyloxazoline), poly(2-propyloxazoline), poly(isopropyloxazoline) (PiPOX), or a combination thereof. In some examples, the polyoxazoline can be poly(2-ethyloxazoline).

As used throughout this disclosure, the nanoaggregate can be of a size less than 150 nm before lyophilization. In some cases, the nanoaggregate can be of a size less than 120 nm before lyophilization. By “less than”, it means the size can be less than the defined size in nm and can be about 0 nm, i.e., a solution of the nanoaggregate can be a clear solution with no measurable particles or aggregates. For example, “less than 150 nm” means in a range of from 0 nm to 150 nm and “less than 120 nm” means in a range of from 0 nm to 120 nm. In some cases, the size of the nanoaggregates or nanoparticles, can range from about 0.01 nm to about 100 nm before lyophilization. In some cases, the size of the nanoaggregates or nanoparticles, can range from about 0.01 nm to about 120 nm before lyophilization. In some cases, the size of the nanoaggregates or nanoparticles, can range from about 0.01 nm to about 150 nm before lyophilization. In some cases, the size of the nanoaggregates or nanoparticles, can range from about 50 to about 100 nm before lyophilization. In some cases, the size of the nanoaggregates or nanoparticles, can range from about 50 to about 120 nm before lyophilization. Particle size can be measured by light scattering.

In some cases, the pharmaceutical composition of this disclosure, wherein the nanoaggregate can further comprise a targeting moiety comprises an antibody, an antigen-binding portion thereof, an antigen, a cell receptor, a cell receptor ligand, a ligand for a cellular protein, a ligand for a membrane protein, a small molecule ligand, a lectin ligand, or a combination thereof.

In some cases, the nanoaggregate can be free from human serum albumin, organic solvent, detergent, or oil. In some cases, the nanoaggregate can be free from human serum albumin, organic solvent, detergent, oil or free acid. In some cases, the nanoaggregate can be free from human serum albumin. In some cases, the nanoaggregate can be free from organic solvent. In some cases, the nanoaggregate can be free from detergent. In some cases, the nanoaggregate can be free from oil. In some cases, the nanoaggregate can be free from free acid. In some cases, the nanoaggregate can be free from materials selected from the group consisting of human serum albumin, organic solvent, detergent, oil, free acid, and a combination thereof.

In some cases, the pharmaceutical composition can be free from human serum albumin, organic solvent, detergent, or oil. In some cases, the pharmaceutical composition can be free from human serum albumin, organic solvent, detergent, oil or free acid. In some cases, the pharmaceutical composition can be free from human serum albumin. In some cases, the pharmaceutical composition can be free from organic solvent. In some cases, the pharmaceutical composition can be free from detergent. In some cases, the pharmaceutical composition can be free from oil. In some cases, the pharmaceutical composition can be free from free acid. In some cases, the pharmaceutical composition can be free from materials selected from the group consisting of human serum albumin, organic solvent, detergent, oil, free acid, and a combination thereof.

In some cases, the bioactive agent can comprise one or more STING agonists. In some cases, the bioactive agent can comprise at least a compound having Formula (1)-Formula (29), a pharmaceutically acceptable salt thereof, solvate thereof, prodrug thereof, isomer thereof, or a combination thereof (FIG. 12A-FIG. 12E).

In some cases, the bioactive agent can comprise a compound having

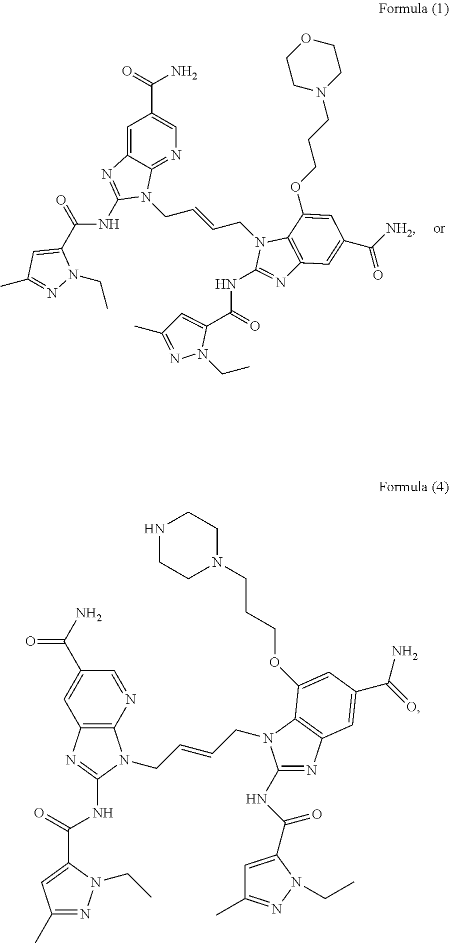 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

a pharmaceutically acceptable salt thereof, solvate thereof, prodrug thereof, isomer thereof, or a combination thereof.

The pharmaceutical composition of this disclosure can be a drug for treating or preventing a disease selected from one or more immune disorders, infectious diseases, cancers, and a combination thereof. Immune disorders can include immunodeficiency disorders, overactive immune disorders, autoimmune diseases, and other disorders or symptoms that have abnormal immune systems. In some cases, the immune disorders can be various autoinflammatory, autoimmune and degenerative diseases, such as, those associated with STING (stimulator of interferon genes) signaling pathway or IDO pathway. In some cases, immune disorders can be associated with STING mediated inflammation in infection, cellular stress and tissue damage. In some cases, the pharmaceutical composition of this disclosure can be a drug for cancer immunotherapy based on immune checkpoint blockade. In some cases, the pharmaceutical composition of this disclosure can comprise STING protein, STING agonists, STING activators, STING inhibitors, STING antagonists, IDO inhibitors, IDO1 inhibitors, or a combination thereof. In some cases, the pharmaceutical composition of this disclosure can comprise one or more STING inhibitors. In some cases, the pharmaceutical composition of this disclosure can comprise one or more STING activators.

The term “cancer” or “cancers” used herein and throughout this disclosure refers to cancer or tumor and can include malignant tumors and benign tumors such as, solid tumors and cancers of the blood, such as, leukemia's. Malignant tumors can spread into, or invade, nearby tissues. In addition, as these tumors grow, some cancer cells can break off and travel to distant places in the body through the blood or the lymph system and form new tumors (metastatic tumor) far from the original tumor (primary cancer). A cancer can include a primary cancer or a metastatic tumor. The pharmaceutical composition disclosed herein can be a cancer treatment drug for treating one or more cancers. In some cases, the term “cancer” or “cancers” used herein can include one or more cancer selected from acoustic neuroma, Acute Lymphoblastic Leukemia (adult), Acute Lymphoblastic Leukemia (pediatric), Acute Myeloid Leukemia, adenocarcinoma, Anal Cancer, Anemia and Neutropenia (Low Red and White Blood Cell Counts), basal cell carcinoma, Basal Cell Skin Cancer, B-Cell Lymphomas (Diffuse Large B-Cell Lymphoma), B-Cell Lymphomas (Follicular Lymphoma), B-Cell Lymphomas (Mantle Cell Lymphoma), bile duct carcinoma, biliary track cancer, Bladder Cancer, bladder carcinoma, Bone Cancer, Brain Cancer (Gliomas), brainstem glioma, breast cancer, Breast Cancer (DCIS Breast Cancer), Breast Cancer (Invasive Breast Cancer), Breast Cancer (Metastatic Breast Cancer), triple-negative breast cancer, estrogen receptor (ER)(+) locally advanced or metastatic breast cancer, bronchogenic carcinoma, Central Nervous System (CNS) Cancers (Primary Central Nervous System Lymphoma), cervical cancer, choriocarcinoma, Chronic Lymphocytic Leukemia, chronic lymphocytic lymphoma, Chronic Myeloid Leukemia, colon cancer, colon carcinoma, Colorectal Cancer (CRC), diffuse large B cell lymphoma, ependymoma, Esophageal Cancer, Gallbladder and Bile Duct Cancers, gastric cancers, germinoma, glioblastoma astrocytoma, mixed gliomas, Graft-Versus-Host Disease, head and neck cancers, Head and Neck Cancers (Nasopharyngeal Cancer), Head and Neck Cancers (Oral Cancers), Head and Neck Cancers (Oropharyngeal Cancer), hemangioblastoma, hepatocellular carcinoma, hepatoma, Hodgkin Lymphoma, Kidney Cancer, leukemias, Liver Cancer, Lung Cancer, Lung Cancer (Non-Small Cell Lung Cancer (NSCLC)—Early and Locally Advanced), Lung Cancer (Non-Small Cell Lung Cancer—Metastatic), Lung Cancer NSCLC (Non-Small Cell Lung Cancer), Lung Cancer (Small Cell Lung Cancer), lymphoid malignancy, Malignant Pleural Mesothelioma, medullary carcinoma, medullary thyroid carcinoma, medulloblastoma, melanoma, menangioma, Multiple Myeloma, Mycosis Fungoides/Sezary Syndrome, Myelodysplastic Syndromes, Myeloproliferative Neoplasms, neuroblastoma, neuroendocine tumor (advanced), Neuroendocrine Tumors, oligodendroglioma, ovarian cancer, pancreatic cancer, papillary adenocarcinomas, papillary carcinoma, papillary thyroid carcinoma, Peripheral T-Cell Lymphoma, perivascular epithelioid cell (PEC) tumor (PEComa), perivascular epithelioid cell tumor (advanced unresectable or metastatic malignant), pheochromocytomas, pinealoma, Primary Cutaneous Lymphomas, prostate cancer, Prostate Cancer (Advanced Stage), Prostate Cancer (Early Stage), Rectal Cancer, recurrent endometrial cancer, relapsed ER(+) high-grade ovarian cancer, relapsed or refractory non-Hodgkin lymphoma, renal cancer (metastatic clear cell), renal cell carcinoma, retinoblastoma and brain metastases, Schwannoma craniopharyogioma, sebaceous gland carcinoma, seminoma, soft tissue sarcoma, squamous cell carcinoma, Squamous Cell Skin Cancer, Stomach Cancer, sweat gland carcinoma, Systemic Mastocytosis, testicular tumor, thyroid cancer, Uterine Cancer, uterine sarcoma, Waldenstrom Macroglobulinemia, and a combination thereof.

In some cases, the one or more cancers can be selected from any of the cancers described above. In some cases, the one or more cancers can be selected from thyroid cancer, relapsed ER(+) high-grade ovarian cancer, ER(+) locally advanced or metastatic breast cancer, advanced neuroendocine tumor, diffuse large B cell lymphoma, advanced solid tumors, metastatic clear cell renal cancer, relapsed or refractory non-Hodgkin lymphoma, chronic lymphocytic lymphoma, recurrent endometrial cancer, perivascular epithelioid cell tumor (PEComa), advanced unresectable or metastatic malignant perivascular epithelioid cell tumor, ovarian cancers, lung cancers, NSCLC (Non-Small Cell Lung Cancer), small cell lung cancer, biliary track cancer, bladder cancer, cervical cancer, soft tissue sarcoma, uterine sarcoma, colon cancers, gastric cancers, melanoma, head and neck cancers, pancreatic cancers, one or more metastatic cancers derived therefrom, and a combination thereof. In some cases, the one or more cancers can be advanced unresectable or metastatic malignant perivascular epithelioid cell tumor (PEComa).

The pharmaceutical composition of this disclosure can be a drug for treating or preventing one or more of the diseases disclosed herein.

In some cases, the pharmaceutical composition can comprise two or more bioactive agents, wherein at least one of the two or more bioactive agents is water insoluble or poorly water soluble. In some cases, at least one of the two or more bioactive agents is paclitaxel. In some cases, at least one of the two or more bioactive agents is rapamycin. In some cases, the pharmaceutical composition can comprise paclitaxel and one or more additional bioactive agents disclosed herein that is different from paclitaxel. In some cases, the pharmaceutical composition can comprise rapamycin and one or more additional bioactive agents selected from gemcitabine, paclitaxel, docetaxel, cabazitaxel, larotaxel, milataxel, ortataxel, tesetaxel, temozolomide, platinum-based antineoplastic agent, daunorubicin, doxorubicin, epirubicin, mitomycin, methotrexate, fluorouracil, carboplatin, carmustine (bis-chloroethylnitrosourea, also known as BCNU or BiCNU), methyl-CCNU, cisplatin, vinorelbine, capecitabine or a combination thereof. In some cases, the pharmaceutical composition can comprise two or more bioactive agents in the nanoaggregate. In some cases, the pharmaceutical composition can comprise at least one water insoluble or poorly water soluble bioactive agent, such as, rapamycin, in nanoaggregate and one or more additional bioactive agents that are either included in the nanoaggregate or not included in the nanoaggregate. The pharmaceutical composition can comprise a nanoaggregate comprises a polymer and two or more bioactive agents that each is water insoluble or poorly water soluble. The pharmaceutical composition can comprise a nanoaggregate comprises a polymer and at least one bioactive agent that is water insoluble or poorly water soluble, and one or more additional bioactive agents that each is water soluble. The pharmaceutical composition can comprise a nanoaggregate comprising rapamycin, a taxane and a gemcitabine in one example, a nanoaggregate comprising rapamycin and a taxane in another example and a nanoaggregate comprising a rapamycin and a temozolomide in yet another example.

The term “a combination thereof” used for a combination of the bioactive agents disclosed above means a combination of two or more bioactive agents, wherein such combination does not have undesired effect, such as, an undesired interaction between or among the bioactive agents. It is understood that some of combinations of the bioactive agents may not be suitable, or may not be desirable, such as, those having undesired interactions. For example, a combination of theophylline and ciprofloxacin or warfarin and diflunisal may not be suitable. These combinations or any combinations determined by appropriate guidelines or regulations as not suitable are thus excluded.

The pharmaceutical compositions of this invention disclosed herein can comprise rapamycin in a range of from 1 mg/ml to 10 mg/ml in an aqueous solution that is free from human serum albumin, organic solvent, detergent, or oil. The pharmaceutical compositions of this invention disclosed herein can comprise rapamycin in a range of from 1 mg/ml to 10 mg/ml in an aqueous solution that is free from human serum albumin, organic solvent, detergent, oil or free acid. Rapamycin itself is insoluble in water. A pharmaceutical composition of this invention can comprise rapamycin in a range of from 1 mg/ml to 10 mg/ml in one example, 2 mg/ml to 10 mg/ml in one example, 2 mg/ml to 7 mg/ml in another example, 2 mg/ml to 6 mg/ml in yet another example, 3 mg/ml to 10 mg/ml in yet another example, 3 mg/ml to 6 mg/ml in yet another example or 3 mg/ml to 5 mg/ml in yet another example, in an aqueous solution that is free from human serum albumin, organic solvent, detergent, oil or free acid.

As mentioned above, the nanoaggregate can be of a size less than 150 nm before lyophilization. In some cases, the nanoaggregate can be of a size less than 120 nm before lyophilization. In some cases, the nanoaggregate can be of a size in a range of from about 0.01 nm or about 0 nm to about 150 nm before lyophilization. The nanoaggregate can be of a size in a range of from about 50 nm to about 150 nm before lyophilization. In some cases, the size of the nanoaggregates or nanoparticles, can range from about 50 to about 100 nm before lyophilization. In some cases, the size of the nanoaggregates or nanoparticles, can range from about 50 to about 120 nm before lyophilization. In a further example, the nanoaggregate can be of a size in a range of from about from 70 to 90 nm before lyophilization. Particle size can be measured by light scattering.

In any of the pharmaceutical compositions disclosed above and hereafter, the nanoaggregate can have a weight ratio of polymer to bioactive agent in a range of from about 2:1 to about 200:1. The nanoaggregate can have a weight ratio of the polymer to the bioactive agent in a range of from about 2:1 to about 200:1 in one example, about 2:1 to about 150:1 in another example, about 2:1 to about 120:1 in yet another example, about 2:1 to about 100:1 in yet another example, about 2:1 to about 80:1 in yet another example, about 2:1 to about 60:1 in yet another example, about 2:1 to about 40:1 in yet another example, about 2:1 to about 30:1 in yet another example, about 2:1 to about 20:1 in yet another example, about 2:1 to about 15:1 in another example, about 2:1 to about 10:1 in yet another example, about 2:1 to about 8:1 in yet another example, about 5:1 to about 10:1 in yet another example, about 5:1 to about 8:1 in yet another example, 5:1 in yet a further example, about 6:1 to about 8:1 in yet another example, 6:1 in yet a further example, 7:1 in yet a further example, 7.5:1 in yet a further example, and 8:1 in yet a further example. When pharmaceutical composition comprises two or more bioactive agents, the ratio of polymer to bioactive agent can be based on the total weight of polymer and the bioactive agents.

In some cases, the pharmaceutical composition of interest can be an adjuvant for a vaccine.

In some cases, the pharmaceutical composition can be a prophylactic vaccine, a therapeutic vaccine, or a combination thereof, wherein the pharmaceutical composition can further comprise at least one immune agent for stimulating an immune response in a subject in need thereof. The immune agent can comprise inactive microbe selected from bacteria, viruses, fungi, protozoa, worms, parasites, prions, a part thereof, or a combination thereof; toxins; nucleic acids encoding the toxins; proteins; nucleic acids encoding the proteins; oligo nucleic acids; DNA's; RNA's; mRNA's; siRNA's; sgRNA's; fragments thereof; or a combination thereof.

In some cases, the pharmaceutical composition can be formulated for treating or preventing at least one infectious disease. In some cases, the pharmaceutical composition can be formulated for treating or preventing at least one infectious disease selected from Chickenpox (Varicella), Coronaviruses, Dengue, Diphtheria, Ebola, Flu (Influenza), Hepatitis, Hib Disease, HIV/AIDS, Human Papillomavirus (HPV), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, and a combination thereof.

In some cases, the pharmaceutical composition can have a pH value in a range of from 3.0 to 6.9. In some cases, the pharmaceutical composition can have a pH value in a range of from 4.0 to about 6.9, or 5.6 to about 6.9. The pH value can be measured in an aqueous solution of the pharmaceutical composition.

In some cases, the pharmaceutical composition can comprise one more of the bioactive agents disclosed herein, a derivative thereof, or a combination thereof, wherein in a range of from 1% to 100% of the second terminal group is free from primary amine, and wherein in a range of from 1% to 100% of the second terminal group comprises hydroxyl group.

In some cases, the pharmaceutical composition can comprise the mTOR inhibitor that comprises everolimus, rapamycin, temsirolimus, zotarolimus, torin-1, torin-2, vistusertib, ridaforolimus, a derivative thereof, or a combination thereof, wherein in a range of from 1% to 100% of the second terminal group is free from primary amine, and wherein in a range of from 1% to 100% of the second terminal group comprises hydroxyl group.

In some cases, the pharmaceutical composition can comprise the mTOR inhibitor that comprises everolimus, rapamycin, temsirolimus, zotarolimus, torin-1, torin-2, vistusertib, ridaforolimus, a derivative thereof, or a combination thereof, wherein in a range of from 50% to 100% of the second terminal group is free from primary amine, and wherein in a range of from 50% to 100% of the second terminal group comprises hydroxyl group.

In some cases, the pharmaceutical composition can comprise the mTOR inhibitor that comprises everolimus, rapamycin, temsirolimus, zotarolimus, torin-1, torin-2, vistusertib, ridaforolimus, a derivative thereof, or a combination thereof, wherein in a range of from 80% to 100% of the second terminal group is free from primary amine, and wherein in a range of from 80% to 100% of the second terminal group comprises hydroxyl group.

In some cases, the pharmaceutical composition can comprise the mTOR inhibitor that comprises everolimus, rapamycin, temsirolimus, zotarolimus, torin-1, torin-2, vistusertib, ridaforolimus, a derivative thereof, or a combination thereof, wherein in a range of from 90% to 100% of the second terminal group is free from primary amine, and wherein in a range of from 90% to 100% of the second terminal group comprises hydroxyl group.

In some cases, the pharmaceutical composition can further comprise one or more subsequent bioactive agents selected from a protein, a peptide, an antibody, a fragment of an antibody, a chemical compound, a small molecule drug, one or more chemotherapy drugs, and a combination thereof.

The pharmaceutical composition disclosed above and hereafter can further comprise an additional bioactive agent that is formulated free from the polymer, specifically the polymer disclosed herein. The phrase “additional bioactive agent that is formulated free from the polymer” refers to a bioactive agent formulation that comprises a bioactive agent and is free from the polymer disclosed herein, wherein the additional bioactive agent can be a salt, a base, a bioactive agent formulated with organic solvent, detergent, oil or free acid, protein, lipid or a combination thereof. In one particular example, the additional taxane is free from the polyoxazoline (POX) polymer disclosed herein. In some cases, the pharmaceutical composition can comprise an additional taxane formulated with human serum albumin in one example, taxane formulated with ethanol or Cremophor® (polyethyoxylated castor oil) in another example, taxane modified with acid, ammonium, alkyls, or aryls in yet another example, taxane formulated in a lipid in yet another example, taxane formulated in a cationic lipid in yet another example, and a combination thereof in a further example. Commercially available taxane formulations, such as, Abraxane© available from Celgene under respective trademark and Taxol® available from Bristol-Myers Squibb under respective trademark can be suitable. The pharmaceutical composition can further comprise Abraxane in one embodiment, Taxol in another embodiment, and a combination of Abraxane and Taxol in yet another embodiment.

The term “soluble in an aqueous solution” refers to a solution that comprises no detectable particles or has particles that can be filtered through a 0.22 μm filter with a filtration rating (R_{f}) through the 0.22 μm filter in a range of from 50 to 100 percent. As used throughout this disclosure, the term “0.22 μm filter” refers to a filter assembly having a 0.22 μm filtration pore size. The term “0.8 μm filter” refers to a filter assembly having a 0.8 μm filtration pore size.

In any of the pharmaceutical compositions disclosed above and hereafter, the nanoaggregate can have a filtration rating through a 0.22 μm filter in a range of from 50 to 100 percent. The filtration rating can be expressed as R_{f} and is defined in detail in latter sections of this disclosure.

In embodiments, the nanoaggregate can have a filtration rating at least 50%, at least 60%, at least 70%, at least 80%, at least 90% or more; or in a range of 50-100%, 55-100%, 60-100%, 65-100%, 70-100%, 75-100%, 80-100%, 85-100%, 90-100%, 95-100%, 55-95%, 65-95%, 75-95%, 85-95%, 50-95%, 60-95%, 70-95%, 80-95%, 90-95%, 50-90%, 60-90%, 70-90%, 80-90%, 55-90%, 65-90%, 75-90%, 85-90%; or, in any rating, must be 50% or greater.

The nanoaggregate can be filtered through a 0.22 μm filter to produce a sterilized nanoaggregate prior to lyophilizing, wherein the nanoaggregate can have a filtration rating (R_{f}) through a 0.22 μm filter in a range of from 50 to 100 percent. In another example, the pharmaceutical composition can be filtered through a 0.22 μm filter to produce a filtered pharmaceutical composition.

Briefly, a polymer-drug nanoaggregate sample can be dissolved in water, saline, phosphate-buffered saline (PBS), or a solvent as described herein to a predetermined final concentration. The sample is then filtered through a selected filter with a pre-determined filtration surface area, such as, a 25 mm diameter sterile syringe filter assembly having a 0.22 μm filtration pore size, with a predetermined starting volume V₀. A passing volume Vₚ that passed through the filter can be obtained. Filtration rating R_{f} for a sample can be calculated based on formula:

$${R}_{f} = {V}_{p} / {V}_{0} .$$

The filtration rating R_{f} can be expressed as a percentage or a fraction at a pre-determined filtration surface area. In some cases, as disclosed herein, standard sterile filters of 25 mm diameter can be used. The filtration rating R_{f} can be expressed as a percentage or a fraction with a sterile filter of 25 mm diameter that has a pre-determined filtration surface area. Filtration ratings measured using a different size of filters can be converted or normalized in reference to the standard 25 mm diameter filter. A percentage is used in this disclosure. In embodiments of the process, the nanoaggregate can have a filtration rating at least 50%, at least 60%, at least 70%, at least 80%, at least 90% or more; or in a range of 50-100%, 60-100%, 70-100%, 80-100%, 90-100%, 55-100%, 65-100%, 75-100%, 85-100%, 95-100%, 55-95%, 65-95%, 75-95%, 85-95%, 50-95%, 60-95%, 70-95%, 80-95%, 90-95%, 50-90%, 60-90%, 70-90%, 80-90%, 55-90%, 65-90%, 75-90%, 85-90%; or, in any rate, must be 50% or greater.

The pharmaceutical composition disclosed herein can be formulated for parenteral, oral, nasal, transdermal (topical), transmucosal, rectal administration, or a combination thereof and can comprise one or more pharmaceutical suitable carriers. In some cases, the pharmaceutical composition disclosed herein can be formulated for intravenous (IV), intradermal (ID), subcutaneous (SC), oral, transdermal (topical), transmucosal, rectal administration, or a combination thereof. In some cases, the pharmaceutical composition disclosed herein can be formulated for intravenous (IV), intradermal (ID), subcutaneous (SC), transdermal (topical) or transmucosal administration. In some cases, the pharmaceutical composition disclosed herein can be formulated for oral administration, such as, tablets, capsules, oral spray, solutions, or suspensions. In some cases, the pharmaceutical composition disclosed herein can be formulated for nasal administration, such as, nasal spray. The pharmaceutical suitable carriers disclosed herein can be suitable.

In some cases, the pharmaceutical composition can be prepared with a process, the process can comprise the steps of:
(1) forming a nanoaggregate comprising a polymer and at least one bioactive agent that is water insoluble or poorly water soluble by mixing the polymer and the bioactive agent in a nanoaggregate solution comprising at least one organic solvent;
(2) removing the organic solvent from the nanoaggregate to form a dried nanoaggregate;
(3) dissolving the dried nanoaggregate in water, saline or PBS to form an aqueous nanoaggregate solution; and
(4) lyophilizing the aqueous nanoaggregate solution to form a lyophilized nanoaggregate;
wherein the polymer is water soluble and comprises at least one first terminal group modified with H or a hydrophobic moiety and a second terminal group modified with a hydrophilic moiety; and
wherein the first terminal group comprises in a range of from 1% to 99% of H and 1% to 99% of the hydrophobic moiety that can comprise saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof.

The aqueous nanoaggregate solution can be filtered through a 0.22 μm filter to produce a sterilized nanoaggregate prior to lyophilizing.

In some cases, the nanoaggregate can be produced by dissolving the polymer and bioactive agent together in the nanoaggregate solution comprising the organic solvent to form the nanoaggregate. Any organic solvents or a mixture thereof can be suitable. In some cases, the organic solvent can comprise acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxy-, ethane, dimethoxyethane (DME, also known as glyme), dimethyl-, formamide (DMF), dimethyl sulfoxide (DMSO), 1,4-dioxane, ethanol, ethyl acetate, ethylene glycol, glycerin, heptane, hexamethylphosphoramide, (HMPA), hexamethylphosphorous, triamide (HMPT), hexane, methanol, methyl t-butyl ether (MTBE), methylene chloride, N-methyl-2-pyrrolidinone, (NMP), nitromethane, pentane, petroleum ether (ligroin or ligroine), 1-propanol, 2-propanol, pyridine, tetrahydrofuran (THF), toluene, triethyl amine, o-xylene, m-xylene, p-xylene, or a combination thereof.

In some cases, the nanoaggregate can be produced by dissolving the polymer in a first organic solvent to form a polymer solution; dissolving the bioactive agent in a second organic solvent to form bioactive agent solution; and mixing the polymer solution and the bioactive agent solution to form the nanoaggregate; wherein the first organic solvent and the second organic solvent can be the same or different and can be independently selected from the organic solvents disclosed above.

In some cases, the nanoaggregate can be produced by dissolving the polymer in an aqueous solution, a first organic solvent or a combination thereof, to form a polymer solution; dissolving the bioactive agent in a second solvent comprising a second organic solvent to form a bioactive agent solution; and mixing the polymer solution and the bioactive agent solution to form the nanoaggregate; wherein the first organic solvent and the second organic solvent can be the same or different; and wherein the second organic solvent is a water miscible organic solvent. The first solvent can be selected from an aqueous solution, such as, water, saline, a buffer, such as, phosphate-buffered saline (PBS), a first organic solvent or a combination thereof. The first organic solvent and the second organic solvent can be independently selected from a water miscible organic solvent, such as, methanol, ethanol, acetone, propanol, isopropanol, and a combination thereof. In some cases, the first organic solvent and the second organic solvent can independently comprise a mixture of two or more solvents. In some cases, the first organic solvent and the second organic solvent can independently comprise a chloroform/ethanol mixture.

The pharmaceutical composition can have a pH value in a range of from about 3.0 to about 10.0. In some cases, the pharmaceutical composition can have a pH value in a range of from about 7.0 to about 9.0. In some cases, the pharmaceutical composition can have a pH value in a range of from about 7.0 to about 8.0. In some cases, the pharmaceutical composition can have a pH value in a range of from about 7.0 to about 7.5. In some cases, the pharmaceutical composition can have a pH value in a range of from about 3.0 to about 6.9. In some cases, the pharmaceutical composition can have a pH value in a range of from about 3.0 to about 7.0. In some cases, the pharmaceutical composition can have a pH value in a range of from about 4.0 to about 7.5. In some cases, the pharmaceutical composition can have a pH value in a range of from about 3.0 to about 6.9, 4.0 to about 6.9, or 5.6 to about 6.9. The pharmaceutical composition can be adjusted with an acid or a base to arrive at the desired pH range. An acid, such as, HCl or other acids can be suitable. A base, such as, NaOH, or other bases, can be suitable.

The nanoaggregate of the pharmaceutical composition produced by the process of this invention can be free from human serum albumin, organic solvent, detergent, or oil, as described above. The nanoaggregate of the pharmaceutical composition produced by the process of this invention can be free from human serum albumin, organic solvent, detergent, oil or free acid. In a further example, the pharmaceutical composition produced by the process of this invention can be free from human serum albumin, organic solvent, detergent, oil or free acid, as described above.

The process disclosed above and hereafter can further comprise mixing an additional bioactive agent that is formulated free from the polymer in the pharmaceutical composition. In some cases, a chemical or small molecule drug, chemotherapy drugs, inorganic-based drug, biological or large molecule-based drug, modifications or derivatives thereof, and combinations thereof, formulated free from the polymer mentioned above, individually or in a combination thereof, can be suitable.

In some cases, this disclosure is further directed to a method for treating or preventing a disease of a subject in need thereof, the method can comprise administering the subject with an effective dose of a pharmaceutical composition comprising:
a nanoaggregate comprising a polymer and at least one bioactive agent that is water insoluble or poorly water soluble; and
optionally a pharmaceutical suitable carrier;
wherein the pharmaceutical composition is soluble in an aqueous solution to produce at least 1 mg/mL of the bioactive agent in the aqueous solution;
wherein the polymer is water soluble; and
wherein the polymer can comprise:
a first polymer comprising at least one first terminal group modified with H or a hydrophobic moiety and a second terminal group modified with a hydrophilic moiety, wherein the first terminal group comprises in a range of from 1% to 99% of H and 1% to 99% of the hydrophobic moiety that comprises saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or
a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or
a combination thereof.

In some cases, the polymer can comprise the first polymer, as disclosed herein.

In some cases, the polymer can consist of the first polymer, as disclosed herein. In some cases, the pharmaceutical composition can be free from the polymers selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

In some cases, the polymer can comprise a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

In some cases, the polymer can comprise the first polymer and one or more subsequent polymers (also referred to as “second polymer”) selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

The bioactive agents disclosed herein can be suitable as pharmaceutic or pharmacologic agents.

Any polymers disclosed herein can be suitable. In some cases, the polymer can comprise a polyoxazoline (POX) that comprises a linear portion, a branched portion, or a combination thereof, and wherein the polyoxazoline (POX) comprises poly(2-oxazoline), poly(2-methyloxazoline), poly(2-ethyloxazoline), poly(2-propyloxazoline), poly(isopropyloxazoline), or a combination thereof. In some cases, the polyoxazoline is poly(2-ethyloxazoline).

In some cases, the polyoxazoline can comprise a molar ratio of monomer to initiator in a range of from 50:1 to 80:1.

The nanoaggregate can be of a size in a range of less than about 120 nm before lyophilization. In some cases, the nanoaggregate can be of a size in a range of from about 50 nm to about 120 nm before lyophilization. In some cases, the nanoaggregate can have a weight ratio of the polymer to the bioactive agent in a range of from about 2:1 to about 200:1.

In some cases, the method disclosed herein, the pharmaceutical composition can comprise two or more subsequent bioactive agents. In some cases, at least one of the two or more bioactive agents is paclitaxel. In some cases, at least one of the two or more bioactive agents is rapamycin. In some cases, the pharmaceutical composition can comprise paclitaxel and one or more additional bioactive agents disclosed herein that is different from paclitaxel. In some cases, the pharmaceutical composition can comprise rapamycin and one or more subsequent bioactive agents selected from a protein, a peptide, an antibody, a fragment of an antibody, a chemical compound, a small molecule drug, one or more chemotherapy drugs, a vaccine, and a combination thereof. In some cases, the one or more subsequent bioactive agents can be selected from a gemcitabine, a taxane, paclitaxel, docetaxel, cabazitaxel, larotaxel, milataxel, ortataxel, tesetaxel, Topoisomerase 1 (Top 1) inhibitor, camptothecin derivative, innotecan (CPT-11), SN-38, topotecan, Topoisomerase 2 (Top 2) inhibitor, doxorubicin, etoposide, ciprofloxaxin, a platinum-based antineoplastic agent, anti-programmed cell death protein (PD)1 antibodies, anti-PD ligand (PD-L)1 antibodies, anti-CTLA-4 (cytotoxic T lymphocyte-associated antigen) antibodies, anti-LAG3 (lymphocyte activation gene-3) antibodies, anti-TIM-3 (T cell immunoglobulin and mucin domain-3) antibodies, anti-CD19 antibodies, anti-CD20 antibodies, cytokines, interleukins, interferon α2a, interferon α, granulocyte colony stimulating factor (G-CSF), Neupogen or Filgrastim, T-cell receptor (TCR), chimeric antigen receptor or chimeric antigen T-cell receptor (CAR-T), an STING protein, STING agonists, STING activators, STING inhibitors, STING antagonists or a combination thereof, indoleamine 2,3 dioxygenase (IDO) inhibitors, indoleamine 2,3-dioxygenase 1 (IDO1) inhibitors, a vaccine, and a combination thereof. In some cases, at least one of the two or more bioactive agent can comprise STING modulating molecules, such as, benzimidazole compounds disclosed in patent publications WO2017175156A1 and WO2020156363, pyridinyl imidazole compounds disclosed in patent publications WO2019134705, WO2020010451 and US20200031825, or a combination.

In some cases, the method disclosed herein, the pharmaceutical composition can comprise one or more additional bioactive agents selected from gemcitabine, taxane, paclitaxel, temozolomide, platinum-based antineoplastic agent, daunorubicin, doxorubicin, epirubicin, mitomycin, methotrexate, fluorouracil, carboplatin, carmustine, methyl-CCNU, cisplatin, vinorelbine, capecitabine and a combination thereof. In some cases, the additional bioactive agent can comprise 7-ethyl-10-hydroxycamptothecin (SN-38).

In some cases, the nanoaggregate can be of a size less than 150 nm before lyophilization. In some cases, the nanoaggregate can be of a size less than 120 nm before lyophilization. In some cases, the nanoaggregate can be of a size in a range of from about 50 nm to about 150 nm before lyophilization. In some cases, the size of the nanoaggregates or nanoparticles, can range from about 50 to about 100 nm before lyophilization. In some cases, the size of the nanoaggregates or nanoparticles, can range from about 50 to about 120 nm before lyophilization. Particle size can be measured by light scattering.

In some cases, the nanoaggregate can have a weight ratio of the polymer to rapamycin in a range of from about 2:1 to about 20:1.

In some cases, the nanoaggregate can have has a weight ratio of the polymer to rapamycin in a range of from about 5:1 to about 8:1.

In some cases, the nanoaggregate can further comprise a targeting moiety comprises an antibody, an antigen-binding portion thereof, an antigen, a cell receptor, a cell receptor ligand, a ligand for a cellular protein, a ligand for a membrane protein, a small molecule ligand, a lectin ligand, or a combination thereof.

In some cases, the nanoaggregate can be free from human serum albumin, organic solvent, detergent, or oil. In some cases, the nanoaggregate can be free from human serum albumin, organic solvent, detergent, oil or free acid. In some cases, the nanoaggregate can be free from human serum albumin. In some cases, the nanoaggregate can be free from organic solvent. In some cases, the nanoaggregate can be free from detergent. In some cases, the nanoaggregate can be free from oil. In some cases, the nanoaggregate can be free from free acid. In some cases, the nanoaggregate can be free from materials selected from the group consisting of human serum albumin, organic solvent, detergent, oil, free acid, and a combination thereof.

In some cases, the pharmaceutical composition can be free from human serum albumin, organic solvent, detergent, or oil. In some cases, the pharmaceutical composition can be free from human serum albumin, organic solvent, detergent, oil or free acid. In some cases, the pharmaceutical composition can be free from human serum albumin. In some cases, the pharmaceutical composition can be free from organic solvent. In some cases, the pharmaceutical composition can be free from detergent. In some cases, the pharmaceutical composition can be free from oil. In some cases, the pharmaceutical composition can be free from free acid. In some cases, the pharmaceutical composition can be free from materials selected from the group consisting of human serum albumin, organic solvent, detergent, oil, free acid, and a combination thereof.

In some cases of the method disclosed herein, in a range of from 1% to 100% of the second terminal group is free from primary amine. In some cases, in a range of from 1% to 100% of the second terminal group comprises hydroxyl group. All percentages are based on the total number of the second terminal group.

In some cases, the pharmaceutical composition can have a pH value in a range of from 3.0 to 6.9.

In some cases, suitable to the method, the bioactive agent can comprise at least a compound having Formula (1)-Formula (29) (FIG. 12A-FIG. 12E), a pharmaceutically acceptable salt thereof, solvate thereof, prodrug thereof, isomer thereof, or a combination thereof.

In some cases, the bioactive agent can comprise a compound having Formula (1)

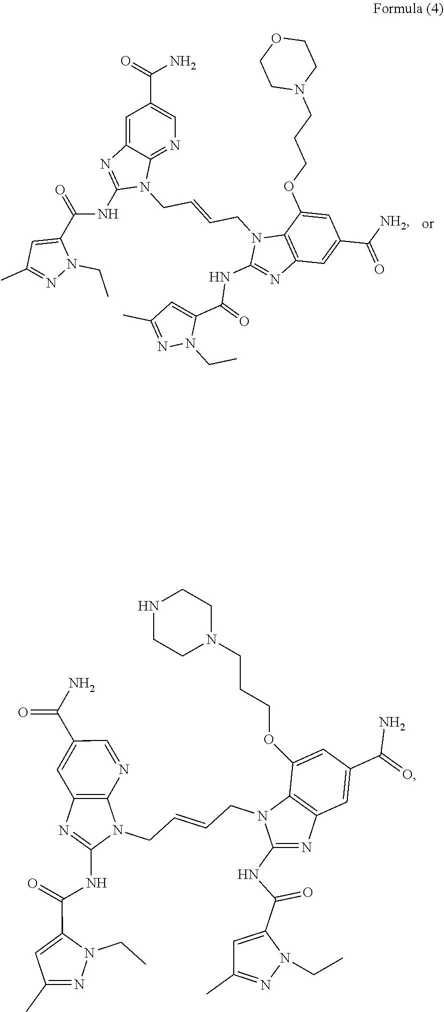 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

a pharmaceutically acceptable salt thereof, solvate thereof, prodrug thereof, isomer thereof, or a combination thereof.

In some cases, the disease can be selected from one or more immune disorders, infectious diseases, cancers, and a combination thereof.

In some cases, immune disorders can include aforementioned immunodeficiency disorders, overactive immune disorders, autoimmune diseases, and other disorders or symptoms that have abnormal immune systems.

In some cases, the method can further comprise the step of administering the subject with one or more subsequent bioactive agents selected from a protein, a peptide, an antibody, a fragment of an antibody, a chemical compound, a small molecule drug, one or more chemotherapy drugs, a vaccine, and a combination thereof. In some cases, the one or more subsequent bioactive agents can be selected from a gemcitabine, a taxane, paclitaxel, docetaxel, cabazitaxel, larotaxel, milataxel, ortataxel, tesetaxel, Topoisomerase 1 (Top 1) inhibitor, camptothecin derivative, irinotecan (CPT-11), SN-38, topotecan, Topoisomerase 2 (Top 2) inhibitor, doxorubicin, etoposide, ciprofloxaxin, a platinum-based antineoplastic agent, anti-PD1 antibodies, anti-PD-L1 antibodies, anti-CTLA-4 (cytotoxic T lymphocyte-associated antigen) antibodies, anti-LAG3 (lymphocyte activation gene-3) antibodies, anti-TIM-3 (T cell immunoglobulin and mucin domain-3) antibodies, anti-CD19 antibodies, anti-CD20 antibodies, cytokines, interleukins, interferon α2a, interferon α, granulocyte colony stimulating factor (G-CSF), Neupogen or Filgrastim, T-cell receptor (TCR), chimeric antigen receptor or chimeric antigen T-cell receptor (CAR-T), a vaccine, and a combination thereof. Any of bioactive agents disclosed herein can be suitable.

In some cases, each of the one or more subsequent bioactive agents can be administered to the subject, prior to, at the same time as, or after administering the pharmaceutical composition. Each of the one or more subsequent bioactive agents can be administered to the subject independently. In some cases, the subsequent bioactive agent can comprise 7-ethyl-10-hydroxycamptothecin (SN-38).

The pharmaceutical composition can be administered to the subject via intravenous (IV) injection, subcutaneous (SC) injection, intramuscular (IM) injection, intradermal (ID) injection, or a combination thereof. A combination of intravenous (IV) injection, subcutaneous (SC) injection, intramuscular (IM) injection, or intradermal (ID) injection can also be suitable.

In some cases, the pharmaceutical composition can be an adjuvant for a vaccine. In some cases, the pharmaceutical composition is a prophylactic vaccine, a therapeutic vaccine, or a combination thereof, wherein the pharmaceutical composition further comprises at least one immune agent for stimulating immune response in a subject in need thereof. In some cases, the pharmaceutical composition is selected for treating or preventing at least one infectious disease selected from Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, and a combination thereof.

In some cases, the disease can be one or more cancers comprising adenocarcinoma of the stomach or lower esophagus, AIDS-related Kaposi's sarcoma, Ampullary Carcinoma, angiosarcoma, B cell lymphoma, bile duct cancer, bladder cancer, brain cancer, breast cancer, cervical cancer, cervical carcinoma, cholangiocarcinoma, colon cancer, epithelial cancer, esophageal cancer, esophageal carcinoma, gastric cancer, genitourinary cancer, glioblastoma, head and neck cancer, head and neck carcinoma, head and neck squamous cell carcinoma (HNSCC), hematopoietic cancers, testicular cancer, colon and rectal cancers, Hodgkin lymphoma, Hodgkin's Disease, hormone-refractory prostate cancer, kidney cancer, large bowel cancer, liver cancer, lymphoma, melanoma, metastatic breast cancer, metastatic pancreas cancer, mycosis fungoides, myeloid leukemia, nasopharyngeal carcinoma, nervous system cancer, neuroblastoma, non-small cell lung cancer (NSCLC), Oral Adenoid Cystic Carcinoma, ovarian cancer, pancreatic cancer, prostate cancer, prostatic cancer, pulmonary cancer, renal cancer, Sinonasal Squamous Cell Carcinoma (SCC), skin cancer, small cell lung cancer (SCLC), squamous cell carcinoma of head and neck, squamous cell carcinoma of the head and neck cancer (SCCHN), stage IIB-IV melanoma, T cell lymphoma, triple negative breast cancer (TNBC), upper GI adenocarcinoma, urothelium transitional cell carcinoma, or other cancer or cancers including those cancer or neoplastic disorder or disease to be diagnosed or recognized. The disease can be any of the cancers disclosed above or hereafter. In some cases, the cancer can be a solid tumor. In some cases, the cancer can be one or more cancers of the blood.

In some cases, the disease can be one or more cancers comprising Ampullary Carcinoma, Adrenal Gland Cancer, breast cancers, ovarian cancers, lung cancers, NSCLC (Non-Small Cell Lung Cancer), cholangiocarcinoma, small cell lung cancer, biliary track cancer, bladder cancer, cervical cancer, soft tissue sarcoma, uterine sarcoma, colon cancers, gastric cancers, melanoma, nasopharyngeal carcinoma, Neuroendocrine cancer, head and neck cancers, Oral Adenoid Cystic Carcinoma, pancreatic cancers, Sinonasal Squamous Cell Carcinoma (SCC), thyroid cancer, one or more metastatic cancers derived therefrom, or a combination thereof.

In some cases, the one or more cancers are selected from thyroid cancer, relapsed ER(+) high-grade ovarian cancer, ER(+) locally advanced or metastatic breast cancer, advanced neuroendocine tumor, diffuse large B cell lymphoma, advanced solid tumors, metastatic clear cell renal cancer, relapsed or refractory non-Hodgkin lymphoma, chronic lymphocytic lymphoma, recurrent endometrial cancer, perivascular epithelioid cell tumor (PEComa), advanced unresectable or metastatic malignant perivascular epithelioid cell tumor, ovarian cancers, lung cancers, NSCLC (Non-Small Cell Lung Cancer), small cell lung cancer, biliary track cancer, bladder cancer, cervical cancer, soft tissue sarcoma, uterine sarcoma, colon cancers, gastric cancers, melanoma, head and neck cancers, pancreatic cancers, one or more metastatic cancers derived therefrom, and a combination thereof. In some cases, the one or more cancers can be advanced unresectable or metastatic malignant perivascular epithelioid cell tumor (PEComa).

As used herein, a subsequent bioactive agent can have a molecular weight in a range of from about 10 to 1,000,000 in one example, 100 to 500,000 in another example, 100 to 200,000 in yet another example, 500 to 200,000 in yet another example, 1,000 to 200,000 in yet another example, 5,000 to 200,000 in yet another example, 10,000 to 200,000 in yet another example, 15,000 to 200,000 in yet another example, 20,000 to 200,000 in yet another example, and 25,000 to 200,000 in yet another example. A bioactive agent can also have a molecular weight in a range of from about 100 to 100,000 in one example, 100 to 75,000 in yet another example, 100 to 50,000 in yet another example, 100 to 30,000 in yet another example, and 100 to 25,000 in yet another example.

A subsequent bioactive agent can comprise a drug, protein, recombinant protein, antibody, Fab antibody fragments, other antibody fragments that bind antigen, enzymes, viruses, virus fragments and a combination thereof. A subsequent bioactive agent can be selected from a peptide, a monoclonal antibody, a fragment of a monoclonal antibody, a polyclonal antibody, a fragment of a polyclonal antibody, a synthetic antibody, a fragment of a synthetic antibody, or a combination thereof. A subsequent bioactive agent can comprise, for example, antibodies or antigen-binding portions thereof, such as, alemtuzumab, bevacizumab, cetuximab, ibritumomab, rituximab, trastuzumab, gemtuzumab, anti-PD1 antibodies (such as, Keytruda or pembrolizumab, Opdivo or nivolumab, Bavencio or avelumab, Imfinzi or durvalumab, Tecentriq or atezolizumab), anti-PD-L1 antibodies, anti-CTLA-4 (cytotoxic T lymphocyte-associated antigen, also known as CD152) antibodies, anti-LAG3 (lymphocyte activation gene-3) antibodies, anti-TIM-3 (T cell immunoglobulin and mucin domain-3) antibodies, anti-CD19 antibodies, anti-CD20 antibodies, such as, tositumomab, cytokines, such as, interleukins, interferon α2a, interferon α, granulocyte colony stimulating factor (G-CSF) or Neupogen also known as Filgrastim, T-cell receptor (TCR), chimeric antigen receptor or chimeric antigen T-cell receptor (CAR-T), an STING protein, STING agonists, STING activators, STING inhibitors, STING antagonists, STING modulating molecules, indoleamine 2,3 dioxygenase (IDO) inhibitors, indoleamine 2,3-dioxygenase 1 (IDO1) inhibitors, peptide hormones, such as, insulin, glucagon, glucagon like peptide-1, erythropoietin (EPO), thyroperoxidase (TPO), follicle stimulating hormone and so on, ligands of cell surface receptors, lectins, nucleic acids, such as, siRNA's, ribozymes, antisense nucleic acids, naked nucleic acids and so on, viruses, virus-like particles and the like. Examples can include Ecallantide.

In some cases, the method can comprise administering the subject with an effective dose of the pharmaceutical composition comprising the nanoaggregate comprising the polymer and the bioactive agent, and administering one or more subsequent bioactive agents to the subject, prior to, at the same time as, or after administering the pharmaceutical composition. Any of the subsequent bioactive agent disclosed herein can be suitable. In some cases, the bioactive agent can be selected from alemtuzumab, bevacizumab, cetuximab, ibritumomab, rituximab, trastuzumab, gemtuzumab, anti-PD1 antibodies (such as, Keytruda or pembrolizumab, Opdivo or nivolumab, Bavencio or avelumab, Imfinzi or durvalumab, Tecentriq or atezolizumab), anti-PD-L1 antibodies, anti-CTLA-4 (cytotoxic T lymphocyte-associated antigen, also known as CD152) antibodies, anti-LAG3 (lymphocyte activation gene-3) antibodies, anti-TIM-3 (T cell immunoglobulin and mucin domain-3) antibodies, anti-CD19 antibodies, anti-CD20 antibodies, such as, tositumomab, one or more cytokines, interferon α2a, interferon α, granulocyte colony stimulating factor (G-CSF), Neupogen also known as Filgrastim, T-cell receptor (TCR), chimeric antigen receptor or chimeric antigen T-cell receptor (CAR-T), nucleic acids, such as, siRNAs, ribozymes, antisense nucleic acids, naked nucleic acids and so on, viruses, virus-like particles and a combination thereof. In some cases, the bioactive agent can be selected from alemtuzumab, bevacizumab, cetuximab, ibritumomab, rituximab, trastuzumab, gemtuzumab, Keytruda (pembrolizumab), Opdivo (nivolumab), Bavencio (avelumab), Imfinzi (durvalumab, Tecentriq or atezolizumab), anti-PD-L1 antibodies, anti-PD-L1 antibodies, anti-CTLA-4 antibodies, anti-LAG3 antibodies, anti-TIM-3 antibodies, anti-CD19 antibodies, anti-CD20 antibodies, T-cell receptor (TCR), chimeric antigen T-cell receptor (CAR-T), and a combination thereof.

A subsequent bioactive agent can comprise a vaccine. A vaccine can comprise an antigen, a toxin, a modified or disabled toxin including natural or synthetic molecules that can cause immunoreaction in a biosystem, such as, in humans or animals. A vaccine can be attached to the polymer via covalent bonds, non-covalent linkages, or a combination thereof. Commercial vaccines and the vaccines listed by US Centers for Disease Control and Prevention (CDC) can be suitable.

A subsequent bioactive agent described herein can include any chemical or small molecule drug, chemotherapy drugs, inorganic-based drug, biological or large molecule-based drug, modifications or derivatives thereof, and combinations thereof. Chemotherapy drugs listed by NIH National Cancer Institute (https://www.cancer.gov/about-cancer/treatment/drugs) or any future updates can be suitable.

A subsequent bioactive composition can be administered with intravenous (IV), intramuscular (IM), subcutaneous (SC) or intradermal (ID) injections, orally, through inhalation, nasally, through an eye, for example, using drops or an ointment, transdermally, for example, using a patch, or a combination thereof. A combination of any of aforementioned administering routes can also be suitable.

A nanoaggregate disclosed herein can be a nanocomposite, nanoparticle of one or more materials or components, for example, a polymer alone, a polymer and a bioactive agent, wherein at least one dimension of the physical mixture is at nanometer range as defined herein. In the instant disclosure, such a mixture can contain different nanoscale phases or domains formed between a bioactive agent and a branched homopolymer molecule in either solid or liquid state. Nanocomposites can include a combination of a bulk matrix (e.g., branched homopolymers and rapamycin) and nanodimensional phase(s), which may exhibit different properties due to dissimilarities of structure and chemistry (e.g., the domain formed by rapamycin and the surface groups of branched polymer, as well as the domains formed by the interior of the branched polymers). Since the solubility of the domains/phases may be different, on dissolving the nanocomposite in an aqueous solution, one of the phases may dissolve faster than the other or others, resulting in a gradual breakdown of the composite nanoaggregate leading to a graded and controlled release of the composite components and optionally, reformation of one or more of the components into a novel form, such as, a new nanoaggregate. The terms, “nanocomposite,” “nanoparticle”, “nanoaggregate”, “nanoaggregates”, “aggregate” and “aggregates” are equivalent and are used interchangeably herein.

The size of the nanoaggregate described in the disclosure ranges from about 10 to about 500 nm in diameter in one example, from about 30 nm to about 300 nm in diameter in another example, from 50 to 150 nm in yet another example, from 50 to 120 nm in yet another example, from 50 to 100 nm in yet another example, and from 70 to 90 nm in a further example, before lyophilization. Nanoaggregates may exhibit size-related properties that differ significantly from those observed for microparticles.

Applicants discovered, when a polymer synthesized using a monomer to initiator molar ratio in a range of from 50:1 to 80:1, such as, of 60:1, is mixed with rapamycin at a polymer to rapamycin weight ratio of 5;1 to 7:1, the nanoparticles formed are in a range of from 50 to 150 nm in size before lyophilization, which allows the particles to pass through a 0.22 μm filter with little difficulty.

The nanoaggregate can have a filtration rating through a 0.22 μm filter in a range of from 50 to 100 percent. In some cases, polyoxazoline polymers with a specific range of monomer to initiator molar ratios, such as, polymers having monomer to initiator molar ratios in a range of from 50:1 to about 80:1, such as, H-PEOXABP60, C₁₈PEOXABP60, H/C₁₈PEOXABP60, or a combination thereof disclosed herein, can be suitable.

As disclosed above and hereafter, the nanoaggregates can be linked with a targeting moiety or group including, but not limited to, an antibody (or antigen-binding portion thereof), antigen, cognate carbohydrates (e.g., sialic acid), a cell surface receptor ligand, a moiety that binds a cell surface receptor, such as, prostate-specific membrane antigen (PSMA), a moiety that binds a cell surface saccharide, an extracellular matrix ligand, a cytosolic receptor ligand, a growth factor, a cytokine, an incretin, a hormone, a lectin, a lectin target, such as, a galactose, a galactose derivative, an N-acetylgalactosamine, a mannose, a mannose derivative and the like, a vitamin, such as, a folate, a biotin and the like, an avidin, a streptavidin, a neutravidin, etc., to form a conjugate so that the targeting group(s) are incorporated with nanocomposite particle of interest (FIG. 10A-10B).

In some cases, the bioactive agent can be dissolved in methanol or ethanol in various amounts of up to 40 mg/mL. A hydrocarbon (CH₃(CH₂)₁₇)-modified randomly branched PEOX60 (monomer to initiator molar ratios=60:1) (herein referred to as C₁₈PEOXABP60) can be prepared as taught in PCT Publication No.: WO2014/123791, herein incorporated by reference in entirety, and dissolved at varying concentrations of up 100 mg/mL in methanol or ethanol. The two solutions then can be mixed in various volumes to result in final homopolymer to the bioactive agent weight ratios in the mixtures ranging from 2:1 to 20:1 to form the nanoaggregate. The nanoaggregate can be rotary evaporated to dryness to form a dried nanoaggregate. The dried nanoaggregate then can be re-dissolved or suspended in water or saline, followed by sterile filtration with a 0.22 μm filter and lyophilization for 20 to 72 hours depending on volume to yield a lyophilized nanoaggregate or dry powder.

In some cases, a polymer mixture of H/C₁₈PEOXABP60 can be suitable. The polymer mixture H/C₁₈PEOXABP60 can comprise polymers having the second terminal group modified by —OH, NH₂ or a combination thereof.

The size of the nanoaggregates or nanoparticles, as measured by light scattering, can range from about 50 to about 150 nm in one example, about 50 to about 100 nm in one example, from about 60 to about 100 nm in another example, from about 70 to about 100 nm in yet another example and from about 70 to 95 nm in yet another example before lyophilization. In some cases, the size of the nanoaggregates or nanoparticles, as measured by light scattering, can range from about 50 to about 100 nm before lyophilization. The pharmaceutical composition can comprise the nanoaggregate is of a size in a range of from about 50 to about 150 nm in diameter after lyophilization. The size can be measured by reconstitution of the dried nanoaggregate in a saline, a sodium bicarbonate solution, water, buffer or a combination thereof. The size of the nanoaggregate can be measured by reconstitution of the dried nanoaggregate in a buffer, such as, a phosphate-buffered saline (PBS) in one example, in a combination of PBS and saline (sodium chloride) in another example, and in a combination of saline and sodium bicarbonate in a further example.

A pharmaceutical composition comprising the nanoaggregate disclosed herein can be formulated to be compatible with the intended administering route and can comprise one or more pharmaceutical suitable carriers. Examples of routes of administration include parenteral, e.g., intravenous (IV), intradermal (ID), subcutaneous (SC), oral (e.g., inhalation), transdermal (topical), transmucosal and rectal administration. Solutions or suspensions used for parenteral, intradermal or subcutaneous application can include one or more pharmaceutical suitable carriers, such as, a sterile diluent, such as, water for injection, saline, oils, polyethylene glycols, glycerine, propylene glycol or other synthetic solvents; antibacterial agents, such as, benzyl alcohol or methyl parabens; antioxidants, such as, ascorbic acid or sodium bisulfite; chelating agents, such as, EDTA; buffers, such as, acetates, citrates or phosphates; and agents for the adjustment of tonicity, such as, sodium chloride or dextrose. pH can be adjusted with acids or bases, such as HCl or NaOH. The parenteral preparation can be enclosed in ampoules, disposable syringes or multiple dose vials made of glass or plastic as an article of manufacture. The pharmaceutical composition can be packaged in a container, pack or dispenser together with instructions for administration.

The pharmaceutical compositions can be suitable for injectable use and can include sterile aqueous solutions or dispersions and sterile powders for the extemporaneous preparation of sterile injectable solutions or dispersions. For intravenous administration, suitable carriers can include physiological saline, bacteriostatic water, Cremophor EL® (BASF; Parsippany, N.J.) or phosphate-buffered saline (PBS). The composition is sterile and is fluid to the extent that syringability exists. The composition must be stable under the conditions of manufacture and storage and must be preserved against the contaminating action of microorganisms, such as, bacteria and fungi. The pharmaceutical composition can comprise one or more solvents or dispersion medium containing, for example, water, ethanol, polyol (for example, glycerol, propylene glycol, liquid PEG, polysorbates and the like) and suitable mixtures thereof.

Some pharmaceutical suitable carriers can be used for maintaining proper fluidity of the composition, for example, by the use of a coating such as, lecithin, by the maintenance of the required particle size in the case of dispersion, use of a thickener and the use of surfactants. Further pharmaceutically suitable carrier can include various antibacterial and antifungal agents, for example, parabens, chlorobutanol, phenol, ascorbic acid and the like, to prevent or inhibit growth or action of microorganisms. Isotonic agents, for example, sugars, polyalcohols, such as, mannitol, sorbitol or sodium chloride, can be included in the composition as a pharmaceutically suitable carrier. An agent that delays absorption, for example, aluminum monostearate or gelatin can also be used as a pharmaceutically suitable carrier.

In a further embodiment, the pharmaceutical composition can comprise one or more pharmaceutical suitable carriers that will protect the compound against rapid elimination from the body of a subject, such as, a controlled release formulation, including implants and microencapsulated delivery systems. Biodegradable, biocompatible polymers can be used, such as, ethylene vinyl acetate, polyanhydrides, polyglycolic acid, collagen, polyorthoesters and polylactic acid. Methods for preparation of such formulations will be apparent to those skilled in the art. The materials also can be obtained commercially, for example, from Alza Corporation and Nova Pharmaceuticals, Inc.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches or capsules. Oral compositions also can be prepared using a fluid carrier to yield a syrup or liquid formulation, or for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients or compounds of a similar nature: a binder, such as, microcrystalline cellulose, gum tragacanth or gelatin; an excipient, such as, starch or lactose; a disintegrating agent, such as, alginic acid, Primogel or corn starch; a lubricant, such as, magnesium stearate or Sterotes; a glidant, such as, colloidal silicon dioxide; a sweetening agent, such as, sucrose or saccharin; or a flavoring agent, such as, peppermint, methyl salicylate or orange flavoring.

For administration by inhalation, the compound is delivered in the form of, for example, an aerosol spray from a pressurized container or dispenser that contains a suitable propellant, e.g., a gas, such as, carbon dioxide or a nebulizer, or a mist.

Systemic administration also can be by transmucosal or transdermal means. For transmucosal or transdermal administration, penetrants appropriate to the barrier to be permeated are used in the formulation. Such penetrants generally are known in the art and include, for example, for transmucosal administration, detergents, bile salts and fusidic acid derivatives. Transmucosal administration can be accomplished through the use of nasal sprays or suppositories. For transdermal administration, the active compounds are formulated into ointments, salves, gels or creams as generally known in the art. Another known penetrant is dimethyl sulfoxide.

The compound also can be prepared in the form of suppositories (e.g., with conventional suppository bases, such as, cocoa butter and other glycerides) or retention enemas for rectal delivery.

It can be advantageous to formulate oral or parenteral compositions in dosage unit form for ease of administration and uniformity of dosage. Dosage unit form as used herein refers to physically discrete units suited as unitary dosages for a subject to be treated; each unit containing a predetermined quantity of active compound calculated to produce a desired therapeutic endpoint. The dosages, for example, preferred route of administration and amounts are obtainable based on empirical data obtained from preclinical and clinical studies, practicing methods known in the art. The dosage and delivery form can be dictated by and can be dependent on the characteristics of the bioactive agent, the polymer, the particular therapeutic effect to be achieved, the characteristics and condition of the recipient and so on. For repeated administrations over several days or longer, depending on the condition, the treatment can be sustained until a desired endpoint is attained.

In some cases, this disclosure is directed to a nanoaggregate comprising a polymer and at least one bioactive agent that is water insoluble or poorly water soluble;
wherein the nanoaggregate is soluble in an aqueous solution to produce at least 1 mg/mL of the bioactive agent in the aqueous solution;
wherein the polymer is water soluble;
wherein the bioactive agent comprises a natural or synthetic small molecule-based drug, inorganic-based drug, biological drug, natural or synthetic large molecule-based drug, a derivative thereof, or a combination thereof; and
wherein the polymer comprises:
a first polymer comprising at least one first terminal group modified with H or a hydrophobic moiety and a second terminal group modified with a hydrophilic moiety, wherein the first terminal group comprises in a range of from 1% to 99% of H and 1% to 99% of the hydrophobic moiety that comprises saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or
a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

In some cases, in a range of from about 1% to 100% of the second terminal group can be free from primary amine. In some cases, the nanoaggregate disclosed herein, in a range of from 1% to 100% of the second terminal group can comprise hydroxyl group. All percentages are based on the total number of the second terminal groups.

In some cases, the nanoaggregate can be of a size less than 150 nm before lyophilization. In some cases, the nanoaggregate can be of a size less than 120 nm before lyophilization. In some cases, the nanoaggregate can be of a size in a range of from about 50 nm to about 120 nm before lyophilization.

In some cases, the poly(2-ethyloxazoline) can comprise a molar ratio of monomer to initiator in a range of from 50:1 to 80:1.

In some cases, the nanoaggregate can have a weight ratio of the polymer to the bioactive agent in a range of from about 2:1 to about 200:1. In some cases, the nanoaggregate can have a weight ratio of the polymer to the bioactive agent in a range of from about 5:1 to about 8:1.

In some cases, the nanoaggregate can further comprise a targeting moiety comprises an antibody, an antigen-binding portion thereof, an antigen, a cell receptor, a cell receptor ligand, a ligand for a cellular protein, a ligand for a membrane protein, a small molecule ligand, a lectin ligand, or a combination thereof.

The nanoaggregate can be free from human serum albumin, organic solvent, detergent, or oil. The nanoaggregate can be free from human serum albumin, organic solvent, detergent, oil or free acid.

In some cases, the bioactive agent can comprise any one of the bioactive agents listed above and hereafter. In some cases, the bioactive agent can comprise two or more of the bioactive agents listed above and hereafter. In some cases, the bioactive agent can comprise a natural or synthetic small molecule-based drug, inorganic-based drug, biological drug, natural or synthetic large molecule-based drug, a derivative thereof, or a combination thereof. In some cases, the bioactive agent can comprise taxane, paclitaxel, docetaxel, cabazitaxel, larotaxel, milataxel, ortataxel, tesetaxel, Topoisomerase 1 (Top 1) inhibitor, camptothecin derivative, irinotecan (CPT-11), SN-38, topotecan, Topoisomerase 2 (Top 2) inhibitor, doxorubicin, etoposide, ciprofloxaxin, mTOR inhibitor, at least one STING polypeptide or a part thereof, a nucleic acid encoding the STING polypeptide or a part thereof, a STING inhibitor, a STING activator, a STING agonist, a STING antagonist, a STING modulating molecule, an IDO inhibitor, an IDO1 inhibitor, or a combination thereof, wherein the mTOR inhibitor comprises everolimus, rapamycin, temsirolimus, zotarolimus, torin-1, torin-2, vistusertib, ridaforolimus, one or more dual PI3K-mTOR inhibitors, one or more ATP-competitive mTORC1/2 inhibitors, a derivative thereof, or a combination thereof.

In some cases, the bioactive agent can comprise 7-ethyl-10-hydroxycamptothecin (SN-38).

In some cases, suitable to the nanoaggregate disclosed herein, the bioactive agent can comprise at least a compound having Formula (1)-Formula (29) (FIG. 12A-FIG. 12E), a pharmaceutically acceptable salt thereof, solvate thereof, prodrug thereof, isomer thereof, or a combination thereof.

In some cases, the bioactive agent can comprise a compound having Formula (1):

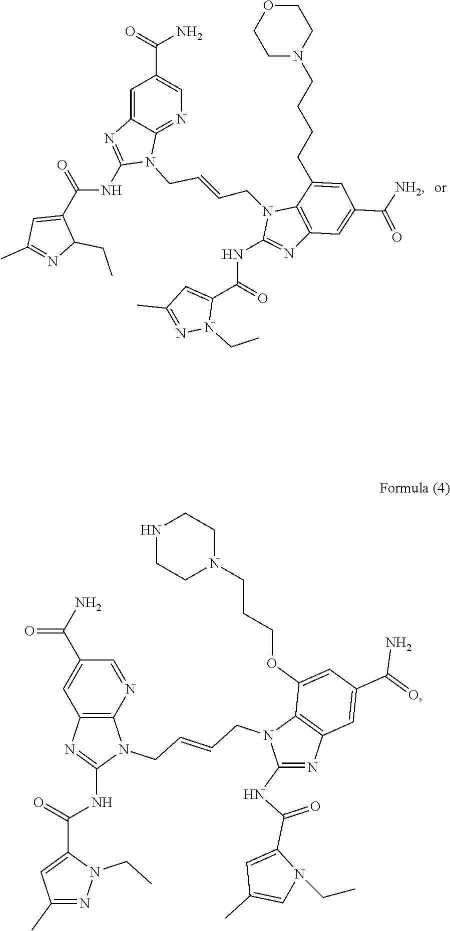 [SMILES: CCC1N=C(C)C=C1C(=O)Nc1nc2cc(C(N)=O)cnc2n1C/C=C/Cn1c(NC(=O)c2cc(C)nn2CC)nc2cc(C(N)=O)cc(CCCCN3CCOCC3)c21 ; CCn1cc(C)cc1C(=O)Nc1nc2cc(C(N)=O)cc(OCCCN3CCNCC3)c2n1C/C=C/Cn1c(NC(=O)c2cc(C)nn2CC)nc2cc(C(N)=O)cnc21]

a pharmaceutically acceptable salt thereof, solvate thereof, prodrug thereof, isomer thereof, or a combination thereof.

This invention is further directed to a use of nanoaggregates comprising a polymer and at least one bioactive agent that is water insoluble or poorly water soluble, and optionally a pharmaceutical suitable carrier, for the manufacturing of a medicament for the treatment of a disease;
wherein the disease is selected from one or more immune disorders, infectious diseases, cancers, and a combination thereof;
wherein the nanoaggregates are soluble in an aqueous solution to produce at least 1 mg/mL of the bioactive agent in the aqueous solution;
wherein the polymer is water soluble; and
wherein the bioactive agent comprises a natural or synthetic small molecule-based drug, inorganic-based drug, biological drug, natural or synthetic large molecule-based drug, a derivative thereof, or a combination thereof;
wherein the polymer comprises:
a first polymer comprising at least one first terminal group modified with H or a hydrophobic moiety and a second terminal group modified with a hydrophilic moiety, wherein the first terminal group comprises in a range of from 1% to 99% of H and 1% to 99% of the hydrophobic moiety that comprises saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or
a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or
a combination thereof.

In some cases, the polymer can comprise the first polymer as disclosed herein.

In some cases, the polymer can consist of the first polymer, as disclosed herein. In some cases, the pharmaceutical composition can be free from the polymers selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

In some cases, in a range of from about 1% to 100% of the second terminal group can be free from primary amine. In some cases, the nanoaggregate disclosed herein, in a range of from 1% to 100% of the second terminal group can comprise hydroxyl group. All percentages are based on the total number of the second terminal groups.

In some cases, the polymer can comprise the second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

In some cases, the polymer can comprise the first polymer and the second polymer comprising one or more of subsequent polymers selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

In some cases, the bioactive agent can comprise any one of the bioactive agents listed above and hereafter. In some cases, the bioactive agent can comprise two or more of the bioactive agents listed above and hereafter. In some cases, the bioactive agent can comprise a natural or synthetic small molecule-based drug, inorganic-based drug, biological drug, natural or synthetic large molecule-based drug, a derivative thereof, or a combination thereof. In some cases, the bioactive agent can comprise taxane, paclitaxel, docetaxel, cabazitaxel, larotaxel, milataxel, ortataxel, tesetaxel, Topoisomerase 1 (Top 1) inhibitor, camptothecin derivative, irinotecan (CPT-11), SN-38, topotecan, Topoisomerase 2 (Top 2) inhibitor, doxorubicin, etoposide, ciprofloxaxin, mTOR inhibitor, at least one STING polypeptide or a part thereof, a nucleic acid encoding the STING polypeptide or a part thereof, a STING inhibitor, a STING activator, a STING agonist, a STING antagonist, a STING modulating molecule, an IDO inhibitor, an IDO1 inhibitor, or a combination thereof, wherein the mTOR inhibitor comprises everolimus, rapamycin, temsirolimus, zotarolimus, torin-1, torin-2, vistusertib, ridaforolimus, one or more dual PI3K-mTOR inhibitors, one or more ATP-competitive mTORC1/2 inhibitors, a derivative thereof, or a combination thereof. In some cases, the bioactive agent can comprise 7-ethyl-10-hydroxycamptothecin (SN-38).

The bioactive agent can comprise at least a compound having Formula (1)-Formula (29) (FIG. 12A-FIG. 12E), a pharmaceutically acceptable salt thereof, solvate thereof, prodrug thereof, isomer thereof, or a combination thereof.

In some cases, the bioactive agent can comprise a compound having Formula (1):

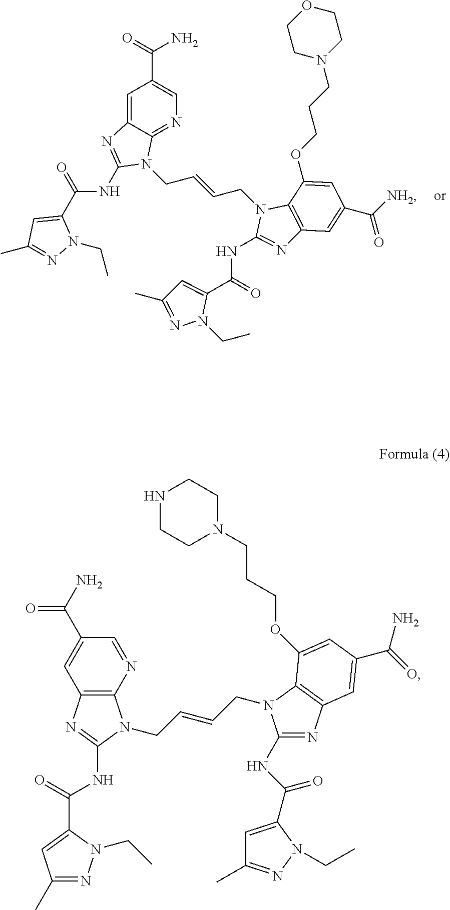 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

a pharmaceutically acceptable salt thereof, solvate thereof, prodrug thereof, isomer thereof, or a combination thereof.

In some cases, the disease can comprise one or more immune disorders, infectious diseases, cancers, and a combination thereof.

In some cases, the polymer can comprise a polyoxazoline (POX) that comprises a linear portion, a branched portion, or a combination thereof, and wherein the polyoxazoline (POX) comprises poly(2-oxazoline), poly(2-methyloxazoline), poly(2-ethyloxazoline), poly(2-propyloxazoline), poly(isopropyloxazoline), or a combination thereof.

Applicants unexpected discovered that when a polymer comprises certain amount of the first terminal group comprising H, an improved nanoaggregate formation can be achieved. Applicants further discovered that when a polymer comprises certain amounts of the first terminal group comprising H as disclosed herein, an improved manufacturing process, such as, shorter production cycling time, less mixing energy, and other benefits, can be achieved.

The instant disclosure now will be exemplified in the following non-limiting examples.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Materials and Measurements

### Polymers

Hydrocarbon (CH₃(CH₂)₁₇)-modified randomly branched PEOX polymer having monomer to initiator molar ratio=60:1 (herein “C₁₈PEOXABP60”) was prepared using initiator (CH₃(CH₂)₁₇)—Br as described previously in PCT Publication No.: WO2014/123791, herein incorporated by reference.

Another hydrocarbon modified randomly branched PEOX polymer having monomer to initiator molar ratio=60:1 was prepared as described above with an initiator (CH₃(CH₂)₅)—Br.

Another hydrocarbon modified randomly branched PEOX polymer having monomer to initiator molar ratio=60:1 was prepared as described above with an initiator (CH₃(CH₂)₁₁)—Br.

Another hydrocarbon modified randomly branched PEOX polymer having monomer to initiator molar ratio=60:1 was prepared as described above with an initiator methyl tosylate.

Hydrogen modified randomly branched PEOX polymer having monomer to initiator molar ratio=60:1 (herein “H-PEOXABP60”) was prepared as described above with an initiator p-Toluenesulfonic acid.

Another hydrogen modified randomly branched PEOX polymer having monomer to initiator molar ratio=60:1 was prepared as described above with an initiator trifluoroacetic acid.

The hydrogen modified randomly branched PEOX polymers having monomer to initiator molar ratio=60:1 are collectively referred to as “H-PEOXABP60”.

Table 1 shows some non-limiting examples of polymers with various first terminals modified with H (H-PEOXABP60) and C₁₈ hydrocarbon (C₁₈PEOXABP60). The presence of the H and C₁₈ hydrocarbon modified first terminal groups were measured with HPLC. Molar ratios of H to C₁₈ hydrocarbon and percent of H are shown.

TABLE 1
Ratios of H to C₁₈hydrocarbon and percent of H of Mixed Polymer
Examples H-PEOXABP60 and C18PEOXABP60 (Molar ratio).

| | H to C18 Ratio | % of H |
|---|---|---|
| Polymers | (H/C₁₈ Molar Ratio) | (Molar Percent) |
| Polymer 1 | 0.10 | 9% |
| Polymer 2 | 0.33 | 25% |
| Polymer 3 | 0.52 | 34% |
| Polymer 4 | 0.65 | 39% |
| Polymer 5 | 0.76 | 43% |
| Polymer 6 | 0.99 | 50% |
| Polymer 7 | 1.02 | 50% |
| Polymer 8 | 1.49 | 60% |

Polymers comprising a mixture of hydrocarbon (CH₃(CH₂)₁₇)-modified first terminals and H modified first terminals with an initiator molar ratio=60:1, herein “H/C₁₈PEOXABP60”, were used for producing the nanoaggregates.

A polymer, H/C₁₈PEOXABP60, having an H/C₁₈ of about 0.3 was terminated in water with the hydroxyl group as the second terminal group (herein referred to as “Polymer A1”). Aqueous solutions of the Polymer had pH values in a range of from 3.0 to 6.9. If needed, an aqueous solution of the Polymer can be adjusted to have pH values in a range of from 5.6 to 7.5 or 3.0 to 10 using HCl or NaOH.

A polymer, H/C₁₈PEOXABP60, having an H/C₁₈ of about 0.4 was terminated in water with the hydroxyl group as the second terminal group (herein referred to as “Polymer A2”). An aqueous solution of the Polymer can be adjusted to have pH values in a range of from 3.0 to 6.9. Another aqueous solution of the Polymer can be adjusted to have pH values in a range of from 7.0 to 10 or 5.6 to 7.5 using HCl or NaOH.

A polymer, H/C₁₈PEOXABP60, having an H/C₁₈ of about 0.7 was terminated in water with the hydroxyl group as the second terminal group (herein referred to as “Polymer A3”). Aqueous solutions of the Polymer had pH values in a range of from 3.0 to 6.9. If needed, an aqueous solution of the Polymer can be adjusted to have pH values in a range of from 7.0 to 10 or 5.6 to 7.5 using HCl or NaOH.

All H/C₁₈PEOXABP60 polymers having a hydroxyl group as the second terminal group can be referred to as H/C₁₈PEOXABP60-OH.

A polymer, H/C₁₈PEOXABP60, having an H/C₁₈ of about 0.7 was terminated with EDA with a molar ratio of polyoxazoline reactive chain end to EDA at a ratio of about 1:10, producing a polymer having the second terminal group comprising a group modified with a primary amine (herein referred to as “Polymer B1”). Aqueous solutions of the Polymer had pH values in a range of from 7.0 to 10. Another aqueous solution of the Polymer can be adjusted to have pH values in a range of from 8.9 to 9.7 using HCl or NaOH. Another aqueous solution of the Polymer can be adjusted to have pH values in a range of from 3.0 to 6.9.

A polymer, H/C₁₈PEOXABP60, having an H/C₁₈ of about 0.4 was terminated with EDA with a molar ratio of polyoxazoline reactive chain end to EDA at a ratio of about 1:10, producing a polymer having the second terminal group comprising a group modified with a primary amine (herein referred to as “Polymer B2”). An aqueous solution of the Polymer can be adjusted to have pH values in a range of from 7.0 to 10. Another aqueous solution of the Polymer can be adjusted to have pH values in a range of from 3.0 to 10 or 5.6 to 7.5 using HCl or NaOH.

A polymer, H/C₁₈PEOXABP60, having an H/C₁₈ of about 0.3 was terminated with EDA with a molar ratio of polyoxazoline reactive chain end to EDA at a ratio of about 1:10, producing a polymer having the second terminal group comprising a group modified with a primary amine (herein referred to as “Polymer B3”). An aqueous solution of the Polymer can be adjusted to have pH values in a range of from 7.0 to 10. Another aqueous solution of the Polymer can be adjusted to have pH values in a range of from 3.0 to 10 or 5.6 to 7.5 using HCl or NaOH.

All H/C₁₈PEOXABP60 polymers having an —NH₂ group as the second terminal group can be referred to as H/C₁₈PEOXABP60-NH₂.

### Nanoparticle Measurement

The size of various polymers, polymer-only nanoaggregates, as well as drug-induced polymer-drug nanoaggregates was measured by a dynamic light scattering (DLS) method using a Malvern Zetasizer Nano-ZS Zen3600 particle size analyzer (Malvern Panalytical Inc., Westborough, MA 01581, USA).

### Examples 1-2

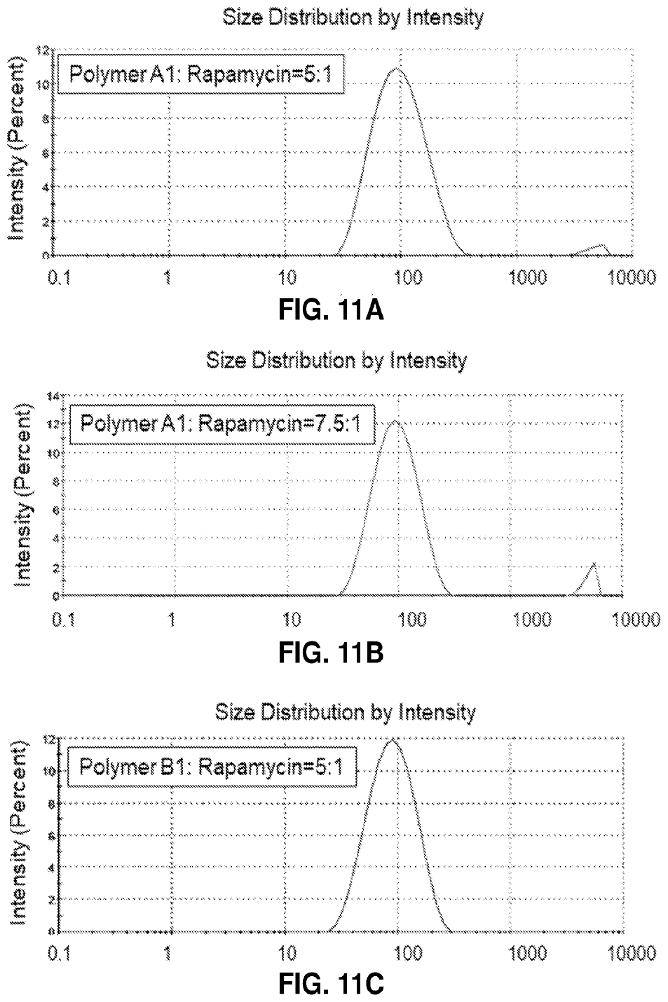

Nanoparticle with H/C₁₈PEOXABP60-A (Polymer A1) Polymer:Rapamycin H/C₁₈PEOXABP60-A (Polymer A1) (750 mg) and Rapamycin (150 mg) were dissolved in 5 mL methanol for about 20 minutes. Methanol was then removed using a rotary evaporator to produce dried nanoaggregates. The dried nanoaggregates powder was reconstituted into water to produce an aqueous nanoaggregate solution containing 3.5 mg/mL or 5 mg/mL rapamycin (weight ratio of polymer:rapamycin=5:1). An example of light scattering (LS) measurement data is shown in FIG. 11A. The nanoaggregates were of a size in a range of from 100 to about 106 nm.

H/C₁₈PEOXABP60-A (Polymer A1) (750 mg) and Rapamycin (100 mg) were dissolved in 5 mL methanol and processed according to the process described above to produce aqueous nanoaggregate solution containing 3.5 mg/mL or 5 mg/mL rapamycin having a weight ratio of polymer:rapamycin=7.5:1. An example of LS measurement data is shown in FIG. 11B. The nanoaggregates were of a size in a range of from 100 to about 102 nm.

The aqueous nanoaggregate solutions were each passed through a 0.8 μm filter and then a 0.22 μm filter. The filtrate, i.e., sterilized nanoaggregate, was aliquoted and lyophilized over 24 hours depending on the amount used to produce lyophilized nanoaggregates. The vial was stoppered and the ready-to-use white powder was stored at room temperature.

### Example 3

Nanoparticle with H/C₁₈PEOXABP60-NH₂ (Polymer B1) Polymer:Rapamycin

H/C₁₈PEOXABP60-NH₂ (Polymer B1) (750 mg) and Rapamycin (150 mg) were dissolved in 5 mL methanol and processed according to the process described above to produce an aqueous nanoaggregate solution containing 5 mg/mL rapamycin having a weight ratio of polymer:rapamycin=5:1. An example of LS measurement data is shown in FIG. 11C. The nanoaggregates in the finished product, i.e., after lyophilization, were of a size in a range of from 91 to about 112 nm.

The aqueous nanoaggregate solution was passed through a 0.8 μm filter and then a 0.22 μm filter. The filtrate, i.e., sterilized nanoaggregate, was aliquoted and lyophilized over 24 hours depending on the amount used to produce lyophilized nanoaggregates. The vial was stoppered and the ready-to-use white powder was stored at room temperature.

### Example 4

### Rapamycin Stability Assay

Stability of the nanoaggregates prepared in Examples 1-3 was measured by HPLC at RT for a time period of 0-147 hours for degradation products and impurities of rapamycin.

Nanoaggregates prepared in Examples 1-3 had a good stability over the time period measured.

### Example 5

Nanoparticle with H/C₁₈PEOXABP60-A (Polymer A1) Polymer:Formula (1)

Polymer A1 (100 mg) having an H/C₁₈ of about 0.3 prepared above is mixed with the compound of Formula (1) (2 mg) in 1 mL water and processed according to the process described above to produce aqueous nanoaggregate solution containing 2 mg/mL Formula (1) having a weight ratio of polymer: Formula (1)=50:1.

### Example 6

Nanoparticle with H/C₁₈PEOXABP60-A (Polymer A3) Polymer:Formula (1)

Polymer A3 (500 mg) having H/C₁₈ of about 0.7 prepared above is mixed with the compound of Formula (1) (10 mg) in 5 mL water and processed according to the process described above to produce an aqueous nanoaggregate solution containing 2 mg/mL Formula (1) having a weight ratio of polymer: Formula (1)=50:1.

### Example 7

Nanoparticle with H/C₁₈PEOXABP60-NH₂ (Polymer B3) Polymer:Paclitaxel Ratio 3:1 to 10:1

As a general procedure, paclitaxel was dissolved in methanol to a concentration of up to 40 mg/mL. Polymer B3 was separately dissolved to a concentration of up to 100 mg/mL in methanol. The two solutions were then mixed at various volumes to result in final polymer to paclitaxel weight ratios in the mixtures ranging from 3:1 to 10:1. The mixtures subsequently were lyophilized.

The size of the aggregates as measured by light scattering ranged from about 70 nm to 90 nm in diameter before lyophilization and 120-140 nm after lyophilization.

Alternatively, both paclitaxel and the Polymer B3 can be dissolved in a common solvent, such as, acetone, methanol, or ethanol and then dropwise added to water while being stirred or sonicated, followed by sterile filtration with a 0.22 μm filter. The final product then can be generated by lyophilization and the size of the aggregates measured by light scattering.

Other taxane-induced aggregates or nanoparticles using various hydrophobically surface-modified branched polymers, such as, C4, C6, C12 or C22 hydrocarbon-modified randomly branched PEOX, PEI and PPI polymers: C4, C6, C12, C18 and C22 hydrocarbon-modified PAMAM, PEI and PPI dendrimers and dendrigrafts; and C4, C6, C12, C18 and C22 hydrocarbon-modified branched PLL/polymers can be prepared in a similar manner.

### Example 8

Nanoparticle with H/C₁₈PEOXABP60-NH₂ (Polymer B3) Polymer:Paclitaxel Ratio 7:1

H/C₁₈PEOXABP60-NH₂ (Polymer B3) (700 mg) was dissolved in 9.33 mL of methanol to yield a 75 mg/mL solution. A 15 mg/mL solution of paclitaxel was also prepared by dissolving 100 mg in 6.67 mL of methanol. The two solutions were mixed for 20 minutes resulting in a solution containing 6.25 mg paclitaxel and 43.75 mg polymer per mL, providing a solution with a 7:1 polymer:drug ratio. The mixture was placed on a rotary evaporator and the methanol removed to dryness. The resultant solid was redissolved with stirring in 33.3 ml of water to a final paclitaxel concentration of 3 mg/mL. The solution preparation was passed through a 0.8 μm filter and then a 0.22 μm filter. The filtrate was lyophilized. The vial was stoppered and the ready-to-use white powder was stored at room temperature.

### Examples 9-11

Nanoparticle with H/C₁₈PEOXABP60-A (Polymer A2) Polymer:(SN-38/irinotecan)

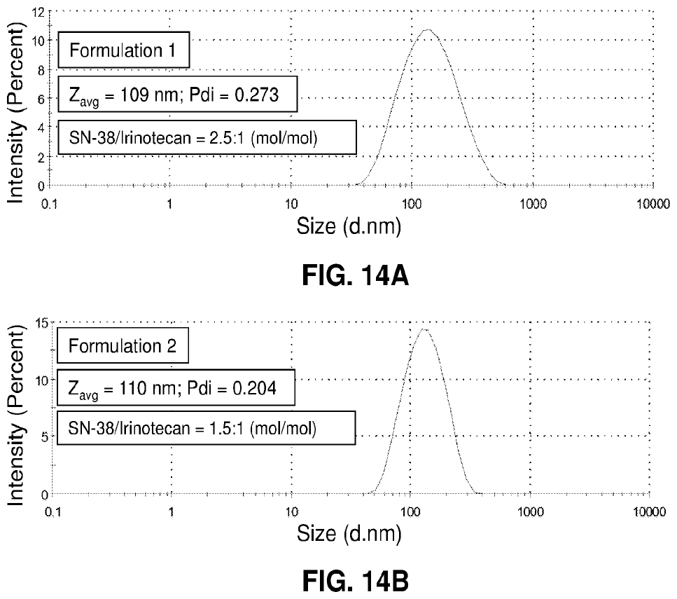

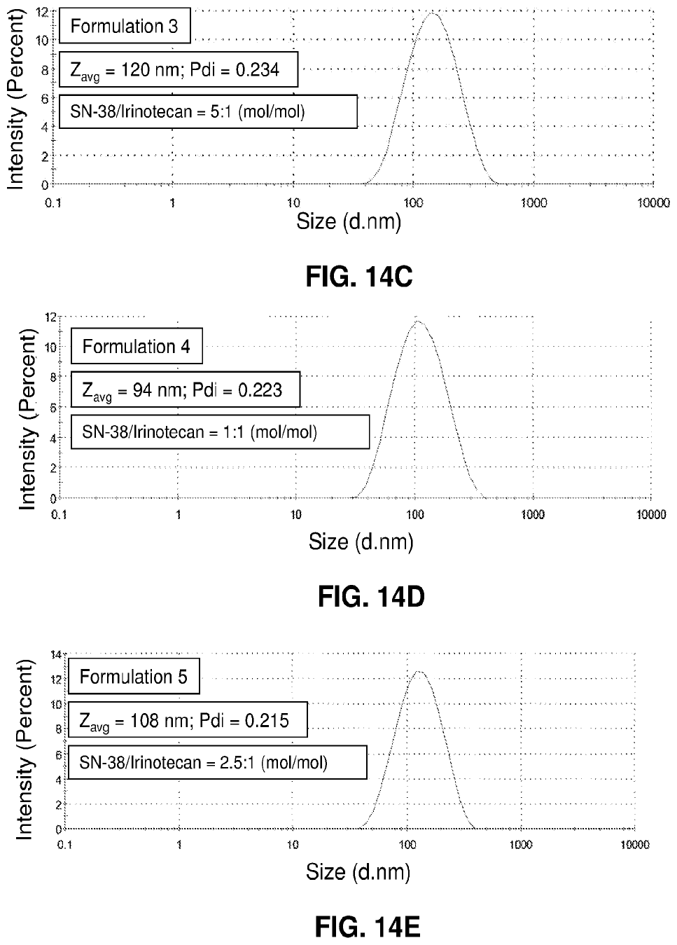

H/C₁₈PEOXABP60-A (Polymer A2) (981 mg) and SN-38 (149 mg) and irinotecan (97 mg) were dissolved in 70 mL of solvent (THF:methanol, 6:1, v/v) for about 20 minutes at 50° C. under stirring. Organic solvent was then removed using a rotary evaporator to produce dried nanoaggregates. The dried nanoaggregate powder was reconstituted into 5% glucose to produce an aqueous nanoaggregate solution containing 2 mg/mL of total API (weight ratio of polymer:(SN-38/irinotecan)=4:1 with molar ratio of SN-38/irinotecan=2.5/1). An example of LS measurement data is shown in FIG. 14A (Formulation 1). The nanoaggregates were of a size of about 110 nm.

H/C₁₈PEOXABP60-A (Polymer A2) (60 mg) and SN-38 (11.4 mg) and irinotecan (12.2 mg) were dissolved in 7 mL of solvent (THF:methanol, 6:1, v/v) for about 20 minutes at 50° C. under stirring. Organic solvent was then removed using a rotary evaporator to produce dried nanoaggregates. The dried nanoaggregate powder was reconstituted into 5% glucose to produce an aqueous nanoaggregate solution containing 2 mg/mL of total API (weight ratio of polymer:(SN-38/irinotecan)=2.6:1 with a molar ratio of SN-38/irinotecan=1.5/1). An example of LS measurement data is shown in FIG. 14B (Formulation 2). The nanoaggregates were of a size of about 110 nm.

H/C₁₈PEOXABP60-A (Polymer A2) (60 mg) and SN-38 (11.4 mg) and irinotecan (3.6 mg) were dissolved in 7 mL of solvent (THF:methanol, 6:1, v/v) for about 20 minutes at 50° C. under stirring. Organic solvent was then removed using a rotary evaporator to produce dried nanoaggregates. The dried nanoaggregate powder was reconstituted into 5% glucose to produce an aqueous nanoaggregate solution containing 2 mg/mL of total API (weight ratio of polymer:(SN-38/irinotecan)=4:1 with a molar ratio of SN-38/irinotecan=5/1). An example of LS measurement data is shown in FIG. 14C (Formulation 3). The nanoaggregates were of a size of about 120 nm.

H/C₁₈PEOXABP60-A (Polymer A1) (5.25 g) and SN-38 (1.16 g) and irinotecan (1.84 g) were dissolved in 600 mL of solvent (THF:methanol, 6:1, v/v) for about 30 minutes at 50° C. under stirring. Organic solvent was then removed using a rotary evaporator to produce dried nanoaggregates. The dried nanoaggregate powder was reconstituted into 5% glucose to produce an aqueous nanoaggregate solution containing 2 mg/mL of total API (weight ratio of polymer:(SN-38/irinotecan)=1.75:1 with a molar ratio of SN-38/irinotecan=1/1). An example of LS measurement data is shown in FIG. 14D (Formulation 4). The nanoaggregates were of a size of about 94 nm.

Nanoparticle with H/C₁₈PEOXABP60-B (Polymer B1) Polymer:(SN-38/irinotecan)

H/C₁₈PEOXABP60-B (Polymer B1) (5.25 g) and SN-38 (1.83 g) and irinotecan (1.17 g) were dissolved in 600 mL of solvent (THF:methanol, 6:1, v/v) for about 30 minutes at 50° C. under stirring. Organic solvent was then removed using a rotary evaporator to produce dried nanoaggregates. The dried nanoaggregate powder was reconstituted into 5% glucose to produce an aqueous nanoaggregate solution containing 2 mg/mL of total API (weight ratio of polymer:(SN-38/irinotecan)=1.75:1 with a molar ratio of SN-38/irinotecan=2.5/1). An example of LS measurement data is shown in FIG. 14E (Formulation 3). The nanoaggregates were of a size of about 108 nm.

The aqueous nanoaggregate solution was passed through a 0.8 μm filter and then a 0.2 μm filter. The filtrate, i.e., sterilized nanoaggregate, was aliquoted and lyophilized over 24-100 hours depending on the amount used to produce lyophilized nanoaggregates. The vial was stoppered and the ready-to-use white powder was stored at room temperature.

### Example 12

Maximum Tolerated Dose (MTD) Study in mice

Severe combined immunodeficient (SCID) Mice were injected with Irinotecan/CPT-11, Nano SN-38 produced in Examples 10-12 at various amounts until an MTD was reached.

### Example 13

### Cell Based Cytotoxicity Assay

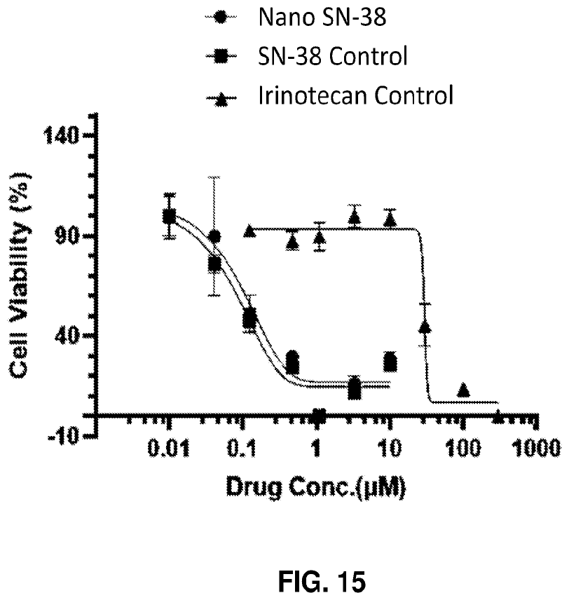

SN-38 was tested on the HCT-116 Cell line for cytotoxicity using a standard in vitro cytotoxicity assay. Cell viability was assessed with the Promega Cell Titer 96 Aqueous One kit. Overall, Nano-formulated SN-38 was about 250 times more toxic to the cells than was irinotecan, exhibiting a half maximal effective concentration (EC₅₀) toward the HCT-116 cells at about 0.13 μM while irinotecan having an EC₅₀ at about 32.6 μM (FIG. 15). Nano SN-38: produced in Examples above with a polymer:SN-38=4:1; SN-38/Irinotecan mixture: physical mixture of SN-38 and irinotecan at a molar ratio of 4:1, dissolved in DMSO; SN-38 Control: SN-38 dissolved in DMSO (dimethyl sulfoxide); and Irinotecan Control: irinotecan dissolved in water were tested on cells. Representative measurement data are shown in FIG. 15 and Table 2.

TABLE 2
Cytotoxicity Data.

| Formulation | Cell line | EC₅₀ (μM) |
|---|---|---|
| Nano SN-38 (4:1) | HCT-116 | 0.13 |
| SN-38/Irinotecan mixture | HCT-116 | 0.11 |
| Irinotecan Control | HCT-116 | 32.6 |
| SN-38 Control | HCT-116 | 0.10 |

### Example 14

Nanoparticle with H/C₁₈PEOXABP60-A (Polymer A1) Polymer:Formula (1)

Polymer A1 (200 mg) having an H/C₁₈ of about 0.4 prepared as above was dissolved in water to make a 100 mg/g solution. To the polymer solution was added the compound of Formula (1) (8 mg) and the mixture was processed according to the process described above to produce an aqueous nanoaggregate solution containing 2 mg/mL Formula (1) having a weight ratio of polymer:Formula (1)=25:1. The solution was filtered through a 0.8 μm filter and then a 0.22 μm filter, and then lyophilized over 20-100 h to provide a lyophilized dry power. The vial was stoppered and the ready-to-use white powder was stored at room temperature.

Polymer A1 (400 mg) having an H/C₁₈ of about 0.4 prepared as above was dissolved in water to make a 100 mg/g solution. To the polymer solution was added the compound of Formula (1) (8 mg) and the mixture was processed according to the process described above to produce an aqueous nanoaggregate solution containing 2 mg/mL Formula (1) having a weight ratio of polymer:Formula (1)=50:1. The solution was filtered through a 0.8 μm filter and then a 0.22 μm filter, and then lyophilized over 20-100 h to provide a lyophilized dry power. The vial was stoppered and the ready-to-use white powder was stored at room temperature.

Nanoparticle with H/C₁₈PEOXABP60-A (Polymer A2) Polymer:Formula (1)

Polymer A2 (400 mg) having an H/C₁₈ of about 0.4 prepared as above was dissolved in water to make a 100 mg/g solution. To the polymer solution was added the compound of Formula (1) (8 mg) and the mixture was processed according to the process described above to produce an aqueous nanoaggregate solution containing 2 mg/mL Formula (1) having a weight ratio of polymer:Formula (1)=50:1. The solution was filtered through a 0.8 μm filter and then a 0.22 μm filter, and then lyophilized over 20-100 h to provide a lyophilized dry power. The vial was stoppered and the ready-to-use white powder was stored at room temperature.

Polymer A3 (400 mg) having an H/C₁₈ of about 0.7 prepared as above was dissolved in water to make a 100 mg/g solution. To the polymer solution was added the compound of Formula (1) (8 mg) and the mixture was processed according to the process described above to produce an aqueous nanoaggregate solution containing 2 mg/mL Formula (1) having a weight ratio of polymer:Formula (1)=50:1. The solution was filtered through a 0.8 μm filter and then a 0.22 μm filter, and then lyophilized over 20-100 h to provide a lyophilized dry power. The vial was stoppered and the ready-to-use white powder was stored at room temperature.

### Example 15

Polymer B3 (400 mg) having an H/C₁₈ of about 0.3 prepared as above was dissolved in water to make a 100 mg/g solution. To the polymer solution was added the compound of Formula (1) (8 mg) and the mixture was processed according to the process described above to produce an aqueous nanoaggregate solution containing 2 mg/mL Formula (1) having a weight ratio of polymer:Formula (1)=50:1. The solution was filtered through a 00.8 μm filter and then a 0.22 μm filter, and then lyophilized over 20-100 h to provide a lyophilized dry power. The vial was stoppered and the ready-to-use white powder was stored at room temperature.

Polymer B3 (200 mg) having an H/C₁₈ of about 0.3 prepared as above was dissolved in water to make a 100 mg/g solution. To the polymer solution was added the compound of Formula (1) (8 mg) and the mixture was processed according to the process described above to produce an aqueous nanoaggregate solution containing 2 mg/mL Formula (1) having a weight ratio of polymer:Formula (1)=25:1. The solution was filtered through a 0.8 μm filter and then a 0.22 μm filter, and then lyophilized over 20-100 h to provide a lyophilized dry power. The vial was stoppered and the ready-to-use white powder was stored at room temperature.

1.-79. (canceled)

80. A nanoaggregate comprising a polymer and at least one bioactive agent that is water insoluble or poorly water soluble;
wherein said nanoaggregate is soluble in an aqueous solution to produce at least 1 mg/mL of said bioactive agent in said aqueous solution;
wherein said polymer is water soluble; and
wherein said polymer comprises:
a first polymer comprising at least one first terminal group modified with H or a hydrophobic moiety and a second terminal group modified with a hydrophilic moiety, wherein said first terminal group comprises in a range of from 1% to 99% of H and 1% to 99% of said hydrophobic moiety that comprises saturated or unsaturated aliphatic hydrocarbon having 1 to about 22 carbons, an aromatic hydrocarbon, or a combination thereof, and said second terminal group comprises a group modified by an amine, amide, imine, imide, carboxyl, hydroxyl, ester, ether, acetate, phosphate, ketone, aldehyde, sulfonate, or a combination thereof; or
a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); an amphiphilic nonionic triblock copolymer comprising a central polypropylene (PPO) block flanked on either side by polyethylene (PEO) blocks; poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or
a combination thereof.

81. The nanoaggregate of claim 80, wherein said polymer comprises said first polymer.

82. The nanoaggregate of claim 80, wherein said polymer comprises said second polymer.

83. The nanoaggregate of claim 80, wherein said polymer comprises a polyoxazoline (POX).

84. The nanoaggregate of claim 80, wherein said polymer comprises a linear POX, a branched POX or a combination thereof.

85. The nanoaggregate of claim 80, wherein said polymer comprises poly(2-methyloxazoline), poly(2-ethyloxazoline), poly(2-propyloxazoline), poly(isopropyloxazoline), or a combination thereof.

86. The nanoaggregate of claim 80, wherein said polymer comprises poly(2-ethyloxazoline).

87. The nanoaggregate of claim 80, wherein said polymer comprises polyoxazoline, wherein said polyoxazoline comprises a molar ratio of monomer to initiator in a range of from 50:1 to 80:1.

88. The nanoaggregate of claim 80, wherein from 1% to 100% of said second terminal group is free from primary amine.

89. The nanoaggregate of claim 80, wherein from 1% to 100% of said second terminal group comprises a hydroxyl group.

90. The nanoaggregate of claim 80, wherein said nanoaggregate is of a size less than 120 nm before lyophilization.

91. The nanoaggregate of claim 80, wherein said nanoaggregate comprises a weight ratio of said polymer to said bioactive agent in a range of from about 2:1 to about 200:1.

92. The nanoaggregate of claim 80, wherein said nanoaggregate further comprises a targeting moiety, wherein said targeting moiety comprises an antibody, an antigen-binding portion thereof, an antigen, a cell receptor, a cell receptor ligand, a ligand for a cellular protein, a ligand for a membrane protein, a small molecule ligand, a lectin ligand, or a combination thereof.

93. The nanoaggregate of claim 80, wherein said nanoaggregate is free from human serum albumin, organic solvent, detergent, or oil.

94. The nanoaggregate of claim 80, wherein said bioactive agent comprises a natural or synthetic small molecule-based drug, inorganic-based drug, biological drug, natural or synthetic large molecule-based drug, a drug derivative, or a combination thereof.

95. The nanoaggregate of claim 80, wherein said bioactive agent comprises a taxane, paclitaxel, docetaxel, cabazitaxel, larotaxel, milataxel, ortataxel, tesetaxel, a topoisomerase 1 (Top 1) inhibitor, a camptothecin derivative, irinotecan (CPT-11), SN-38, topotecan, a topoisomerase 2 (Top 2) inhibitor, doxorubicin, an etoposide, ciprofloxaxin, an mTOR inhibitor, at least one stimulator of an interferon gene (STING) polypeptide or a part thereof, a nucleic acid encoding said STING polypeptide or a part thereof, a STING inhibitor, a STING activator, a STING agonist, a STING antagonist, a STING modulating molecule, an indoleamine 2,3-dioxygenase (IDO) inhibitor, an IDO1 inhibitor, or a combination thereof, wherein said mTOR inhibitor comprises everolimus, rapamycin, temsirolimus, zotarolimus, torin-1, torin-2, vistusertib, ridaforolimus, one or more dual PI3K-mTOR inhibitors, one or more ATP-competitive mTORC1/2 inhibitors, a derivative thereof, or a combination thereof.

96. The nanoaggregate of claim 80, wherein said bioactive agent comprises at least a compound having one of Formula (1)-Formula (29).

97. The nanoaggregate of claim 80, wherein said bioactive agent comprises a compound having Formula (1)

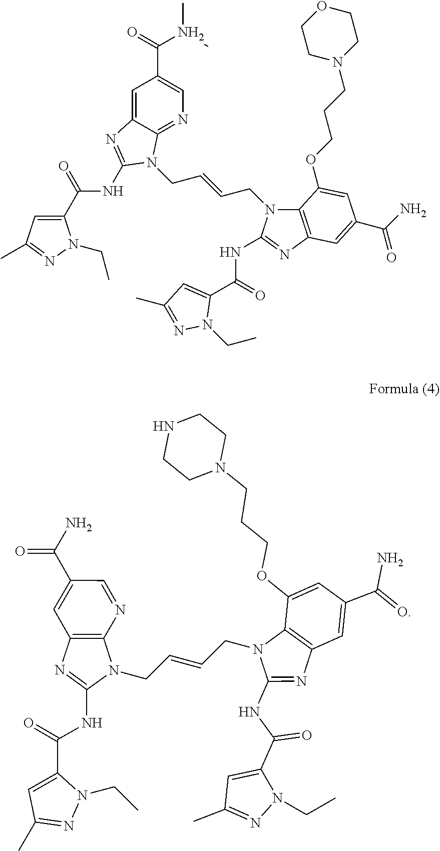 [SMILES: CCn1nc(C)cc1C(=O)Nc1nc2cc(C(=O)N(C)C)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

98. The nanoaggregate of claim 80, which is an adjuvant.

99. The nanoaggregate of claim 80, wherein said nanoaggregate has a weight ratio of said polymer to said bioactive agent in a range of from about 5:1 to about 8:1.

100. A pharmaceutical composition comprising the nanoaggregate of claim 80 and optionally a pharmaceutical suitable carrier;
wherein said pharmaceutical composition is soluble in said aqueous solution to produce at least 1 mg/mL of said bioactive agent in said aqueous solution.

101. The pharmaceutical composition of claim 100 which is free from human serum albumin, organic solvent, detergent, or oil.

102. The pharmaceutical composition of claim 100, wherein said bioactive agent comprises a natural or synthetic small molecule-based drug, inorganic-based drug, biological drug, natural or synthetic large molecule-based drug, a derivative thereof, or a combination thereof.

103. The pharmaceutical composition of claim 100, wherein said bioactive agent comprises taxane, paclitaxel, docetaxel, cabazitaxel, larotaxel, milataxel, ortataxel, tesetaxel, Topoisomerase 1 (Top 1) inhibitor, camptothecin derivative, irinotecan (CPT-11), SN-38, topotecan, Topoisomerase 2 (Top 2) inhibitor, doxorubicin, etoposide, ciprofloxaxin, mTOR inhibitor, at least one STING polypeptide or a part thereof, a nucleic acid encoding said STING polypeptide or a part thereof, a STING inhibitor, a STING activator, a STING agonist, a STING antagonist, a STING modulating molecule, an IDO inhibitor, an IDO1 inhibitor, or a combination thereof, wherein said mTOR inhibitor comprises everolimus, rapamycin, temsirolimus, zotarolimus, torin-1, torin-2, vistusertib, ridaforolimus, one or more dual PI3K-mTOR inhibitors, one or more ATP-competitive mTORC1/2 inhibitors, a derivative thereof, or a combination thereof.

104. The pharmaceutical composition of claim 100, further comprising an immune agent for stimulating an immune response.

105. The pharmaceutical composition of claim 104, wherein said immune agent comprises an inactive microbe selected from bacterium, virus, fungus, protozoan, worm, parasite, prion, a part thereof, or a combination thereof; a toxin; a nucleic acid encoding a toxin; a protein; a nucleic acid encoding a protein; an oligo nucleic acid; a DNA; an RNA; an mRNA; an siRNA; an sgRNA; fragments thereof; or a combination thereof.

106. A method for treating or preventing a disease of a subject in need thereof, said method comprising administering to said subject an effective dose of a pharmaceutical composition of claim 100.

107. The method of claim 106, wherein said bioactive agent comprises a natural or synthetic small molecule-based drug, inorganic-based drug, biological drug, natural or synthetic large molecule-based drug, a derivative thereof, or a combination thereof.

108. The method of claim 106, wherein said bioactive agent comprises taxane, paclitaxel, docetaxel, cabazitaxel, larotaxel, milataxel, ortataxel, tesetaxel, Topoisomerase 1 (Top 1) inhibitor, camptothecin derivative, irinotecan (CPT-11), SN-38, topotecan, Topoisomerase 2 (Top 2) inhibitor, doxorubicin, etoposide, ciprofloxaxin, mTOR inhibitor, at least one STING polypeptide or a part thereof, a nucleic acid encoding said STING polypeptide or a part thereof, a STING inhibitor, a STING activator, a STING agonist, a STING antagonist, a STING modulating molecule, an IDO inhibitor, an IDO1 inhibitor, or a combination thereof, wherein said mTOR inhibitor comprises everolimus, rapamycin, temsirolimus, zotarolimus, torin-1, torin-2, vistusertib, ridaforolimus, one or more dual PI3K-mTOR inhibitors, one or more ATP-competitive mTORC1/2 inhibitors, a derivative thereof, or a combination thereof.

109. The method of claim 106, wherein said pharmaceutical composition is administered to said subject via intravenous (IV) injection, subcutaneous (SC) injection, intramuscular (IM) injection, intradermal (ID) injection, or a combination thereof.

110. The method of claim 106 further comprising the step of administering said subject with one or more subsequent bioactive agents selected from a protein, a peptide, an antibody, a fragment of an antibody, a chemical compound, a small molecule drug, one or more chemotherapy drugs, a vaccine, and a combination thereof, wherein each of said one or more subsequent bioactive agents is administered to the subject, prior to, at the same time as, or after administering said pharmaceutical composition.

111. The method of claim 106, wherein said one or more subsequent bioactive agents are selected from a gemcitabine, a taxane, paclitaxel, docetaxel, cabazitaxel, larotaxel, milataxel, ortataxel, tesetaxel, Topoisomerase 1 (Top 1) inhibitor, camptothecin derivative, irinotecan (CPT-11), SN-38, topotecan, Topoisomerase 2 (Top 2) inhibitor, doxorubicin, etoposide, ciprofloxaxin, a platinum-based antineoplastic agent, anti-PD1 antibodies, anti-PD-L1 antibodies, anti-CTLA-4 (cytotoxic T lymphocyte-associated antigen) antibodies, anti-LAG3 (lymphocyte activation gene-3) antibodies, anti-TIM-3 (T cell immunoglobulin and mucin domain-3) antibodies, anti-CD19 antibodies, anti-CD20 antibodies, cytokines, interleukins, interferon α2a, interferon α, granulocyte colony stimulating factor (G-CSF), Neupogen or Filgrastim, T-cell receptor (TCR), chimeric antigen receptor or chimeric antigen T-cell receptor (CAR-T), a vaccine, and a combination thereof.
